(19) United States
(12) Patent Application Publication
(10) Pub. No.: US 2004/0048910 A1
(43) Pub. Date: Mar. 11, 2004

(54) Method of treating osteoarthritis

(76) Inventors: Susan Elizabeth Bove, Ann Arbor, MI (US); Joseph Anthony Cornicelli, Ann Arbor, MI (US); Kenneth Kilgore, Ann Arbor, MI (US); Mark Charles Kowala, Ann Arbor, MI (US); David Herbert Neideffer, Ann Arbor, MI (US); Drago Robert Sliscovic, Saline, MI (US)

Correspondence Address: WARNER-LAMBERT COMPANY, 2800 PLYMOUTH RD, ANN ARBOR, MI, 48105, US

(21) Appl. No.: 10/639,719

(22) Filed: Aug. 12, 2003

Related U.S. Application Data
(60) Provisional application No. 60/405,250, filed on Aug. 22, 2002.
(60) Provisional application No. 60/475,443, filed on Jun. 3, 2003.
(60) Provisional application No. 60/477,092, filed on Jun. 9, 2003.
(60) Provisional application No. 60/484,808, filed on Jul. 3, 2003.

(51) Int. Cl. A61K 31/41; A61K 31/19; A61K 31/11

(52) U.S. Cl. 514/381; 514/557; 514/693

(57) ABSTRACT

This invention relates to combinations, compositions, and methods using or having a substituted dialkyl ether, substituted aryl-alkyl ether, substituted dialkyl thioether, substituted dialkyl ketone, or substituted-alkyl compound, or a pharmaceutically acceptable salt thereof, as an active component for preventing or treating osteoarthritis, preventing or inhibiting cartilage damage, preventing or treating rheumatoid arthritis, improving joint function, alleviating pain, and the like in a patient in need thereof.

### CROSS-REFERENCE TO RELATED APPLICATIONS

This application claims benefit of priority from U.S. Provisional patent application Ser. No. 60/405,250 filed Aug. 22, 2002; Ser. No. 60/475,443 filed Jun. 3, 2003; Ser. No. 60/477,092 filed Jun. 9, 2003; and Ser. No. 60/484,808 filed Jul. 3, 2003.

This invention relates to methods, compositions, and combinations using or having a substituted dialkyl ether, substituted aryl-alkyl ether, substituted dialkyl thioether, substituted dialkyl ketone, or substituted-alkyl compound, or a pharmaceutically acceptable salt thereof, as an active component for preventing or treating osteoarthritis (“OA”), preventing or inhibiting cartilage damage, preventing or treating rheumatoid arthritis (“RA”), improving joint function, alleviating pain, including joint pain, and the like, in a patient in need thereof.

### BACKGROUND OF THE INVENTION

More than 100 million people worldwide are afflicted with some form of arthritis, a disabling, even crippling, disease or disorder of the joints or spine (ankylosing spondylitis). Depending on the type of the disease or disorder, a patient afflicted with an arthritis may or may not also be afflicted with additional conditions such as psoriasis (psoriatic arthritis), autoimmune conditions (e.g., systemic lupus erythematosus), gout, muscle disorders (e.g., fibromyalgia), a joint infection (infectious arthritis), scleroderma, or one or more of the following conditions: urethritis, prostatitis, cervicitis, cystitis, eye problems, or skin problems (Reiter's syndrome).

For example in the United States alone, more than 42 million people currently have some form of arthritis, including 300,000 children (e.g., juvenile rheumatoid arthritis), and the total number is expected to grow to 60 million people by the year 2020. Among the various forms of arthritis in the U.S., OA is the most prevalent, afflicting 21 million people. An OA patient suffering from cartilage damage primarily experiences pain and joint stiffness leading to joint deformities and diminishment or loss of joint function.

In patients suffering from RA, the clinical outcome relates to progressive destruction of cartilage and bone. In joints affected with RA, the chronically inflamed and hyperplastic synovial tissue invades adjacent cartilage and bone, leading to a loss of joint function and disability.

Aspirin and conventional nonsteroidal anti-inflammatory drugs (NSAIDs) such as ibuprofen, diclofenac, and naproxen, are the typical agents used to treat RA-related inflammation or RA- or OA-related pain. NSAIDs inhibit prostaglandin release by blocking cyclooxygenase-1 (COX-1) and/or cyclooxygenase-2 (COX-2) mediated conversion of cell membrane lipids from arachidonic acid. However, the therapeutic use of conventional NSAIDs is limited due to drug- and mechanism-associated side effects, including life threatening gastric ulceration and renal toxicity. Further, these drugs only treat secondary symptoms associated with cartilage damage, rheumatoid arthritis, or osteoarthritis such as pain. They do not prevent or treat the underlying pathophysiological condition, which is damage to the cartilage or bone. The need for new and improved therapies for these diseases thus continues.

We have now discovered that compounds which are substituted dialkyl ethers, substituted aryl-alkyl ethers, substituted dialkyl thioethers, substituted dialkyl ketones, or substituted-alkyl compounds, or a pharmaceutically acceptable salt thereof, including a compound named 6-(5-carboxy-5-methyl-hexyloxy)-2,2-dimethyl-hexanoic acid, calcium salt, are useful for preventing and inhibiting cartilage damage, preventing and treating arthritis, rheumatoid arthritis, improving joint function, alleviating pain, including joint pain, preventing and treating osteoarthritis, and the like. All that is required to practice a method of this invention is to administer to a subject in need of treatment an effective amount of a substituted dialkyl ether, substituted aryl-alkyl ether, substituted dialkyl thioether, substituted dialkyl ketone, or substituted-alkyl compound, or a pharmaceutically acceptable salt thereof, or a combination comprising said compound and another therapeutically active agent such as a COX-2 inhibitor.

### SUMMARY OF THE INVENTION

This invention relates to methods, compositions, and combinations using or having substituted dialkyl ethers, substituted dialkyl thioethers, substituted dialkyl ketones, or substituted-alkyl compounds for preventing or treating osteoarthritis (“OA”), preventing or inhibiting cartilage damage, preventing or treating rheumatoid arthritis (“RA”), improving joint function, alleviating pain, including joint pain, and the like in a patient in need thereof.

One aspect of this invention is a method of inhibiting cartilage damage in a mammal, comprising administering to the mammal a therapeutically effective amount of a substituted dialkyl ether, substituted aryl-alkyl ether, substituted dialkyl thioether, substituted dialkyl ketone, or substituted-alkyl compound, or a pharmaceutically acceptable salt thereof.

Another aspect of this invention is a method of preventing cartilage damage in a mammal, comprising administering to the mammal a therapeutically effective amount of a substituted dialkyl ether, substituted aryl-alkyl ether, substituted dialkyl thioether, substituted dialkyl ketone, or substituted-alkyl compound, or a pharmaceutically acceptable salt thereof.

Another aspect of this invention is a method of preventing osteoarthritis in a mammal, comprising administering to the mammal an osteoarthritis preventing effective amount of a substituted dialkyl ether, substituted aryl-alkyl ether, substituted dialkyl thioether, substituted dialkyl ketone, or substituted-alkyl compound, or a pharmaceutically acceptable salt thereof.

Another aspect of this invention is a method of treating osteoarthritis in a mammal, comprising administering to the mammal an osteoarthritis treating effective amount of a substituted dialkyl ether, substituted aryl-alkyl ether, substituted dialkyl thioether, substituted dialkyl ketone, or substituted-alkyl compound, or a pharmaceutically acceptable salt thereof.

Another aspect of this invention is a method of preventing rheumatoid arthritis in a mammal, comprising administering to the mammal a rheumatoid arthritis preventing effective amount of a substituted dialkyl ether, substituted aryl-alkyl ether, substituted dialkyl thioether, substituted dialkyl ketone, or substituted-alkyl compound, or a pharmaceutically acceptable salt thereof.

Another aspect of this invention is a method of treating rheumatoid arthritis in a mammal, comprising administering to the mammal a rheumatoid arthritis treating effective amount of a substituted dialkyl ether, substituted aryl-alkyl ether, substituted dialkyl thioether, substituted dialkyl ketone, or substituted-alkyl compound, or a pharmaceutically acceptable salt thereof.

Another aspect of this invention is a method of treating joint inflammation in a mammal, comprising administering to the mammal a therapeutically effective amount of a substituted dialkyl ether, substituted aryl-alkyl ether, substituted dialkyl thioether, substituted dialkyl ketone, or substituted-alkyl compound, or a pharmaceutically acceptable salt thereof.

In one aspect of this invention, the joint inflammation is rheumatoid arthritic joint inflammation.

Another aspect of this invention is a method of improving joint function in a mammal, comprising administering to the mammal a joint function improving effective amount of a substituted dialkyl ether, substituted aryl-alkyl ether, substituted dialkyl thioether, substituted dialkyl ketone, or substituted-alkyl compound, or a pharmaceutically acceptable salt thereof.

Another aspect of this invention is a method of alleviating pain in a mammal, comprising administering to the mammal a pain alleviating effective amount of a substituted dialkyl ether, substituted aryl-alkyl ether, substituted dialkyl thioether, substituted dialkyl ketone, or substituted-alkyl compound, or a pharmaceutically acceptable salt thereof.

Another aspect of this invention is a method of treating systemic lupus erythematous in a mammal, comprising administering to the mammal a therapeutically effective amount of a substituted dialkyl ether, substituted aryl-alkyl ether, substituted dialkyl thioether, substituted dialkyl ketone, or substituted-alkyl compound, or a pharmaceutically acceptable salt thereof.

Another aspect of this invention is a method of treating mixed connective tissue disease in a mammal, comprising administering to the mammal a therapeutically effective amount of a substituted dialkyl ether, substituted aryl-alkyl ether, substituted dialkyl thioether, substituted dialkyl ketone, or substituted-alkyl compound, or a pharmaceutically acceptable salt thereof.

Another aspect of this invention is a method of treating an IL-6 mediated disease in a mammal, comprising administering to the mammal a therapeutically effective amount of a substituted dialkyl ether, substituted aryl-alkyl ether, substituted dialkyl thioether, substituted dialkyl ketone, or substituted-alkyl compound, or a pharmaceutically acceptable salt thereof.

Another aspect of this invention is a method of treating an IL-6 receptor mediated disease in a mammal, comprising administering to the mammal a therapeutically effective amount of a substituted dialkyl ether, substituted aryl-alkyl ether, substituted dialkyl thioether, substituted dialkyl ketone, or substituted-alkyl compound, or a pharmaceutically acceptable salt thereof.

In another aspect of this invention, the IL-6 or IL-6 receptor mediated disease is sepsis.

Another aspect is any one of the above invention methods wherein the substituted dialkyl ether is a compound of Formula I

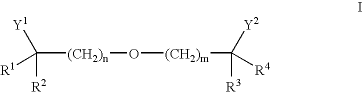

or a pharmaceutically acceptable salt thereof,

wherein:

n and m independently are integers of from 2 to 9;

R¹, R², R³, and R⁴ independently are C₁-C₆ alkyl, C₂-C₆ alkenyl, or C₂-C₆alkynyl; or

R¹ and R² together with the carbon atom to which they are attached, or R³ and R⁴ together with the carbon atom to which they are attached, or R¹ and R² together with the carbon atom to which they are attached and R³ and R⁴ together with the carbon atom to which they are attached, can complete a carbocyclic ring having from 3 to 6 carbons;

Y¹ and Y² independently are COOH, CHO, tetrazole, or COOR⁵, wherein R⁵ is C₁-C₆ alkyl, C₂-C₆ alkenyl, or C₂-C₆ alkynyl; and

wherein the alkyl, alkenyl, and alkynyl groups may be substituted with one or two groups selected from halo, hydroxy, C₁-C₆ alkoxy, and phenyl;

wherein halo includes chloro, bromo, and iodo, C₁-C₆ alkoxy is a C₁-C₆alkyl group linked through oxygen.

Another aspect is any one of the above invention methods wherein the substituted dialkyl ether is a compound named 6-(5-carboxy-5-methyl-hexyloxy)-2,2-dimethyl-hexanoic acid, or a pharmaceutically acceptable salt thereof.

Another aspect is any one of the above invention methods wherein the substituted dialkyl ether is a compound named 6-(5-carboxy-5-methyl-hexyloxy)-2,2-dimethyl-hexanoic acid, calcium salt.

Another aspect is any one of the above invention methods wherein the substituted dialkyl ether is a compound named 6-(5-carboxy-5-methyl-hexyloxy)-2,2-dimethyl-hexanoic acid, calcium salt hydrate.

Another aspect is any one of the above invention methods wherein what is administered is the substituted dialkyl ether that is Crystal Form 1 of a compound named 6-(5-carboxy-5-methyl-hexyloxy)-2,2-dimethyl-hexanoic acid, calcium salt.

Another aspect is any one of the above invention methods wherein what is administered is the substituted dialkyl ether that is Crystal Form 2 of a compound named 6-(5-carboxy-5-methyl-hexyloxy)-2,2-dimethyl-hexanoic acid, calcium salt.

Another aspect is any one of the above invention methods wherein the substituted dialkyl ether is a compound named 6-(5-carboxy-5-methyl-hexyloxy)-2,2-dimethyl-hexanoic acid, calcium salt ethyl alcohol solvate.

Another aspect is any one of the above invention methods wherein the substituted dialkyl ether is a compound named 6-(5-carboxy-5-methyl-hexyloxy)-2,2-dimethyl-hekanoic acid, calcium salt methanol solvate.

Another aspect is any one of the above invention methods wherein the substituted dialkyl ether is a compound named 6-(5-carboxy-5-methyl-hexyloxy)-2,2-dimethyl-hexanoic acid, calcium salt 1-propyl alcohol solvate.

Another aspect is any one of the above invention methods wherein the substituted dialkyl ether is a compound named 6-(5-carboxy-5-methyl-hexyloxy)-2,2-dimethyl-hexanoic acid, calcium salt 2-propyl alcohol solvate.

Another aspect is any one of the above invention methods wherein the substituted dialkyl ether is a compound named 6-(5-carboxy-5-methyl-hexyloxy)-2,2-dimethyl-hexanoic acid, calcium salt 1-butyl alcohol solvate.

Another aspect of this invention is a method of inhibiting cartilage damage in a mammal, comprising administering to the mammal a cartilage damage inhibiting effective amount of a compound named 6-(5-carboxy-5-methyl-hexyloxy)-2,2-dimethyl-hexanoic acid, calcium salt.

Another aspect of this invention is a method of preventing cartilage damage in a mammal, comprising administering to the mammal a cartilage damage preventing effective amount of a compound named 6-(5-carboxy-5-methyl-hexyloxy)-2,2-dimethyl-hexanoic acid, calcium salt.

Another aspect of this invention is a method of preventing osteoarthritis in a mammal, comprising administering to the mammal an osteoarthritis preventing effective amount of a compound named 6-(5-carboxy-5-methyl-hexyloxy)-2,2-dimethyl-hexanoic acid, calcium salt.

Another aspect of this invention is a method of treating osteoarthritis in a mammal, comprising administering to the mammal an osteoarthritis treating effective amount of a compound named 6-(5-carboxy-5-methyl-hexyloxy)-2,2-dimethyl-hexanoic acid, calcium salt.

Another aspect of this invention is a method of preventing rheumatoid arthritis in a mammal, comprising administering to the mammal an osteoarthritis preventing effective amount of a compound named 6-(5-carboxy-5-methyl-hexyloxy)-2,2-dimethyl-hexanoic acid, calcium salt.

Another aspect of this invention is a method of treating rheumatoid arthritis in a mammal, comprising administering to the mammal an osteoarthritis treating effective amount of a compound named 6-(5-carboxy-5-methyl-hexyloxy)-2,2-dimethyl-hexanoic acid, calcium salt.

Another aspect of this invention is a method of alleviating pain in a mammal, comprising administering to the mammal a pain alleviating effective amount of a compound named 6-(5-carboxy-5-methyl-hexyloxy)-2,2-dimethyl-hexanoic acid, calcium salt.

In another aspect, a compound of Formula I that may be utilized in an invention method is selected from:

7,7′-oxybis(2,2-dimethylheptanoic acid);

5,5′-oxybis(2,2-dimethylpentanoic acid);

4,4′-oxybis(2,2-dimethylbutanoic acid);

8,8′-oxybis(2,2-dimethyloctanoic acid);

Ethyl 2,2-dimethyl-5-(4-methyl-4-ethoxycarbonylpentyloxy)pentanoate;

Ethyl 2,2-dimethyl-6-(5-methyl-5-ethoxycarbonylhexyloxy)hexanoate;

Methyl 2,2-dimethyl-8-(7-methyl-7-methoxycarbonyloctyloxy)octanoate; and

7-(4-methyl-4-hydroxycarbonylpentyloxy)-2,2-dimethylheptanoic acid;

or a pharmaceutically acceptable salt thereof.

In another aspect, a compound of Formula I that may be utilized in an invention method is selected from:

5-(3-Carboxy-3-methyl-butoxy)-2,2-dimethyl-pentanoic acid;

2,2-Diethyl-5-(4-methoxycarbonyl-4-methyl-pentyloxy)-pentanoic acid;

6-(3-Carboxy-3-ethyl-4-methyl-pentyloxy)-2,2-diethyl-hexanoic acid methyl ester;

2-(3-Chloro-propyl)-5-(5-formyl-7-hydroxy-5-methyl-heptyloxy)-2-methyl-pentanoic acid;

6-(5-Carboxy-5-methyl-hexyloxy)-2,2-dimethyl-hexanoic acid;

6-(5-Carboxy-5-ethyl-heptyloxy)-2,2-diethyl-hexanoic acid, bis sodium salt;

6-(5-Butyl-5-methoxycarbonyl-nonyloxy)-2-ethyl-2-methyl-hexanoic acid;

6-(5-Ethoxycarbonyl-6-hydroxy-5-hydroxymethyl-hexyloxy)-2,2-bis-hydroxymethyl-hexanoic acid ethyl ester;

2,2-Dipropyl-6-[5-propyl-5-(1H-tetrazol-5-yl)-octyloxy]-hexanal;

1-{4-[4-(1-Carboxycyclopropan-1-yl)-butyloxy]-butyl}-cyclopropanecarboxylic acid;

1-[4-(5,5-Dimethyl-6-oxo-hexyloxy)-butyl]-cyclopentanecarbaldehyde;

2-Benzyl-6-(5,5-dimethyl-6-oxo-hexyloxy)-2-methyl-hexanal:

6-(6-Ethyl-6-formyl-octyloxy)-2,2-dimethyl-hexanoic acid:

7-(5-Carboxy-5-ethyl-6-methyl-heptyloxy)-2-ethyl-2-isobutyl-heptanoic acid;

2-[2-(6-Carboxy-6-hexyl-dodecyloxy)-ethyl]-2-hexyl-octanoic acid:

8-(3-Carboxy-3-isobutyl-5-methyl-hexyloxy)-2,2-dipropyl-octanoic acid, bis potassium salt:

8-(4-Carboxy-4-methyl-pentyloxy)-2,2-diethyl-octanoic acid:

2-Bromomethyl-9-(4-carboxy-4-chloromethyl-5-hydroxy-pentyloxy)-2-iodomethyl-nonanoic acid:

9-(5-Carboxy-5-pentyl-decyloxy)-2,2-bis-methoxymethyl-nonanoic acid, 1:1 salt with triethylamine:

10-(5,5-Dimethyl-6-oxo-hexyloxy)-2,2-dimethyl-decanoic acid:

11-(5-Hexyloxycarbonyl-5-methyl-hexyloxy)-2,2-dimethyl-undecanoic acid ethyl ester;

5-{3-Ethyl-1-[6-Ethyl-6-(1H-tetrazol-5-yl)-octan-1-yloxy]-undecan-3-yl}-tetrazole; and

11-(10-Benzyl-10-carboxy-11-chloro-undecyloxy)-2,2-diethyl-undecanoic acid;

or a pharmaceutically acceptable salt thereof.

Another aspect is any one of the above invention methods wherein the substituted-alkyl compound is a compound of Formula II

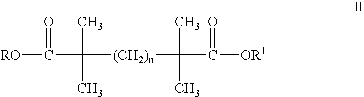

or a pharmaceutically acceptable salt thereof,

wherein n is 6, 7, 8, 9, or 10; and

R and R¹ are selected from the group consisting of hydrogen and C₁-C₈alkyl.

In another aspect, a compound of Formula II that may be utilized in an invention method is selected from:

2,2,9,9-tetramethyldecanedioic acid; and

2,2,12,12-tetramethyltridecanedioic acid;

or a pharmaceutically acceptable salt thereof.

Another aspect is any one of the above invention methods wherein the substituted-alkyl compound is a compound of Formula III

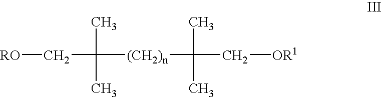

or a pharmaceutically acceptable salt thereof,

wherein n is 6, 7, 8, 9, or 10;

R and R¹ are selected from the group consisting of hydrogen, (C₁-C₁₂alkyl)-C(═O)—, HO₂C(CH₂)ₘ—CH₂—C(═O)—, phenyl-CH₂—C(H)(NH₂)—C(═O)—, and (HO)₂—P(═O)—; and

m is an integer of from 1 to 3; wherein alkyl is straight or branched.

In another aspect, a compound of Formula III that may be utilized in an invention method is selected from:

2,2,9,9-tetramethyl-1,10-decanediol;

or a pharmaceutically acceptable salt thereof.

Another aspect is any one of the above invention methods wherein the substituted aryl-alkyl ether is a compound of Formula IV

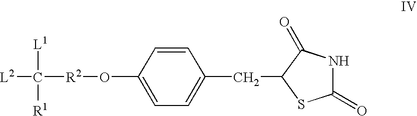

or a pharmaceutically acceptable salt thereof,

wherein

R¹ is C₁-C₁₀ alkyl, C₃-C₇ cycloalkyl, phenyl-(C₁-C₅ alkyl)-, phenyl, thienyl, furanyl, thiazolyl, pyridinyl, or R³R⁴N—;

R³ and R⁴ are the same or different C₁-C₄ alkyl, or R³ and R⁴ are combined to each other either directly, or as interrupted by a heteroatom selected from N, O, and S, with the nitrogen atom to which they are both bonded to form a 5- or 6-membered ring, wherein the 5- or 6-membered ring is piperidinyl, morpholinyl, pyrrolidinyl, or piperazinyl;

R² is a bond or —(CH₂)ₘ—;

L¹ and L² are the same or different C₁-C₄ alkyl, or L¹ and L² are combined to each other to form —(CH₂)ₚ—;

p is an integer of from 2 to 6; and

when R¹ is C₃-C₇ cycloalkyl, phenyl-(C₁-C₅ alkyl)-, phenyl, thienyl, furanyl, thiazolyl, pyridinyl, or R³R⁴N—, L¹ and L² may further by hydrogen;

wherein the C₃-C₇ cycloalkyl, phenyl-(C₁-C₅ alkyl)-, phenyl, thienyl, furanyl, thiazolyl, pyridinyl, piperidinyl, morpholinyl, pyrrolidinyl, and piperazinyl groups may optionally have from 1 to 3 substituents independently selected from C₁-C₄ alkyl, (C₁-C₄alkyl)-O—, F, Cl, Br, I, OH, and a methylenedioxy group of formula —O—(CH₂)ₘ—O—, wherein the oxygen atoms of the methylenedioxy group are bonded to contiguous carbon atoms to form a ring of from 5 to 7 members; and

Each m independently is an integer of from 1 to 3.

In another aspect, a compound of Formula IV that may be utilized in an invention method is selected from:

5-[4-(1-methylcyclohexylmethyloxy)benzyl]thiazolidine-2,4-dione;

a compound of any one of Examples 1 to 8, 10, and 11 of U.S. Pat. No. 4,287,200;

any one of Compound Nos. 1 to 54 of Example 10 of U.S. Pat. No. 4,287,200; and

any one of Compound Nos. 1 to 7 of Example 12 of U.S. Pat. No. 4,287,200;

or a pharmaceutically acceptable salt thereof.

Another aspect is any one of the above invention methods wherein the substituted dialkyl ether, substituted aryl-alkyl ether, substituted dialkyl thioether, substituted dialkyl ketone, or substituted-alkyl compound is a compound of Formula V

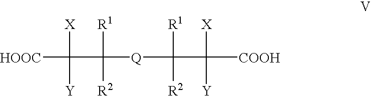

or a pharmaceutically acceptable salt thereof, or an in vivo hydrolyzable functional derivative selected from an ester, amide, or anhydride with (C₁-C₅ alkyl)-COOH;

wherein

R¹ and R² each independently represent an unsubstituted or substituted hydrocarbyl selected from C₁-C₆ alkyl optionally substituted by phenyl, OH, (C₁-C₆ alkyl)-O—, F, Cl, or Br, C₂-C₆ alkenyl, C₂-C₆alkynyl, C₃-C₇ cycloalkyl, phenyl optionally substituted by OH, (C₁-C₆ alkyl)-O—, C₁-C₆ alkyl, F, Cl, or Br, or heterocyclyl;

X and Y each independently represent hydrogen, C₁-C₆ alkyl, F, Cl, Br, COOH, (C₁-C₆ alkyl)-O—C(═O)—, or (C₁-C₆ alkyl)-N(H)—C(═O)—, and further one of X and Y can also be (C₁-C₆ alkyl)-O—, HO, or NC—;

Q represents a diradical consisting of an alkylenyl diradical of from 8 to 14 carbon atoms or a heteroalkylenyl diradical of from 8 to 14 members having carbon atoms and a heteroatom selected from S, S(O), S(O)₂, N(H), N(C₁-C₆ alkyl), N(CH₂-phenyl), and 0, wherein the alkylenyl or heteroalkylenyl may optionally be substituted by oxo (═O), F, Cl, Br, OH, or (C₁-C₆ alkyl)-O—, and wherein any from 1 to 4 contiguous atoms in the alkylenyl or heteroalkylenyl may comprise a C₃-C₇ cycloalkyl and wherein any from 2 to 4 contiguous atoms in the alkylenyl or heteroalkylenyl may comprise a phenyl.

In another aspect, a compound of Formula V that may be utilized in an invention method is selected from:

2,3,3,14,14,15-hexamethyl-hexadecane-1,16-dioic acid;

2,15-di-carbamoyl-3,3,14,14-tetramethyl-hexadecane-1,16-dioic acid;

3,14-diethyl-3,14-dimethyl-hexadecane-1,16-dioic acid;

3,3,14,14-tetra-(2-propenyl)-hexadecane-1,16-dioic acid;

3,3,14,14-tetra-cyclohexyl-hexadecane-1,16-dioic acid;

2,15-dibromo-3,3,14,14-tetraphenyl-hexadecane-1,16-dioic acid;

1,2-cyclopropylidine-bis-(3,3-dimethyl-7-yl-heptanoic acid);

9,9-pentamethylene-3,3-15,15-tetramethyl-heptadecane-1,17-dioic acid;

1,2-cyclohexylidene-bis-(3,3-dimethyl-7-yl-heptanoic acid);

1,2-phenylene-(3,3-dimethyl-7-yl-heptanoic acid);

3,3,15,15-tetramethyl-9-thia-heptadecane-1,17-dioic acid;

9-oxa-3,3,15,15-tetramethyl-heptadecane-1,17-dioic acid;

9-aza-3,3,15,15-tetramethyl-heptadecane-1,17-dioic acid;

3,3,14,14-tetramethyl-6,11-dithiahexadecane-1,16-dioic acid;

2,15-difluoro-3,3,14,14-tetramethyl-hexadecane-1,16-dioic acid;

2,2,15,15-tetrafluoro-3,3,14,14-tetramethyl-hexadecane-1,16-dioic acid;

2,2,15,15-tetrachloro-3,3,14,14-tetramethyl-hexadecane-1,16-dioic acid;

3,3,14,14-tetrahydroxymethyl-hexadecane-1,16-dioic acid;

2,15-dichloro-3,14-di(chloromethyl)-3,14-dimethyl-hexadecane-1,16-dioic acid;

2,15-dichloro-3,3,14,14-tetra(chloromethyl)-hexadecane-1,16-dioic acid;

3,3,14,14-tetra-(4-hydroxyphenyl)-hexadecane-1,16-dioic acid;

3,3,14,14-tetra-(4-chlorophenyl)-hexadecane-1,16-dioic acid;

3,3,14,14-tetra-(4-methyl-phenyl)-hexadecane-1,16-dioic acid; and

3,3,14,14-tetra-(4-methoxy-phenyl)-hexadecane-1,16-dioic acid;

or a pharmaceutically acceptable salt thereof.

In another aspect, a compound of Formula V that may be utilized in an invention method is selected from:

1,1,14,14-tetra(ethoxycarbonyl)-2,2,13,13-tetramethyl-tetradecane;

1,1,16,16-tetra(ethoxycarbonyl)-2,2,15,15-tetramethyl-hexadecane;

1,1,12,12-tetra(ethoxycarbonyl)-2,2,11,11-tetramethyl-dodecane;

3,3,14,14-tetramethyl-hexadecane-1,16-dioic acid;

3,3,16,16-tetramethyl-octadecane-1,18-dioic acid;

3,3,12,12-tetramethyl-tetradecane-1,14-dioic acid;

1,14-di-(ethoxycarbonyl)-1,14-dicyano-2,2 13,13-tetramethyl-tetradecane;

2,15-dicyano-3,3,14,14-tetramethyl-hexadecane-1,16-dioic acid;

2,15-dibromo-3,3,14,14-tetramethyl-hexadecane-1,16-dioic acid;

2,3,3,14,14,15-hexamethyl-hexadecane-1,16-dioic acid;

1,14-diethoxycarbonyl-2,2,13,13-tetramethyl-tetradecane;

1,14-di-(ethoxycarbonyl)-1,14-dibromo-2,2,13,13-tetramethyl-tetradecane;

1,14-bis-carbamoyl-2,2,13,13-tetramethyl-tetradecane;

2,15-dichloro-3,3,14,14-tetramethylhexadecane-1,16-dioic acid;

2,15-dibromo-3,3,14,14-tetramethylhexadecane-1,16-dioic acid;

2,15-dihydroxy-3,3,14,14-tetramethylhexadecane-1,16-dioic acid;

1,14-di-(carbomethoxy)-1,14-dibromo-2,2,13,13-tetramethyltetradecane;

1,14-di-(carbomethoxy)-1,14-dichloro-2,2,13,13-tetramethyltetradecane;

2,15-dimethoxy-3,3,14,14-tetramethylhexadecane-1,16-dioic acid;

1,1,18,18-tetra(carboethoxy)-2,2,17,17-tetramethyloctadecane;

3,3,18,18-tetramethyleicosane-1,20-dioic acid;

3,3,14,14-tetramethyl-8-hexadecene-1,16-dioic acid;

3,3,14,14-tetraphenyl-6,11-diketohexadecane-1,16-dioic acid;

3,3,14,14-tetraphenylhexadecane-1,16-dioic acid;

1,4-phenylene-bis-[(1,1-dimethyl-but-4-yl)-dipropionic acid dimethyl ester];

1,4-phenylene-bis-[(1,1-dimethyl-but-4-yl)-dipropionic acid];

1,4-phenylene-bis(3,3-dimethyl-6-yl-5-hexenoic acid methyl ester);

1,3-phenylene-bis(3,3-dimethyl-6-yl-5-hexenoic acid methyl ester);

1,4-phenylene-bis(3,3-dimethyl-6-yl-hexanoic acid methyl ester);

1,3-phenylene-bis(3,3-dimethyl-6-yl-hexanoic acid methyl ester);

1,4-phenylene-bis(3,3-dimethyl-6-yl-hexanoic acid);

1,3-phenylene-bis(3,3-dimethyl-6-yl-hexanoic acid);

1,4-(cyclohexylidene-bis-(3,3-dimethyl-6-yl-hexanoic acid methyl ester);

1,3-(cyclohexylidene-bis-(3,3-dimethyl-6-yl-hexanoic acid methyl ester);

1,4-(cyclohexylidene-bis-(3,3-dimethyl-6-yl-hexanoic acid);

1,3-(cyclohexylidene-bis-(3,3-dimethyl-6-yl-hexanoic acid);

1,4-phenylene-bis(3,3-dimethyl-7-yl-5-heptenoic acid);

1,3-phenylene-bis(3,3-dimethyl-7-yl-5-heptenoic acid);

1,4-phenylene-bis(3,3-dimethyl-7-yl-heptanoic acid);

1,3-phenylene-bis(3,3-dimethyl-7-yl-heptanoic acid);

1,4-(cyclohexylidene-bis-(3,3-dimethyl-7-yl-heptanoic acid);

1,3-(cyclohexylidene-bis-(3,3-dimethyl-7-yl-heptanoic acid); and

1,4-(cyclohexylidene-bis-(3,3-dimethyl-5-oxo-7-yl-heptanoic acid);

or a pharmaceutically acceptable salt thereof.

Another aspect is any one of the above invention methods wherein the substituted dialkyl ether, substituted aryl-alkyl ether, substituted dialkyl thioether, substituted dialkyl ketone, or substituted-alkyl compound is a compound of Formula VI

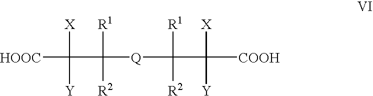

or a pharmaceutically acceptable salt thereof, or an in vivo hydrolyzable functional derivative selected from an ester, amide, or anhydride with (C₁-C₅ alkyl)-COOH;

wherein

R¹ and R² each independently represent an unsubstituted or substituted C₁-C₆ alkyl optionally substituted by OH, (C₁-C₆ alkyl)-O—, F, Cl, Br, or phenyl, wherein the phenyl optionally substituted one or more times by OH, (C₁-C₆ alkyl)-O—, C₁-C₆ alkyl, F, Cl, or Br, C₂-C₆alkenyl, C₂-C₆ alkynyl, C₃-C₇ cycloalkyl, phenyl optionally substituted by OH, (C₁-C₆ alkyl)-O—, C₁-C₆ alkyl, F, Cl, or Br, or heterocycle;

X and Y each independently represent hydrogen, C₁-C₆ alkyl, (C₁-C₆alkyl)-O—, HO, NC—, F, Cl, Br, COOH, (C₁-C₆ alkyl)-O—C(═O)—, or (C₁-C₆ alkyl)-N(H)—C(═O)—;

Q represents a diradical consisting of an alkylenyl diradical of from 8 to 14 carbon atoms or a heteroalkylenyl diradical of from 8 to 14 members having carbon atoms and a heteroatom selected from S, S(O), S(O)₂, N(H), N(C₁-C₆ alkyl), N(CH₂-phenyl), and O, wherein the alkylenyl or heteroalkylenyl may optionally be substituted by oxo (═O), F, Cl, Br, OH, or (C₁-C₆ alkyl)-O—, and wherein any from 1 to 4 contiguous atoms in the alkylenyl or heteroalkylenyl may comprise a C₃-C₇ cycloalkyl and wherein any from 2 to 4 contiguous atoms in the alkylenyl or heteroalkylenyl may comprise a phenyl.

In another aspect, a compound of Formula VI that may be utilized in an invention method is selected from:

2,15-difluoro-3,3,14,14-tetramethyl-1,16-hexadecanedioic acid;

2,15-dichloro-3,3,14,14-tetramethyl-hexadecane-1,16-dioic acid diisopropyl ester; and

2,2,15,15-tetrachloro-3,3,14,14-tetramethyl-hexadecane-1,16-dioic acid; or a pharmaceutically acceptable salt thereof.

Another aspect is any one of the above invention methods wherein the substituted dialkyl ether, substituted aryl-alkyl ether, substituted dialkyl thioether, substituted dialkyl ketone, or substituted-alkyl compound is a compound of Formula VII

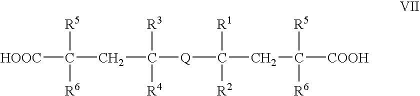

or a pharmaceutically acceptable salt thereof, or in vivo hydrolysable functional derivatives of the carboxylic groups thereof selected from C₁-C₆ alkyl ester, unsubstituted amide, C₁-C₆ alkyl amide, bis(C₁-C₆ alkyl) amide, anhydride with a C₁-C₆ carboxylic acid, and lactone formed by a dehydrative ring closure between a COOH group and any OH group of R⁵ or R⁶,

wherein:

R¹, R², R³, and R⁴ each independently represents a hydrogen, an unsubstituted or substituted hydrocarbyl radical selected from C₁-C₆ alkyl, C₂-C₆ alkenyl, C₂-C₆ alkynyl, C₃-C₇ cycloalkyl, phenyl, and phenyl-(C₁-C₃ alkylenyl), or a heterocyclyl radical;

R⁵ and R⁶ independently represent hydrogen, hydroxyl, C₁-C₆ alkyl, chloro, bromo, cyano, nitro, C₁-C₆ alkoxy, or CF₃;

Q represents a diradical consisting of an unsubstituted or substituted linear chain of 2 to 14 carbon atoms, one or more of which may be replaced by heteroatoms selected from O, S, S(O), S(O)₂, N(H), N(C₁-C₆ alkyl), and N(CH₂phenyl);

wherein substituents are selected from oxo (═O), F, Cl, Br, OH, or (C₁-C₆alkyl)-O—, and wherein any from 1 to 4 contiguous atoms in the in the linear chain may comprise a C₃-C₇ cycloalkyl and wherein any from 2 to 4 contiguous atoms in the linear chain may comprise a phenyl.

In another aspect, a compound of Formula VII that may be utilized in an invention method is the compound wherein R¹, R², R³, R⁴, R⁵, and R⁶ are not each hydrogen.

In another aspect, a compound of Formula VII that may be utilized in an invention method is selected from:

4,4,11,11-tetramethyltetradecanedioic acid;

diethyl 4,4,13,13-tetramethylhexadeca-2,5,11,14-tetraenedionate;

4,4,13,13-tetramethylhexadecanedioic acid;

4,4,15,15-tetramethyloctadecanedioic acid;

2,2,15,15-tetramethylhexadecanedioic acid; and

2,2,17,17-tetramethyloctadecanedioic acid.

In another aspect, a compound that may be utilized in an invention method is any single compound or genus of compounds of U.S. patent application Ser. No. 10/205,939; U.S. Pat. Nos. 6,410,802; 6,459,003; and 6,506,799; in U.S. patent application Publication No. U.S. Ser. No. 2003/0065195; and in PCT International Patent Application Publication No. WO 00/59855, or a pharmaceutically acceptable salt thereof.

In another aspect, a compound that may be utilized in an invention method is any single compound or genus of compounds of U.S. patent application Ser. No. 10/205,939; U.S. Pat. Nos. 6,410,802; 6,459,003; and 6,506,799; in U.S. patent application Publication No. U.S. Ser. No. 2003/0065195; and in PCT International Patent Application Publication No. WO 00/59855, or a pharmaceutically acceptable salt thereof selected from:

6-(6-Hydroxy-5,5-dimethyl-hexyloxy)-2,2-dimethyl-hexan-1-ol;

phosphoric acid mono-(1,1-dimethyl-5-(5-methyl-5-phosphonooxy-hexyloxy)-pentyl) ester sodium salt;

phosphoric acid dibenzyl ester 5-(5-(bis-benzyloxy-phosphoryloxy)-5-methyl-hexyloxy)-1,1-dimethyl-pentyl ester;

phosphoric acid mono-(1,1-dimethyl-4-(4-methyl-4-phosphonooxy-pentyloxy)-butyl) ester sodium salt;

phosphoric acid dibenzyl ester 4-(4-(bis-benzyloxy-phosphoryloxy)-4-methyl-pentyloxy)-1,1-dimethyl-butyl ester; and

6-(5-hydroxy-5-methyl-hexyloxy)-2-methyl-hexan-2-ol;

or a pharmaceutically acceptable salt thereof.

In another aspect, a compound that may be utilized in an invention method is any single compound or genus of compounds of U.S. patent application Ser. No. 09/976,867; U.S. patent application Publication No. U.S. Ser. No. 2003/0018013; and in PCT International Patent Application Publication No. WO 02/30863, or a pharmaceutically acceptable salt thereof.

In another aspect, a compound that may be utilized in an invention method is any single compound or genus of compounds of U.S. patent application Ser. No. 09/976,867; U.S. patent application Publication No. U.S. Ser. No. 2003/0018013; and in PCT International Patent Application Publication No. WO 02/30863, or a pharmaceutically acceptable salt thereof, selected from:

5-[2-(5-Hydroxy-4,4-dimethyl-pentyloxy)-ethoxy]-2,2-dimethyl-pentan-1-ol; and

3-{3-[3-(2-Carboxy-2-methyl-propyl)-phenoxy]-phenyl}-2,2-dimethyl-propionic acid; or

a pharmaceutically acceptable salt thereof.

In another aspect, a compound that may be utilized in an invention method is any single compound or genus of compounds of U.S. patent application Ser. No. 09/976,938; U.S. patent application Publication No. U.S. Ser. No. 2003/0078239 and PCT International Patent Application Publication No. WO 02/30860, or a pharmaceutically acceptable salt thereof.

In another aspect, a compound that may be utilized in an invention method is any single compound or genus of compounds of U.S. patent application Ser. No. 09/976,938; U.S. patent application Publication No. U.S. Ser. No. 2003/0078239 and PCT International Patent Application Publication No. WO 02/30860, or a pharmaceutically acceptable salt thereof selected from:

1,13-Dihydroxy-2,2,12,12-tetramethyl-tridecan-7-one;

2,2,12,12-Tetramethyl-7-oxo-tridecanedioic acid diethyl ester;

1,11-Dihydroxy-2,2,10,10-tetramethyl-undecan-6-one;

2,12-Dimethyl-7-oxo-2,12-di-p-tolyl-tridecanedioic acid;

1,13-Dihydroxy-2,12-dimethyl-2,12-di-p-tolyl-tridecan-7-one;

2,12-Bis-(4-isobutyl-phenyl)-2,12-dimethyl-7-oxo-tridecanedioic acid;

1,13-Dihydroxy-2,12-bis-(4-isobutyl-phenyl)-2,12-dimethyl-tridecan-7-one;

2,10-Dimethyl-6-oxo-2,10-diphenyl-undecanedioic acid;

1,11-Dihydroxy-2,10-dimethyl-2,10-diphenyl-undecan-6-one;

9-Hydroxy-3-(6-hydroxy-5,5-dimethyl-hexyl)-8,8-dimethylnonan-2-one;

Bis [3-(3-hydroxy-2,2-dimethylpropyl)phenyl]methanone;

3-{3-[3-(2-Carboxy-2-methyl-propyl)-benzoyl]-phenyl}-2,2-dimethyl-propanoic acid;

2,2,12,12-Tetramethyl-7-oxo-tridecanedioic acid bis-methylamide;

2,2,12,12-Tetramethyl-7-oxo-tridecanedioic acid bis-phenylamide; and

7-Oxo-2,12-dimethyl-2,12-diphenyl-tridecanedioic acid; or

a pharmaceutically acceptable salt thereof.

In another aspect, a compound that may be utilized in an invention method is any single compound or genus of compounds of U.S. patent application Ser. No. 09/976,898; U.S. patent application Publication No. U.S. Ser. No. 2002/0077316; and in PCT International Patent Application Publication No. WO 02/30884, or a pharmaceutically acceptable salt thereof.

In another aspect, a compound that may be utilized in an invention method is any single compound or genus of compounds of U.S. patent application Ser. No. 09/976,898; U.S. patent application Publication No. U.S. Ser. No. 2002/0077316; and in PCT International Patent Application Publication No. WO 02/30884, or a pharmaceutically acceptable salt thereof selected from:

5-[2-(4-Carboxy-4-methyl-pentylsulfanyl)-ethylsulfanyl]-2,2-dimethyl-pentanoic acid;

Bis-(5,5-dimethyl-6-tetrahydropyranyloxy-hexyl)-sulfide;

6-(5,5-Dimethyl-6-hydroxy-hexyl-sulfanyl)-2,2-dimethyl-hexan-1′-ol;

Ethyl 5-mercapto-2,2-dimethylpentanoate;

2,2,12,12-Tetramethyl-6,8-dithiatridecane-1,13-dioic acid;

6-(6-Hydroxy-5-methyl-5-phenylhexylsulfanyl)-2-methyl-2-phenylhexan-1-ol;

6-(5-Carboxy-5-phenylhexylsulfanyl)-2-methyl-2-phenyl-hexanoic acid;

Di-(6-hydroxy-5,5-dimethylpentyl)sulfide;

5-(5-Hydroxy-4-methyl-4-phenylpentylsulfanyl)-2-methyl-2-phenylpentan-1-ol; and

2,2,12,12-Tetramethyl-5,9-dithiatridecanedioic acids disodium salt; or

a pharmaceutically acceptable salt thereof.

In another aspect, a compound that may be utilized in an invention method is any single compound or genus of compounds of U.S. patent application Ser. No. 09/976,899; U.S. patent application Publication No. U.S. Ser. No. 2003/0022865; and in PCT International Patent Application Publication No. WO 02/30882, or a pharmaceutically acceptable salt thereof.

In another aspect, a compound that may be utilized in an invention method is any single compound or genus of compounds of U.S. patent application Ser. No. 09/976,899; U.S. patent application Publication No. U.S. Ser. No. 2003/0022865; and in PCT International Patent Application Publication No. WO 02/30882, or a pharmaceutically acceptable salt thereof selected from:

6-(5,5-Dimethyl-6-hydroxy-hexane-1-sulfinyl)-2,2-dimethyl-hexan-1-ol;

6-(6-Hydroxy-5-methyl-5-phenylhexylsulfinyl)-2-methyl-2-phenylhexan-1-ol;

5-(5-Hydroxy-4,4-dimethyl-pentyl-1-sulfinyl)-2,2-dimethyl-pentan-1-ol; and

5-(5-Hydroxy-4-methyl-4-phenylpentylsulfinyl)-2-methyl-2-phenylpentan-1-ol; or

a pharmaceutically acceptable salt thereof.

Another aspect of this invention is a method of inhibiting cartilage damage in a mammal, comprising administering to the mammal a therapeutically effective amount of a means for inhibiting cartilage damage in a mammal in admixture with a pharmaceutically acceptable carrier, diluent, or excipient.

Another aspect of this invention is a method of preventing cartilage damage in a mammal, comprising administering to the mammal a therapeutically effective amount of a means for preventing cartilage damage in a mammal in admixture with a pharmaceutically acceptable carrier, diluent, or excipient.

Another aspect of this invention is a method of preventing osteoarthritis in a mammal, comprising administering to the mammal an osteoarthritis preventing effective amount of a means for preventing osteoarthritis in a mammal in admixture with a pharmaceutically acceptable carrier, diluent, or excipient one.

Another aspect of this invention is a method of treating osteoarthritis in a mammal, comprising administering to the mammal an osteoarthritis treating effective amount of a means for treating osteoarthritis in a mammal in admixture with a pharmaceutically acceptable carrier, diluent, or excipient.

Another aspect of this invention is a method of preventing rheumatoid arthritis in a mammal, comprising administering to the mammal a rheumatoid arthritis preventing effective amount of a means for preventing rheumatoid arthritis in a mammal in admixture with a pharmaceutically acceptable carrier, diluent, or excipient

Another aspect of this invention is a method of treating rheumatoid arthritis in a mammal, comprising administering to the mammal a rheumatoid arthritis treating effective amount of a means for treating rheumatoid arthritis in a mammal in admixture with a pharmaceutically acceptable carrier, diluent, or excipient.

Another aspect of this invention is a method of improving joint function in a mammal, comprising administering to the mammal a joint function improving effective amount of a means for improving joint function in a mammal in admixture with a pharmaceutically acceptable carrier, diluent, or excipient.

Another aspect of this invention is a method of treating systemic lupus erythematous in a mammal, comprising administering to the mammal a therapeutically effective amount of a means for treating systemic lupus erythematous in a mammal in admixture with a pharmaceutically acceptable carrier, diluent, or excipient.

Another aspect of this invention is a method of treating mixed connective tissue disease in a mammal, comprising administering to the mammal a therapeutically effective amount of a means for treating mixed connective tissue disease in a mammal in admixture with a pharmaceutically acceptable carrier, diluent, or excipient.

Another aspect of this invention is a method of treating an IL-6 mediated disease in a mammal, comprising administering to the mammal a therapeutically effective amount of a means for treating an IL-6 mediated disease in a mammal in admixture with a pharmaceutically acceptable carrier, diluent, or excipient.

Another aspect of this invention is a method of treating an IL-6 receptor mediated disease in a mammal, comprising administering to the mammal a therapeutically effective amount of a means for treating an IL-6 receptor mediated disease in a mammal in admixture with a pharmaceutically acceptable carrier, diluent, or excipient.

Another aspect of this invention is a method of treating sepsis in a mammal, comprising administering to the mammal a therapeutically effective amount of a means for treating sepsis in a mammal in admixture with a pharmaceutically acceptable carrier, diluent, or excipient.

Another aspect of this invention is a method of alleviating pain in a mammal, comprising administering to the mammal a pain alleviating effective amount of a means for alleviating pain in a mammal in admixture with a pharmaceutically acceptable carrier, diluent, or excipient.

Another aspect is any one of the above methods of alleviating pain, wherein the pain is osteoarthritic pain.

Another aspect is any one of the above methods of alleviating pain, wherein the pain is rheumatoid arthritic pain.

Another aspect is any one of the above methods of alleviating pain, wherein the pain is joint pain.

Another aspect is any one of the above methods of alleviating pain, wherein the pain is osteoarthritic joint pain.

Another aspect is any one of the above methods of alleviating pain, wherein the pain is rheumatoid arthritic joint pain.

Another aspect is any one of the above methods of alleviating pain, wherein the pain is acute pain.

Another aspect is any one of the above methods of alleviating pain, wherein the pain is acute joint pain.

Another aspect is any one of the above methods of alleviating pain, wherein the pain is chronic pain.

Another aspect is any one of the above methods of alleviating pain, wherein the pain is chronic joint pain.

Another aspect is any one of the above methods of alleviating pain, wherein the pain is inflammatory pain.

Another aspect is any one of the above methods of alleviating pain, wherein the pain is inflammatory joint pain.

Another aspect is any one of the above methods of alleviating pain, wherein the pain is a mechanical pain.

Another aspect is any one of the above methods of alleviating pain, wherein the pain is a mechanical joint pain.

Another aspect is any one of the above methods of alleviating pain, wherein the pain is mediated by IL-6, IL-6sR, or IL-6 receptor.

Another aspect is any one of the above methods of alleviating pain other than joint pain, osteoarthritic pain, rheumatoid arthritic pain, and inflammatory joint pain, wherein the pain is pain mediated by IL-6, IL-6sR, or IL-6 receptor.

Another aspect of this invention is any one of the above methods of alleviating pain, wherein the pain is mediated by a protein or protein and its receptor selected from: oncostatin-M, oncostatin-M and oncostatin-M receptor, leukemia inhibitor factor (“LIF”), LIF and leukemia inhibitor factor receptor (“LIFR”), interleukin-11 (“IL-11”), and IL-11 and interleukin-11 receptor (“IL-11R”).

Another aspect is any one of the above methods of alleviating pain other than joint pain, osteoarthritic pain, rheumatoid arthritic pain, and inflammatory joint pain, wherein the pain is pain mediated by endothelin-1.

Another aspect is any one of the above methods of alleviating pain, wherein the pain is bone cancer pain.

Another aspect is any one of the above methods of alleviating pain, wherein the pain is neuropathic pain.

Another aspect is any one of the above methods of alleviating pain, wherein the pain is static allodynia.

Another aspect is any one of the above methods of alleviating pain, wherein the pain is dynamic allodynia.

Another aspect is any one of the above methods of alleviating pain, wherein the pain is headache pain.

Another aspect of this invention is a pharmaceutical composition, comprising an amount of a substituted dialkyl ether, substituted aryl-alkyl ether, substituted dialkyl thioether, substituted dialkyl ketone, or substituted-alkyl compound, or a pharmaceutically acceptable salt thereof, effective for use in any one of the above methods, and a pharmaceutically acceptable carrier, diluent, or excipient.

Another aspect of this invention is use of a pharmaceutical composition, comprising an amount of a substituted dialkyl ether, substituted aryl-alkyl ether, substituted dialkyl thioether, substituted dialkyl ketone, or substituted-alkyl compound, or a pharmaceutically acceptable salt thereof, effective for use in any one of the above methods, and a pharmaceutically acceptable carrier, diluent, or excipient.

Another aspect of this invention is use of a substituted dialkyl ether, substituted aryl-alkyl ether, substituted dialkyl thioether, substituted dialkyl ketone, or substituted-alkyl compound, or a pharmaceutically acceptable salt thereof, in the preparation of a medicament effective for use in any one of the above methods or uses.

Another aspect of this invention is a combination, comprising a selective COX-2 inhibitor, or a pharmaceutically acceptable salt thereof, and a substituted dialkyl ether, substituted aryl-alkyl ether, substituted dialkyl thioether, substituted dialkyl ketone, or substituted-alkyl compound, or a pharmaceutically acceptable salt thereof.

Another aspect of this invention is the combination, comprising a selective COX-2 inhibitor selected from: celecoxib, valdecoxib, and parecoxib, and a compound named 6-(5-carboxy-5-methyl-hexyloxy)-2,2-dimethyl-hexanoic acid, calcium salt.

Another aspect of this invention is a combination, comprising methotrexate and a substituted dialkyl ether, substituted aryl-alkyl ether, substituted dialkyl thioether, substituted dialkyl ketone, or substituted-alkyl compound, or a pharmaceutically acceptable salt thereof.

Another aspect of this invention is a combination, comprising a therapeutic biological agent and a substituted dialkyl ether, substituted aryl-alkyl ether, substituted dialkyl thioether, substituted dialkyl ketone, or substituted-alkyl compound, or a pharmaceutically acceptable salt thereof.

Another aspect of this invention is any invention method wherein the substituted dialkyl ether, substituted aryl-alkyl ether, substituted dialkyl thioether, substituted dialkyl ketone, or substituted-alkyl compound is replaced with an invention combination comprising the substituted dialkyl ether, substituted aryl-alkyl ether, substituted dialkyl thioether, substituted dialkyl ketone, or substituted-alkyl compound and another therapeutically active compound or therapeutic biological agent.

Another aspect of this invention is a pharmaceutical composition, comprising an amount of an invention combination effective for use in any one of the above methods, and a pharmaceutically acceptable carrier, diluent, or excipient.

Another aspect of this invention is use of a pharmaceutical composition, comprising an amount of an invention combination effective for use in any one of the above methods, and a pharmaceutically acceptable carrier, diluent, or excipient.

### FORMULATION EXAMPLE 1

Tablet Formulation:

| Ingredient | Amount (mg) |
|---|---|
| Substituted dialkyl ether, substituted aryl-alkyl ether, | 25 |
| substituted dialkyl thioether, substituted dialkyl ketone, | |
| or substituted-alkyl compound useful in the invention | |
| method | |
| Lactose | 50 |
| Cornstarch (for mix) | 10 |
| Cornstarch (paste) | 10 |
| Magnesium stearate (1%) | 5 |
| Total | 100 |

The substituted dialkyl ether, substituted aryl-alkyl ether, substituted dialkyl thioether, substituted dialkyl ketone, or substituted-alkyl compound useful in the invention method, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for inhibiting cartilage damage, improving joint function, treating rheumatoid arthritis, or treating osteoarthritis.

### FORMULATION EXAMPLE 2

Coated Tablets:

The tablets of Formulation Example 1 are coated in a customary manner with a coating of sucrose, potato starch, talc, tragacanth, and colorant.

### FORMULATION EXAMPLE 3

Injection Vials:

The pH of a solution of 500 g of 6-(5-carboxy-5-methyl-hexyloxy)-2,2-dimethyl-hexanoic acid, calcium salt, and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 25 mg of 6-(5-carboxy-5-methyl-hexyloxy)-2,2-dimethyl-hexanoic acid, calcium salt.

### FORMULATION EXAMPLE 4

Suppositories:

A mixture of 25 g of 6-(5-carboxy-5-methyl-hexyloxy)-2,2-dimethyl-hexanoic acid, calcium salt, 100 g of soya lecithin, and 1400 g of cocoa butter is fused, poured into molds, and allowed to cool. Each suppository contains 25 mg of 6-(5-carboxy-5-methyl-hexyloxy)-2,2-dimethyl-hexanoic acid, calcium salt.

### FORMULATION EXAMPLE 5

Solution:

A solution is prepared from 1 g of 6-(5-carboxy-5-methyl-hexyloxy)-2,2-dimethyl-hexanoic acid, calcium salt, 9.38 g of NaH₂PO₄.12H₂O, 28.48 g of Na₂HPO₄.12H₂O, and 0.1 g benzalkonium chloride in 940 mL of double-distilled water. The pH of the solution is adjusted to pH 6.8 using 2 M hydrochloric acid. The solution is diluted to 1.0 L with double-distilled water, and sterilized by irradiation. A 25 mL volume of the solution contains 25 mg of 6-(5-carboxy-5-methyl-hexyloxy)-2,2-dimethyl-hexanoic acid, calcium salt.

### FORMULATION EXAMPLE 6

Ointment:

500 mg of 6-(5-carboxy-5-methyl-hexyloxy)-2,2-dimethyl-hexanoic acid, calcium salt is mixed with 99.5 g of petroleum jelly under aseptic conditions. A 5 g portion of the ointment contains 25 mg of 6-(5-carboxy-5-methyl-hexyloxy)-2,2-dimethyl-hexanoic acid, calcium salt.

### FORMULATION EXAMPLE 7

Capsules:

2 kg of 6-(5-carboxy-5-methyl-hexyloxy)-2,2-dimethyl-hexanoic acid, calcium salt are filled into hard gelatin capsules in a customary manner such that each capsule contains 25 mg of 6-(5-carboxy-5-methyl-hexyloxy)-2,2-dimethyl-hexanoic acid, calcium salt.

### FORMULATION EXAMPLE 8

Ampoules:

A solution of 2.5 kg of 6-(5-carboxy-5-methyl-hexyloxy)-2,2-dimethyl-hexanoic acid, calcium salt is dissolved in 60 L of double-distilled water. The solution is sterile filtered, and the filtrate is filled into ampoules. The ampoules are lyophilized under sterile conditions and aseptically sealed. Each ampoule contains 25 mg of 6-(5-carboxy-5-methyl-hexyloxy)-2,2-dimethyl-hexanoic acid, calcium salt.

### FORMULATION EXAMPLE 9

Tablet Formulation:

| Ingredient | Amount (mg) |
|---|---|
| 6-(5-carboxy-5-methyl-hexyloxy)-2,2-dimethyl- | 25 |
| hexanoic acid, calcium salt | |
| Valdecoxib | 20 |
| Lactose | 50 |
| Cornstarch (for mix) | 10 |
| Cornstarch (paste) | 10 |
| Magnesium stearate (1%) | 5 |
| Total | 120 |

6-(5-carboxy-5-methyl-hexyloxy)-2,2-dimethyl-hexanoic acid, calcium salt, valdecoxib, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of one of the above-listed diseases, including rheumatoid arthritis.

### FORMULATION EXAMPLE 10

Coated Tablets:

The tablets of Formulation Example 9 are coated in a customary manner with a coating of sucrose, potato starch, talc, tragacanth, and colorant.

### FORMULATION EXAMPLE 11

Injection Vials:

The pH of a solution of 250 g of valdecoxib, 500 g of 6-(5-carboxy-5-methyl-hexyloxy)-2,2-dimethyl-hexanoic acid, calcium salt, and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 12.5 mg of valdecoxib and 25 mg of 6-(5-carboxy-5-methyl-hexyloxy)-2,2-dimethyl-hexanoic acid, calcium salt.

### FORMULATION EXAMPLE 12

Suppositories:

A mixture of 50 g of valdecoxib, 25 g of 6-(5-carboxy-5-methyl-hexyloxy)-2,2-dimethyl-hexanoic acid, calcium salt, 100 g of soya lecithin, and 1400 g of cocoa butter is fused, poured into molds, and allowed to cool. Each suppository contains 50 mg of valdecoxib and 25 mg of 6-(5-carboxy-5-methyl-hexyloxy)-2,2-dimethyl-hexanoic acid, calcium salt.

### FORMULATION EXAMPLE 13

Solution:

A solution is prepared from 0.5 g of valdecoxib, 1 g of 6-(5-carboxy-5-methyl-hexyloxy)-2,2-dimethyl-hexanoic acid, calcium salt, 9.38 g of NaH₂PO₄.12H₂O, 28.48 g of Na₂HPO₄.12H₂O, and 0.1 g benzalkonium chloride in 940 mL of double-distilled water. The pH of the solution is adjusted to pH 6.8 using 2 M hydrochloric acid. The solution is diluted to 1.0 L with double-distilled water, and sterilized by irradiation. A 25 mL volume of the solution contains 12.5 mg of valdecoxib and 25 mg of 6-(5-carboxy-5-methyl-hexyloxy)-2,2-dimethyl-hexanoic acid, calcium salt.

### FORMULATION EXAMPLE 14

Ointment:

100 mg of valdecoxib, 500 mg of 6-(5-carboxy-5-methyl-hexyloxy)-2,2-dimethyl-hexanoic acid, calcium salt is mixed with 99.4 g of petroleum jelly under aseptic conditions. A 5 g portion of the ointment contains 5 mg of valdecoxib and 25 mg of 6-(5-carboxy-5-methyl-hexyloxy)-2,2-dimethyl-hexanoic acid, calcium salt.

### FORMULATION EXAMPLE 15

Capsules:

2 kg of valdecoxib and 20 kg of 6-(5-carboxy-5-methyl-hexyloxy)-2,2-dimethyl-hexanoic acid, calcium salt are filled into hard gelatin capsules in a customary manner such that each capsule contains 25 mg of valdecoxib and 250 mg of 6-(5-carboxy-5-methyl-hexyloxy)-2,2-dimethyl-hexanoic acid, calcium salt.

### FORMULATION EXAMPLE 16

Ampoules:

A solution of 2.5 kg of valdecoxib and 2.5 kg of 6-(5-carboxy-5-methyl-hexyloxy)-2,2-dimethyl-hexanoic acid, calcium salt is dissolved in 60 L of double-distilled water. The solution is sterile filtered, and the filtrate is filled into ampoules. The ampoules are lyophilized under sterile conditions and aseptically sealed. Each ampoule contains 25 mg each of valdecoxib and 6-(5-carboxy-5-methyl-hexyloxy)-2,2-dimethyl-hexanoic acid, calcium salt.

### FORMULATION EXAMPLE 17

Tablet Formulation of
6-(5-carboxy-5-methyl-hexyloxy)-2,2-dimethyl-hexanoic acid,
calcium salt:

| Ingredient | Amount (mg) |
|---|---|
| 6-(5-carboxy-5-methyl-hexyloxy)-2,2-dimethyl- | 25 |
| hexanoic acid, calcium salt | |
| Lactose | 50 |
| Cornstarch (for mix) | 10 |
| Cornstarch (paste) | 10 |
| Magnesium stearate (1%) | 5 |
| Total | 100 |

6-(5-carboxy-5-methyl-hexyloxy)-2,2-dimethyl-hexanoic acid, calcium salt, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet.

Injection Vial Formulation of Valdecoxib:

The pH of a solution of 500 g of valdecoxib and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 25 mg of valdecoxib.

Such tablets containing 6-(5-carboxy-5-methyl-hexyloxy)-2,2-dimethyl-hexanoic acid, calcium salt can be administered to a human from one to four times a day for treatment of the above-listed diseases, and the injection solutions containing valdecoxib can be administered to a human 1 or 2 times per day, wherein the administration by injection is optionally simultaneous with administration of the tablets or at different times, for the treatment of one of the above-listed diseases, including rheumatoid arthritis.

### FORMULATION EXAMPLE 18

Coated Tablets containing 6-(5-carboxy-5-methyl-hexyloxy)-2,2-dimethyl-hexanoic Acid, Calcium Salt:

The tablets of Formulation Example 17 are coated in a customary manner with a coating of sucrose, potato starch, talc, tragacanth, and colorant.

Capsules Containing Valdecoxib:

2 kg of valdecoxib are filled into hard gelatin capsules in a customary manner such that each capsule contains 25 mg of valdecoxib.

Such coated tablets containing 6-(5-carboxy-5-methyl-hexyloxy)-2,2-dimethyl-hexanoic acid, calcium salt can be administered to a human from one to four times a day for treatment of the above-listed diseases, and the capsules containing valdecoxib can be administered to a human 1 or 2 times per day, wherein the administration of the capsules is optionally simultaneous with administration of the tablets or at different times, for the treatment of one of the above-listed diseases.

Other aspects of invention methods, compositions, and combinations are contemplated by the inventors, as illustrated below in the Detailed Description of the Invention.

### BRIEF DESCRIPTION OF THE DRAWINGS

FIG. 1 (“FIG. 1”) is a dose response line graph that shows the change in rat hind paw weight distribution, expressed in grams, on Day 14 in rats with knee joint arthritis, which was induced on Day 0 by injection of a physiologic saline solution of 1 mg of monosodium iodoacetate (“MIA”) through the infrapatellar ligament of the right knee, following oral administration of 10, 30, or 100 mg/kg of 6-(5-carboxy-5-methyl-hexyloxy)-2,2-dimethyl-hexanoic acid, calcium salt. The rat hind paw weight distribution data were measured at Time 0, 2, 4, and 6 hours post compound administration.

FIG. 2 (“FIG. 2”) is a time course line graph that shows the change in rat hind paw weight distribution, expressed in grams, on Days 7, 14, and 28 in rats with knee joint arthritis, which was induced on Day 0 by injection of a physiologic saline solution of 1 mg of monosodium iodoacetate (“MIA”) through the infrapatellar ligament of the right knee, following chronic oral administration b.i.d. of 30 mg/kg of 6-(5-carboxy-5-methyl-hexyloxy)-2,2-dimethyl-hexanoic acid, calcium salt versus vehicle, wherein the compound was administered from 0.5 hour pre-injection of MIA and then approximately every 12 hours through the end of Day 28.

FIG. 3 (“FIG. 3”) is a time course, dose response line graph that shows the change in rat hind paw weight distribution, expressed in grams, on Days 7, 14, and 28 in rats with knee joint arthritis, which was induced on Day 0 by injection of a physiologic saline solution of 1 mg of monosodium iodoacetate (“MIA”) through the infrapatellar ligament of the right knee, following chronic oral administration b.i.d. of 3, 10, or 30 mg/kg of 6-(5-carboxy-5-methyl-hexyloxy)-2,2-dimethyl-hexanoic acid, calcium salt versus vehicle, wherein the compound was administered from 0.5 hour pre-injection of MIA and then approximately every 12 hours through the end of Day 28.

FIG. 4 (“FIG. 4”) is a dose response bar graph that shows effects on (i) cartilage erosion severity, expressed as Grade 0 (no erosion), Grade I (erosion extending into the superficial or middle cartilage layers and further characterized by size small, medium, and large), or Grade II (deep layer erosion; no cartilage remaining in spots, subchondral bone exposed in spots, and further characterized by size small, medium, and large) and (ii) cartilage size, expressed as percent erosion distribution, relative to vehicle-injected control animals, on Day 28 in rats with knee joint arthritis, which was induced on Day 0 by injection of a physiologic saline solution of 1 mg of monosodium iodoacetate (“MIA”) through the infrapatellar ligament of the right knee, following chronic oral administration b.i.d. of 3, 10, or 30 mg/kg of 6-(5-carboxy-5-methyl-hexyloxy)-2,2-dimethyl-hexanoic acid, calcium salt versus vehicle, wherein the compound was administered from 0.5 hour pre-injection of MIA and then approximately every 12 hours through the end of Day 28.

FIG. 5 (“FIG. 5”) is a dose response bar graph that shows effects on total cartilage erosion area, expressed in square millimeters, regardless of cartilage erosion severity grade, relative to vehicle-injected control animals, on Day 28 in rats with knee joint arthritis, which was induced on Day 0 by injection of a physiologic saline solution of 1 mg of monosodium iodoacetate (“MIA”) through the infrapatellar ligament of the right knee, following chronic oral administration b.i.d. of 3, 10, or 30 mg/kg of 6-(5-carboxy-5-methyl-hexyloxy)-2,2-dimethyl-hexanoic acid, calcium salt versus vehicle, wherein the compound was administered from 0.5 hour pre-injection of MIA and then approximately every 12 hours through the end of Day 28.

FIG. 6 (“FIG. 6”) is a time course, dose response line graph that shows the change in rat hind paw weight distribution, expressed in grams, following oral administration at Time-1 hour of 10, 30, or 100 mg/kg of 6-(5-carboxy-5-methyl-hexyloxy)-2,2-dimethyl-hexanoic acid, calcium salt, followed by intra-articular injection through the patellar ligament into the joint space at Time 0 hour of a combination of 100 ng IL-6 and 300 ng of IL-6sR dissolved in 50 μL of PBS, compared to a vehicle treated control group. The rat hind paw weight distribution data were measured at Time 0, 1, 3, and 6 hours post injection or 1, 2, 4, and 7 hours post compound administration, respectively.

FIG. 7 (“FIG. 7”) is a time course, dose response line graph that shows the change in rat hind paw weight distribution, expressed in grams, following oral administration at Time-1 hour of 10 or 30 mg/kg of 6-(5-carboxy-5-methyl-hexyloxy)-2,2-dimethyl-hexanoic acid, calcium salt, followed by intra-articular injection through the patellar ligament into the joint space at Time 0 hour of a combination of 100 ng IL-6 and 300 ng of IL-6sR dissolved in 50 μL of PBS, compared to a vehicle treated control group. The rat hind paw weight distribution data were measured at Time 0, 1, 3, and 6 hours post injection or 1, 2, 4, and 7 hours post compound administration, respectively.

FIG. 8 (“FIG. 8”) is a time course, dose response line graph that shows change in mouse ankle joint and paw swelling, expressed in millimeters (“mm”), following oral administration of vehicle or 30, 100, or 200 mg/kg of 6-(5-carboxy-5-methyl-hexyloxy)-2,2-dimethyl-hexanoic acid, calcium salt, dissolved in oral vehicle beginning on Day-1 and continuing through Day 11 and an intraperitoneal injection of a mixture of monoclonal anti-collagen antibodies on Day 0.

### DETAILED DESCRIPTION OF THE INVENTION

As summarized above, this application relates to methods, compositions, and combinations using or having a substituted dialkyl ether, substituted aryl-alkyl ether, substituted dialkyl thioether, substituted dialkyl ketone, or substituted-alkyl compound, or a pharmaceutically acceptable salt thereof, as an active component for preventing or treating osteoarthritis (“OA”), preventing or inhibiting cartilage damage, preventing or treating rheumatoid arthritis (“RA”), improving joint function, or alleviating pain in a patient in need thereof.

Substituted dialkyl ether, substituted aryl-alkyl ether, substituted dialkyl thioether, substituted dialkyl ketone, or substituted-alkyl compounds useful in an invention method, composition, or combination include any aspect or embodiment of the therapeutic compounds described in U.S. Pat. Nos. 3,773,946; 3,930,024; 4,287,200; 4,689,344; 4,711,896; 5,648,387; 5,750,569; 5,756,544; 5,783,600; 6,410,802; 6,459,003; and 6,506,799; U.S. patent application Ser. Nos. 09/976,867; 09/976,938; 09/976,898; 09/976,899; and 10/205,939; U.S. patent application Publication Numbers U.S. Ser. No. 2002/0077316; U.S. Ser. No. 2003/0018013; U.S. Ser. No. 2003/0022865; U.S. Ser. No. 2003/0065195; and U.S. Ser. No. 2003/0078239; and PCT International Application Publication numbers WO 96/30328; WO 98/30530; WO 00/59855; WO 01/55078; WO 02/30860; WO 02/30863; WO 02/30882; and WO 02/30884, which are each hereby incorporated herein by reference.

Substituted dialkyl ethers of Formula I, and pharmaceutically acceptable salts thereof, including the compound named 6-(5-carboxy-5-methyl-hexyloxy)-2,2-dimethyl-hexanoic acid, calcium salt, are described in U.S. Pat. No. 5,648,387 and its divisionals U.S. Pat. Nos. 5,750,569; 5,756,544; and 5,783,600, and in PCT International Application Publication nos. WO 96/30328; WO 01/55078.

Substituted-alkyl compounds of Formula II, and pharmaceutically acceptable salts thereof, are described in U.S. Pat. No. 3,773,946.

Substituted-alkyl compounds of Formula III, and pharmaceutically acceptable salts thereof, are described in U.S. Pat. No. 3,930,024.

Substituted aryl-alkyl ethers of Formula IV, and pharmaceutically acceptable salts thereof, are described in U.S. Pat. No. 4,287,200.

Substituted dialkyl ether, substituted aryl-alkyl ether, substituted dialkyl thioether, substituted dialkyl ketone, or substituted-alkyl compounds of Formula V, and pharmaceutically acceptable salts thereof, are described in U.S. Pat. No. 4,689,344.

Substituted dialkyl ether, substituted aryl-alkyl ether, substituted dialkyl thioether, substituted dialkyl ketone, or substituted-alkyl compounds of Formula VI, and pharmaceutically acceptable salts thereof, are described in U.S. Pat. No. 4,711,896.

Substituted dialkyl ether, substituted aryl-alkyl ether, substituted dialkyl thioether, substituted dialkyl ketone, or substituted-alkyl compounds of Formula VII, and pharmaceutically acceptable salts thereof, are described in PCT International Patent Application Publication No. WO 98/30530.

Substituted dialkyl ether, substituted aryl-alkyl ether, substituted dialkyl thioether, substituted dialkyl ketone, or substituted-alkyl compounds, and pharmaceutically acceptable salts thereof, are described in U.S. patent application Ser. No. 10/205,939; U.S. Pat. Nos. 6,410,802; 6,459,003; and 6,506,799; in U.S. patent application Publication No. U.S. Ser. No. 2003/0065195; and in PCT International Patent Application Publication No. WO 00/59855.

Substituted dialkyl ether, substituted aryl-alkyl ether, substituted dialkyl thioether, substituted dialkyl ketone, or substituted-alkyl compounds, and pharmaceutically acceptable salts thereof, are described in U.S. patent application Ser. No. 09/976,867; U.S. patent application Publication No. U.S. Ser. No. 2003/0018013; and in PCT International Patent Application Publication No. WO 02/30863.

Substituted dialkyl thioethers are described in U.S. patent application Ser. No. 09/976,898; and 09/976,899; U.S. patent application Publication Nos. U.S. Ser. No. 2002/0077316; and U.S. Ser. No. 2003/0022865; and in PCT International Patent Application Publication Nos. WO 02/30882 and WO 02/30884.

Substituted dialkyl ketones are described in U.S. patent application Ser. No. 09/976,938; U.S. patent application Publication No. U.S. Ser. No. 2003/0078239 and PCT International Patent Application Publication No. WO 02/30860.

It should be appreciated that the compounds utilized in an invention method, composition, or combination are capable of further forming pharmaceutically acceptable salts, including, but not limited to, acid addition and/or base salts. The acid addition salts are formed from basic compounds, whereas the base addition salts are formed from acidic compounds. All of these forms are within the scope of the compounds useful in an invention method, composition, or combination.

Pharmaceutically acceptable acid addition salts of a substituted dialkyl ether, substituted aryl-alkyl ether, substituted dialkyl thioether, substituted dialkyl ketone, or substituted-alkyl compound include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are nontoxic salts of amino acids such as arginate and the like and gluconate, galacturonate (see, for example, Berge S. M. et al., “Pharmaceutical Salts,” J. of Pharma. Sci., 1977;66:1).

An acid addition salt of a substituted dialkyl ether, substituted aryl-alkyl ether, substituted dialkyl thioether, substituted dialkyl ketone, or substituted-alkyl compound is prepared by contacting the free base form of the compound with a sufficient amount of a desired acid to produce a nontoxic salt in the conventional manner. The free base form of the compound may be regenerated by contacting the acid addition salt so formed with a base, and isolating the free base form of the compound in the conventional manner. The free base forms of compounds differ from their respective acid addition salt forms somewhat in certain physical properties such as solubility, crystal structure, hygroscopicity, and the like, but otherwise free base forms of the compounds and their respective acid addition salt forms may be equally utilized in an invention method, composition, or combination.

A pharmaceutically acceptable base addition salt of a substituted dialkyl ether, substituted aryl-alkyl ether, substituted dialkyl thioether, substituted dialkyl ketone, or substituted-alkyl compound may be prepared by contacting the free acid form of the compound with a metal cation such as an alkali or alkaline earth metal cation, or an amine, especially an organic amine. Examples of suitable metal cations include sodium cation (Na⁺), potassium cation (K⁺), magnesium cation (Mg²+), calcium cation (Ca²+), and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977).

A base addition salt of a substituted dialkyl ether, substituted aryl-alkyl ether, substituted dialkyl thioether, substituted dialkyl ketone, or substituted-alkyl compound may be prepared by contacting the free acid form of the compound with a sufficient amount of a desired base to produce the salt in the conventional manner. The free acid form of the compound may be regenerated by contacting the salt form so formed with an acid, and isolating the free acid of the compound in the conventional manner. The free acid forms of the compounds differ from their respective salt forms somewhat in certain physical properties such as solubility, crystal structure, hygroscopicity, and the like, but otherwise the salts may be utilized equally in an invention method, composition, or combination.

The compounds useful in an invention method, composition, or combination may exist in unsolvated forms as well as solvated forms, including hydrated forms. In general, the solvated forms, including hydrated forms, are equivalent to unsolvated forms. An invention method, composition, or combination may utilize any solvated form, including hydrated form, of the compound, as well as mixtures thereof.

The compounds useful in an invention method, composition, or combination may possess one or more chiral centers, and each center may exist in the R or S configuration. An invention method, composition, or combination may utilize any diastereomeric, enantiomeric, or epimeric form of a substituted dialkyl ether, substituted aryl-alkyl ether, substituted dialkyl thioether, substituted dialkyl ketone, or substituted-alkyl compound, or a pharmaceutically acceptable salt thereof, as well as mixtures thereof.

Certain compounds useful in an invention method, composition, or combination may exist as two or more tautomeric forms. Tautomeric forms of the substituted dialkyl ether, substituted aryl-alkyl ether, substituted dialkyl thioether, substituted dialkyl ketone, or substituted-alkyl compounds may interchange, for example, via enolization/de-enolization, 1,2-hydride, 1,3-hydride, or 1,4-hydride shifts, and the like. An invention method, composition, or combination may utilize any tautomeric form of the compound, as well as mixtures thereof.

Some compounds useful in an invention method, composition, or combination have alkenyl groups, which may exist as entgegen or zusammen conformations, in which case all geometric forms thereof, both entgegen and zusammen, cis and trans, and mixtures thereof, may be utilized in an invention method, composition, or combination.

Some compounds useful in an invention method, composition, or combination have cycloalkyl groups, which may be substituted at more than one carbon atom, in which case all geometric forms thereof, both cis and trans, and mixtures thereof, may be used in an invention method, composition, or combination.

Some compounds useful in an invention method, composition, or combination may exist as amorphous or crystalline solids, in which case all physical forms thereof, including clathrates thereof and mixtures thereof, may be used in an invention method, composition, or combination.

Invention methods, compositions, or combinations also utilize isotopically-labelled compounds useful in an invention method, composition, or combination, which are identical to those recited above, but for the fact that one or more atoms are replaced by an atom having an atomic mass or mass number different from the atomic mass or mass number usually found in nature. Examples of isotopes that can be incorporated into compounds utilized in an invention method, composition, or combination include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹p, ³²P, ³⁵S, ¹⁸F and ³⁶Cl, respectively. Compounds and pharmaceutically acceptable salts of said compounds that contain the aforementioned isotopes and/or other isotopes of other atoms may be utilized in an invention method, composition, or combination. Certain isotopically labelled compounds utilized in an invention method, composition, or combination, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are known for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be utilized in some circumstances. Isotopically labelled compounds of those described above in an invention method, composition, or combination can generally be prepared by carrying out the procedures incorporated by reference above and below, or procedures disclosed in the Schemes and/or in the Examples and Preparations, if any, disclosed herein, by substituting a readily available isotopically labelled reagent for a non-isotopically labelled reagent.

As mentioned above, an aspect of the invention is a method that prevents or inhibits cartilage tissue damage. The ability of the compounds useful in an invention method, composition, or combination to prevent or inhibit cartilage damage may be evidenced by the beneficial effects of the active compounds on synovial changes and lesions on tibial plateaus, and reduced damage to femoral condyles and tibial plateaus. Additional evidence may include the active compounds' beneficial effects on proteoglycan content of a joint.

The compounds useful in an invention method, composition, or combination can also be used in combination with existing therapeutic agents for the treatment of osteoarthritis or rheumatoid arthritis, or the alleviation of pain. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib (tradename CELEBREX® by G. D. Searle & Co., Skokie, Ill.), valdecoxib (tradename BEXTRA® by Pharmacia & Upjohn Company, North Peapack, N.J.), etoricoxib (tradename ARCOXIA® by Merck & Co., Inc., Whitehouse Station, N.J.), parecoxib, and rofecoxib (tradename VIOXX® by Merck & Co., Inc., Whitehouse Station, N.J.) analgesics and intraarticular therapies such as corticosteroids and hyaluronic acids such as hyalgan and synvisc.

Another aspect of the invention relates to a method of, and a pharmaceutical composition for, treating inflammatory processes and diseases comprising administering an active compound, composition, or combination to a mammal, including a human, cat, livestock or dog, wherein said inflammatory processes and diseases are defined as above and said compound is used in combination with one or more other therapeutically active agents under the following conditions:

A.) where a joint has become seriously inflamed as well as infected at the same time by bacteria, fungi, protozoa and/or virus, said inhibitory combination is administered in combination with one or more antibiotic, antifungal, antiprotozoal and/or antiviral therapeutic agents;

B.) where a multi-fold treatment of pain and inflammation is desired, said inhibitory combination is administered in combination with inhibitors of other mediators of inflammation, comprising one or more members independently selected from the group consisting essentially of:

(1) NSAIDs;

(2) H₁-receptor antagonists;

(3) kinin-B₁- and B₂-receptor antagonists;

(4) prostaglandin inhibitors selected from the group consisting of PGD-, PGF- PGI₂- and PGE-receptor antagonists;

(5) thromboxane A₂ (TXA₂-) inhibitors;

(6) 5-, 12- and 15-lipoxygenase inhibitors;

(7) leukotriene LTC₄-, LTD₄/LTE₄- and LTB₄-inhibitors;

(8) PAF-receptor antagonists;

(9) gold in the form of an aurothio group together with one or more hydrophilic groups;

(10) immunosuppressive agents selected from the group consisting of cyclosporine, azathioprine and methotrexate;

(11) anti-inflammatory glucocorticoids;

(12) penicillamine;

(13) hydroxychloroquine;

(14) anti-gout agents including colchicine; xanthine oxidase inhibitors including allopurinol; and uricosuric agents selected from probenecid, sulfinpyrazone and benzbromarone;

C. where older mammals are being treated for disease conditions, syndromes and symptoms found in geriatric mammals, said inhibitory combination is administered in combination with one or more members independently selected from the group consisting essentially of:

(1) cognitive therapeutics to counteract memory loss and impairment;

(2) anti-hypertensives and other cardiovascular drugs intended to offset the consequences of atherosclerosis, hypertension, myocardial ischemia, angina, congestive heart failure and myocardial infarction, selected from the group consisting of:

a. diuretics;

b. vasodilators;

c. β-adrenergic receptor antagonists;

d. angiotensin-II converting enzyme inhibitors (ACE-inhibitors), alone or optionally together with neutral endopeptidase inhibitors;

e. angiotensin II receptor antagonists;

f. renin inhibitors;

g. calcium channel blockers;

h. sympatholytic agents;

i. α₂-adrenergic agonists;

j. α-adrenergic receptor antagonists; and

k. HMG-CoA-reductase inhibitors (anti-hypercholesterolemics);

(3) antineoplastic agents selected from:

a. antimitotic drugs selected from:

i. vinca alkaloids selected from:

[1] vinblastine and

[2] vincristine;

(4) growth hormone secretagogues;

(5) strong analgesics;

(6) local and systemic anesthetics; and

(7) H₂-receptor antagonists, proton pump inhibitors and other gastroprotective agents.

The compounds useful in an invention method, composition, or combination may be administered in combination with inhibitors of other mediators of inflammation, comprising one or more members selected from the group consisting essentially of the classes of such inhibitors and examples thereof which include, matrix metalloproteinase inhibitors, aggrecanase inhibitors, TACE inhibitors, leucotriene receptor antagonists, IL-1 processing and release inhibitors, ILra, H₁-receptor antagonists; kinin-B₁- and B₂-receptor antagonists; prostaglandin inhibitors such as PGD-, PGF- PGI₂-and PGE-receptor antagonists; thromboxane A₂ (TXA2-) inhibitors; 5- and 12-lipoxygenase inhibitors; leukotriene LTC₄-, LTD₄/LTE₄- and LTB₄-inhibitors; PAF-receptor antagonists; MEK inhibitors; IKK inhibitors; MKK inhibitors; gold in the form of an aurothio group together with various hydrophilic groups; immunosuppressive agents, e.g., cyclosporine, azathioprine and methotrexate; anti-inflammatory glucocorticoids; penicillamine; hydroxychloroquine; anti-gout agents, e.g., colchicine, xanthine oxidase inhibitors, e.g., allopurinol and uricosuric agents, e.g., probenecid, sulfinpyrazone and benzbromarone.

The compounds useful in an invention method, composition, or combination may also be used in combination with anticancer agents such as endostatin and angiostatin or cytotoxic drugs such as adriamycin, daunomycin, cis-platinum, etoposide, taxol, taxotere and alkaloids, such as vincristine and antimetabolites such as methotrexate.

The compounds useful in an invention method, composition, or combination may also be used in combination with anti-hypertensives and other cardiovascular drugs intended to offset the consequences of atherosclerosis, including hypertension, myocardial ischemia including angina, congestive heart failure and myocardial infarction, selected from vasodilators such as hydralazine, β-adrenergic receptor antagonists such as propranolol, calcium channel blockers such as nifedipine, α₂-adrenergic agonists such as clonidine, α-adrenergic receptor antagonists such as prazosin and HMG-CoA-reductase inhibitors (anti-hypercholesterolermics) such as lovastatin or atorvastatin.

The compounds useful in an invention method, composition, or combination may also be administered in combination with one or more antibiotic, antifungal, antiprotozoal, antiviral or similar therapeutic agents.

The compounds useful in an invention method, composition, or combination may also be used in combination with CNS agents such as antidepressants (such as sertraline), anti-Parkinsonian drugs (such as L-dopa, requip, mirapex, MAOB inhibitors such as selegine and rasagiline, comP inhibitors such as Tasmar, A-2 inhibitors, dopamine reuptake inhibitors, NMDA antagonists, nicotine agonists, dopamine agonists and inhibitors of neuronal nitric oxide synthase) and anti-Alzheimer's drugs such as donepezil, tacrine, COX-2 inhibitors, propentofylline or metryfonate.

The compounds useful in an invention method, composition, or combination may also be used in combination with osteoporosis agents such as roloxifene, lasofoxifene, droloxifene or fosomax and immunosuppressant agents such as FK-506 and rapamycin.

Other mammalian diseases and disorders which are treatable by administration of an invention combination alone, or contained in a pharmaceutical composition as defined below, include: fever (including rheumatic fever and fever associated with influenza and other viral infections), common cold, dysmenorrhea, menstrual cramps, inflammatory bowel disease, Crohn's disease, emphysema, acute respiratory distress syndrome, asthma, bronchitis, chronic obstructive pulmonary disease, Alzheimer's disease, organ transplant toxicity, cachexia, allergic reactions, allergic contact hypersensitivity, cancer (such as solid tumor cancer including colon cancer, breast cancer, lung cancer and prostrate cancer; hematopoietic malignancies including leukemias and lymphomas; Hodgkin's disease; aplastic anemia, skin cancer and familiar adenomatous polyposis), tissue ulceration, peptic ulcers, gastritis, regional enteritis, ulcerative colitis, diverticulitis, recurrent gastrointestinal lesion, gastrointestinal bleeding, coagulation, anemia, synovitis, gout, ankylosing spondylitis, restenosis, periodontal disease, epidermolysis bullosa, osteoporosis, loosening of artificial joint implants, atherosclerosis (including atherosclerotic plaque rupture), aortic aneurysm (including abdominal aortic aneurysm and brain aortic aneurysm), periarteritis nodosa, congestive heart failure, myocardial infarction, stroke, cerebral ischemia, head trauma, spinal cord injury, neuralgia, neuro-degenerative disorders (acute and chronic), autoimmune disorders, Huntington's disease, Parkinson's disease, migraine, depression, peripheral neuropathy, pain (including low back and neck pain, headache and toothache), gingivitis, cerebral amyloid angiopathy, nootropic or cognition enhancement, amyotrophic lateral sclerosis, multiple sclerosis, ocular angiogenesis, corneal injury, macular degeneration, conjunctivitis, abnormal wound healing, muscle or joint sprains or strains, tendonitis, skin disorders (such as psoriasis, eczema, scleroderma and dermatitis), myasthenia gravis, polymyositis, myositis, bursitis, burns, diabetes (including types I and II diabetes, diabetic retinopathy, neuropathy and nephropathy), tumor invasion, tumor growth, tumor metastasis, corneal scarring, scleritis, immunodeficiency diseases (such as AIDS in humans and FLV, FIV in cats), sepsis, premature labor, hypoprothrombinemia, hemophilia, thyroiditis, sarcoidosis, Behcet's syndrome, hypersensitivity, kidney disease, Rickettsial infections (such as Lyme disease, Erlichiosis), Protozoan diseases (such as malaria, giardia, coccidia), reproductive disorders (for example in livestock), epilepsy, convulsions, and septic shock.

The substituted dialkyl ether, substituted aryl-alkyl ether, substituted dialkyl thioether, substituted dialkyl ketone, or substituted-alkyl compounds disclosed herein are useful in human and veterinary medicines for preventing and treating osteoarthritis, preventing and treating rheumatoid arthritis, improving joint function, alleviating pain, including, but not limited to, OA pain, RA pain, joint pain, inflammatory pain, acute pain, chronic pain, bone cancer pain, pain mediated by IL-6, IL-6sR, or a combination of IL-6 and IL-6sR, pain mediated by endothelin-1, static allodynia, dynamic allodynia, mechanical pain, headache pain, and the like, and preventing and inhibiting cartilage damage in a mammal, and for treating any other disease or disorder wherein cartilage damage is a symptom or is involved in the underlying pathology of the condition being treated.

The terms and phrases used herein are as defined below or as they otherwise occur in the specification.

The phrase “substituted dialkyl ether, substituted aryl-alkyl ether, substituted dialkyl thioether, substituted dialkyl ketone, or substituted-alkyl compound” is synonymous with the phrase “active compound,” which refers to a substituted dialkyl ether, substituted aryl-alkyl ether, substituted dialkyl thioether, substituted dialkyl ketone, or substituted-alkyl compound that is useful in an invention method, composition, or combination, or a pharmaceutically acceptable salt thereof, or any other form thereof that is described herein. A substituted dialkyl ether, substituted aryl-alkyl ether, substituted dialkyl thioether, substituted dialkyl ketone, or substituted-alkyl compound is any compound, including a cyclic ether, as described above, including oxo-substituted ether compounds wherein the ether oxygen atom comprises a functional group of the formula (A):

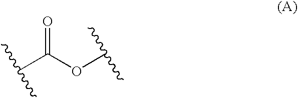

and oxo-substituted thioether compounds, which are substituted dialkyl sulfoxides or substituted dialkyl sulfones.

It should be appreciated that any compound described herein as an active compound may be used in any invention method, composition, or combination.

It should be appreciated that the substituted dialkyl ether named 6-(5-carboxy-5-methyl-hexyloxy)-2,2-dimethyl-hexanoic acid may be represented by the structure drawn below:

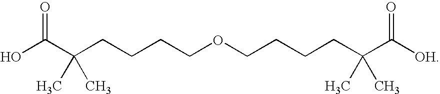

The substituted dialkyl ether named 6-(5-carboxy-5-methyl-hexyloxy)-2,2-dimethyl-hexanoic acid, calcium salt, may be represented by the structure drawn below:

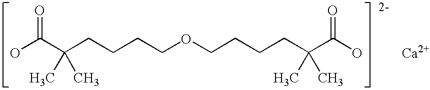

The substituted dialkyl ether named 6-(5-carboxy-5-methyl-hexyloxy)-2,2-dimethyl-hexanoic acid, calcium salt is known by other names, including “6-(5-carboxy-5-methyl-hexyloxy)-2,2-dimethyl-hexanoic acid, monocalcium salt,” “6-(5-carboxy-5-methyl-hexyloxy)-2,2-dimethyl-hexanoic acid, mono-calcium salt, “6,6′-oxybis(2,2-dimethylhexanoic acid),” “CI-1027 ” and gemcabene calcium. CI-1027 has been in clinical development for the treatment of dyslipidemia. CI-1027 is a substituted dialkyl ether as defined herein that is a monocalcium salt.

It should be appreciated that the substituted dialkyl ether named 6-(5-carboxy-5-methyl-hexyloxy)-2,2-dimethyl-hexanoic acid, calcium salt may exist in a number of different physical forms, including Crystal Form 1 and Crystal Form 2. Crystal Form 1 and Crystal Form 2 of the substituted dialkyl ether named 6-(5-carboxy-5-methyl-hexyloxy)-2,2-dimethyl-hexanoic acid, calcium salt have disclosed in PCT International Patent Application Publication No. WO 01/55078. The use of each of these crystal forms is within the scope of this invention method.

Crystal Form 1 has an x-ray powder diffraction pattern substantially comprising:

| # | 2-Theta | d(A) | Peak | P % | Area | Area % | FWHM |
|---|---|---|---|---|---|---|---|
| 1 | 6.760 | 13.0648 | 5106 | 100.0 | 1497 | 100.0 | 0.234 |
| 2 | 8.183 | 10.7953 | 1743 | 34.1 | 435 | 29.1 | 0.200 |
| 3 | 8.560 | 10.3207 | 1866 | 36.5 | 543 | 36.3 | 0.233 |
| 4 | 9.239 | 9.5638 | 234 | 4.6 | 29 | 1.9 | 0.096 |
| 5 | 9.760 | 9.0546 | 972 | 19.0 | 220 | 14.7 | 0.181 |
| 6 | 10.569 | 8.3634 | 156 | 3.1 | 12 | 0.8 | 0.061 |
| 7 | 11.141 | 7.9353 | 178 | 3.5 | 29 | 1.9 | 0.130 |
| 8 | 13.760 | 6.4304 | 266 | 5.2 | 46 | 3.1 | 0.138 |
| 9 | 15.599 | 5.6761 | 338 | 6.6 | 63 | 4.2 | 0.148 |
| 10 | 16.740 | 5.2917 | 433 | 8.5 | 64 | 4.3 | 0.118 |
| 11 | 17.420 | 5.0866 | 1890 | 37.0 | 689 | 46.0 | 0.291 |
| 12 | 20.639 | 4.3000 | 523 | 10.2 | 128 | 8.5 | 0.196 |
| 13 | 21.391 | 4.1505 | 188 | 3.7 | 20 | 1.3 | 0.085 |
| 14 | 22.139 | 4.0119 | 445 | 8.7 | 74 | 4.9 | 0.132 |
| 15 | 31.559 | 2.8326 | 270 | 5.3 | 24 | 1.6 | 0.070 |

Crystal Form 2 has an x-ray powder diffraction pattern substantially comprising:

| # | 2-Theta | d(A) | Peak | P % | Area | Area % | FWHM |
|---|---|---|---|---|---|---|---|
| 1 | 7.259 | 12.1686 | 9283 | 100.0 | 2482 | 100.0 | 0.214 |
| 2 | 8.739 | 10.1100 | 4191 | 45.1 | 603 | 24.3 | 0.115 |
| 3 | 9.386 | 8.9628 | 967 | 10.4 | 161 | 6.5 | 0.133 |
| 4 | 11.659 | 7.5838 | 430 | 4.6 | 49 | 1.9 | 0.089 |
| 5 | 13.955 | 6.3408 | 305 | 3.3 | 58 | 2.3 | 0.151 |
| 6 | 14.220 | 6.2233 | 326 | 3.5 | 73 | 2.9 | 0.178 |
| 7 | 15.387 | 5.7537 | 278 | 3.0 | 19 | 0.7 | 0.053 |
| 8 | 16.461 | 5.3806 | 986 | 10.6 | 187 | 7.5 | 0.152 |
| 9 | 17.361 | 5.1039 | 1490 | 16.1 | 348 | 14.0 | 0.187 |
| 10 | 18.063 | 4.9069 | 1284 | 13.8 | 323 | 13.0 | 0.201 |
| 11 | 19.302 | 4.5947 | 871 | 9.4 | 166 | 6.7 | 0.152 |
| 12 | 19.862 | 4.4664 | 686 | 7.4 | 142 | 5.7 | 0.166 |
| 13 | 20.200 | 4.3923 | 457 | 4.9 | 103 | 4.1 | 0.179 |
| 14 | 21.178 | 4.1918 | 656 | 7.1 | 97 | 3.9 | 0.117 |
| 15 | 21.641 | 4.1031 | 167 | 1.8 | 6 | 0.2 | 0.029 |
| 16 | 22.300 | 3.9833 | 794 | 8.6 | 192 | 7.7 | 0.193 |
| 17 | 23.218 | 3.8278 | 247 | 2.7 | 23 | 0.9 | 0.071 |
| 18 | 24.100 | 3.6897 | 183 | 2.0 | 34 | 1.3 | 0.145 |
| 19 | 25.481 | 3.4928 | 487 | 5.2 | 141 | 5.7 | 0.231 |
| 20 | 28.800 | 3.0974 | 134 | 1.4 | 14 | 0.6 | 0.083 |
| 21 | 29.297 | 3.0459 | 259 | 2.8 | 28 | 1.1 | 0.084 |
| 22 | 30.700 | 2.9099 | 287 | 3.1 | 20 | 0.8 | 0.055 |

It should be appreciated that the substituted dialkyl ether named 6-(5-carboxy-5-methyl-hexyloxy)-2,2-dimethyl-hexanoic acid, calcium salt, may further exist as a hydrate, known by the name 6-(5-carboxy-5-methyl-hexyloxy)-2,2-dimethyl-hexanoic acid, monocalcium salt hydrate in PCT International Patent Application Publication No. WO 01/55078. The use of this or another hydrate form is within the scope of this invention method.

It should be appreciated that the substituted dialkyl ether named 6-(5-carboxy-5-methyl-hexyloxy)-2,2-dimethyl-hexanoic acid, calcium salt, may further exist as a C₁-C₁₂ alcohol solvate, including an ethyl alcohol, methanol, 1-propyl alcohol, 2-propyl alcohol, or 1-butyl alcohol solvate, known by the names 6-(5-carboxy-5-methyl-hexyloxy)-2,2-dimethyl-hexanoic acid, mono-calcium salt ethyl alcohol solvate, 6-(5-carboxy-5-methyl-hexyloxy)-2,2-dimethyl-hexanoic acid, mono-calcium salt methanol solvate, 6-(5-carboxy-5-methyl-hexyloxy)-2,2-dimethyl-hexanoic acid, monocalcium salt 1-propyl alcohol solvate, 6-(5-carboxy-5-methyl-hexyloxy)-2,2-dimethyl-hexanoic acid, monocalcium salt 2-propyl alcohol solvate, 6-(5-carboxy-5-methyl-hexyloxy)-2,2-dimethyl-hexanoic acid, monocalcium salt 1-butyl alcohol solvate, respectively, in PCT International Patent Application Publication No. WO 01/55078. The use of these and other alcohol solvate forms is within the scope of this invention method.

The phrase “therapeutic biological agent” includes CP-870, etanercept (a tumor necrosis factor alpha (“TNF-alpha”) receptor immunoglobulin molecule; trade names ENBREL® and ENBREL ENTANERCEPT® by Immunex Corporation, Seattle, Wash.), infliximab (an anti-TNF-alpha chimeric IgG 1K monoclonal antibody; tradename REMICADE® by Centocor, Inc., Malvern, Pa.), methotrexate (tradename RHEUMATREX® by American Cyanamid Company, Wayne, N.J.), and adalimumab (a human monoclonal anti-TNF-alpha antibody; tradename HUMIRA® by Abbott Laboratories, Abbott Park, Ill.).

The term “Etanercept” means a dimeric fusion protein consisting of the extracellular ligand-binding portion of the human 75 kilodalton (“p75”) tumor necrosis factor receptor (“TNFR”) linked to the Fc portion of human IgG1. The Fc component of etanercept contains the C_{H}2 domain, the C_{H}3 domain and hinge region, but not the C_{H}1 domain of IgG1. Etanercept is produced by recombinant DNA technology in a Chinese hamster ovary (“CHO”) mammalian cell expression system. It consists of 934 amino acids and has an apparent molecular weight of approximately 150 kilodaltons. Etanercept is an inhibitor of tumor necrosis factor alpha (“TNFalpha”).

Etanercept is marketed in the United States under the tradename ENBREL® and ENBREL ENTANERCEPT® for the treatment of rheumatoid arthritis and psoriatic arthritis. ENBREL® and ENBREL ENTANERCEPT® are registered by Immunex Corporation, Seattle, Wash. ENBREL® is supplied as a sterile, white, preservative-free, lyophilized powder for parenteral administration after reconstitution with 1 mL of the supplied Sterile Bacteriostatic Water for Injection, USP (containing 0.9% benzyl alcohol). Following reconstitution, the solution of ENBREL® is clear and colorless, with a pH of 7.4±0.3. Each single-use vial of ENBREL(D contains 25 mg etanercept, 40 mg mannitol, 10 mg sucrose, and 1.2 mg tromethamine.

The term “infliximab” includes the product marketed in the United States under the tradename REMICADE® for the treatment of rheumatoid arthritis. REMICADE® is registered by Centocor, Inc., Malvern, Pa.

The term “methotrexate” includes a compound named N-[4-[[(2,4-diamino-6-pteridinyl)methyl]methylamino]benzoyl]-L-glutamic acid, or a pharmaceutically acceptable salt thereof. Methotrexate is used in the treatment of certain neoplastic diseases, severe psoriasis, and adult rheumatoid arthritis. For example, Methotrexate Sodium Tablets for oral administration are available in a packaging system designated as the RHEUMATREX® Methotrexate Sodium Dose Pack for therapy with a weekly dosing schedule of 5 mg, 7.5 mg, 10 mg, 12.5 mg, and 15 mg of methotrexate and the following pharmaceutically acceptable excipients, diluents, or carriers: lactose, magnesium stearate, and pregelatinized starch. RHEUMATREX® is registered by American Cyanamid Company, Wayne, N.J. The tablets may also contain cornstarch. Methotrexate is also administered by injection intramuscularly, intravenously, intra-arterially, or intrathecally.

It should be appreciated that COX-2 is also known as prostaglandin synthase-2, prostaglandin PGH₂ synthase, and prostaglandin-H₂ synthase-2.

A selective inhibitor of COX-2 means compounds that inhibit COX-2 selectively versus COX-1 such that a ratio of IC₅₀ for a compound with COX-1 divided by a ratio of IC₅₀ for the compound with COX-2 is greater than, or equal to, 5, where the ratios are determined in one or more assays. All that is required to determine whether a compound is a selective COX-2 inhibitor is to assay a compound in one of a number of well know assays in the art.

It should be appreciated that two forms of cyclooxygenase (“COX”) are now known, namely a constitutive isoform usually named cyclooxygenase-1(“COX-1”) and an inducible isoform usually named cyclooxygenase-2 (“COX-2”), the latter of which expression is upregulated at sites of inflammation. COX-1 appears to play a physiological role and to be responsible for gastrointestinal and renal protection. On the other hand, COX-2 appears to play a pathological role and is believed to be the predominant isoform present in inflammation conditions. The therapeutic use of conventional COX inhibitors, which are typically nonselective inhibitors of both COX-1 and COX-2, is limited due to drug associated side effects, including life threatening ulceration and renal toxicity. Compounds that selectively inhibit COX-2 would exert anti-inflammatory effects without the adverse side effects associated with COX-1 inhibition.

For the purposes of this invention, a selective inhibitor of COX-2 includes a compound, or a pharmaceutically acceptable salt thereof, selected from:

LAS-34475;

UR-8880;

ABT-963;

Valdecoxib;

BMS-347070;

Celecoxib;

Tilacoxib;

The compound of formula (B)

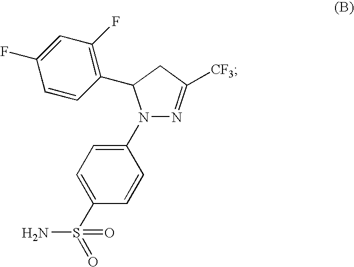

CS-502 [Chemical Abstracts Service Registry Number (“CAS Reg. No.”) 176429-82-6];

(6aR, 10aR)-3-(1,1-dimethylheptyl)-6a,7,10,10a-tetrahydro-1-hydroxy-6,6-dimethyl-6H-dibenzo[b,d]pyran-9-carboxylic acid (“CT-3”);

CV-247;

2(5H)-Furanone, 5,5-dimethyl-3-(1-methylethoxy)-4-[4-(methylsulfonyl)phenyl]-(“DFP”);

Etoricoxib (tradename ARCOXIA® by MERCK & CO., Inc., Whitehouse Station, N.J.);

GW-406381;

Tiracoxib;

Meloxicam;

Nimesulide;

2-(Acetyloxy)benzoic acid, 3-[(nitrooxy)methyl]phenyl ester (“NCX-4016”);

Parecoxib (trade name application pending for DYNASTAT® by G. D. Searle & Co., Skokie, Ill.);

P54 (CAS Reg. No. 130996-28-0);

Rofecoxib (tradename VIOXX® by MERCK & CO., Inc., Whitehouse Station, N.J.);

RevIMiD;

2,6-Bis(1,1-dimethylethyl)-4-[(E)-(2-ethyl-1,1-dioxo-5-isothiazolidinylidene)methyl]phenol (“S-2474”);

5(R)-Thio-6-sulfonamide-3(2H)-benzofuranone (“SVT-2016”); and

N-[3-(Formylamino)-4-oxo-6-phenoxy-4H-1-benzopyran-7-yl]-methanesulfonamide (“T-614”); or

a pharmaceutically acceptable salt thereof.

The term “celecoxib” means the compound named 4-(5-(4-methylphenyl)-3-(trifluoromethyl)-1H-pyrazol-1-yl)-benzenesulfonamide. Celecoxib is a selective cyclooxygenase-2 (“COX-2”) inhibitor currently approved by the FDA for the treatment of osteoarthritis, rheumatoid arthritis, and Polyposis-familial adenomatus. Celecoxib is marketed under the tradename “CELEBREX®”. Celecoxib is currently in clinical trials for the treatment of bladder cancer, chemopreventative-lung cancer, and post-operative pain, and is registered for the treatment of dysmenorrhea. Celecoxib has the structure drawn below:

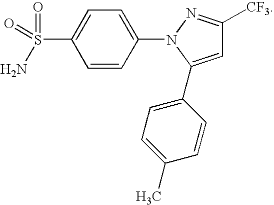

The term “valdecoxib” means the compound named 4-(5-methyl-3-phenyl-4-isoxazolyl)-benzenesulfonamide, which is described in U.S. Pat. Nos. 5,633,272; 5,859,257; and 5,985,902, which are hereby incorporated by reference herein. Valdecoxib has been approved by the FDA for treating osteoarthritis, rheumatoid arthritis, dysmenorrhea, and general pain, and is marketed under the tradename “BEXTRA®”. Valdecoxib is in clinical trials for the treatment of migraine. Valdecoxib has the structure drawn below:

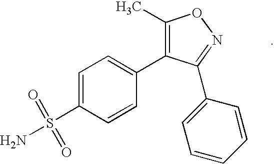

It should be appreciated that the terms “uses”, “utilizes”, and “employs”, and their derivatives thereof, are used interchangeably when describing an aspect of an invention method, composition, or combination.

The terms “including,” “having,” and “containing” are open ended unless otherwise indicated.

The phrase “admixed” or “in admixture” means the ingredients so mixed comprise either a heterogeneous or homogeneous mixture. In some circumstances a homogeneous mixture is preferred. In other circumstances, a heterogeneous mixture is preferred.

The term “ED₄₀” means the dose of a drug, including an active compound, or a pharmaceutically acceptable salt thereof, that is sufficient to inhibit cartilage damage or treat a disease or disorder listed above, in at least 40% of the patients being treated.

The term “drug”, which is synonymous with the phrases “therapeutic agent”, “active component”, “active compound”, and “active ingredient”, includes a nontoxic therapeutic agent such as an active compound, celecoxib, or a pharmaceutically acceptable salt thereof, valdecoxib, or a pharmaceutically acceptable salt thereof, and may further include one or two of the other therapeutic agents described above.

The term “nontoxic” means the efficacious dose is 10 times or greater than the dose at which a toxic effect is observed in 10% or more of a patient population.

The term “patient” means a mammal, and the two terms are used interchangeably herein.

For the purposes of this invention, the term “mammal” includes humans, companion animals such as cats and dogs, livestock animals such as horses, cows, pigs, goats, and sheep, and laboratory animals such as guinea pigs, rabbits, rats, mice, hamsters, and monkeys, and transgenic variants thereof. A human patient is preferred. Also preferred are companion animals, particularly dogs, cats, and horses. Also preferred are laboratory animals, particularly rabbits, rats, mice, and monkeys, and transgenic variants thereof.

The phrase “companion animals” includes dogs, cats, rabbits, hamsters, monkeys, horses, and other household or barnyard pets.

The phrase “livestock animals” as used herein refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus, or for searching and sentinel duty, e.g., a canine animal including domestic dogs and other members of the genus Canis; and domesticated quadrupeds being raised primarily for recreational purposes, e.g., members of Equus and Canis, as well as a feline animal including domestic cats and other members of the family Felidae, genus Felis.

For the purposes of this invention, the term “arthritis” includes, but is not limited to, osteoarthritis, rheumatoid arthritis, degenerative joint disease, spondyloarthropathies, ankylosing spondylitis, infectious arthritis, reactive arthritis, gouty arthritis, systemic lupus erythematosus, juvenile arthritis, and psoriatic arthritis. The substituted dialkyl ether, substituted aryl-alkyl ether, substituted dialkyl thioether, substituted dialkyl ketone, or substituted-alkyl compounds useful in an invention method, composition, or combination may also be useful for treating degenerative joint disease, spondyloarthropathies, gouty arthritis, systemic lupus erythematosus, juvenile arthritis, and psoriatic arthritis.

The phrase “cartilage damage” means a disorder of articular cartilage and subchondral bone characterized by hypertrophy of tissues in and around an involved joint, which may or may not be accompanied by deterioration of articular cartilage surface. As used herein, the phrase cartilage damage relates to damage to joint cartilage.

It should be appreciated that cartilage is a multicellular tissue found at joint linings and in other parts of the body, including the nose, for example. Cartilage tissue provides frictionless surfaces for joint movement, and structure and support for soft tissue features of the body such as the nostrils of the nose. When cartilage tissue is damaged by disease or trauma, breakdown products are formed and the physiological function of the tissue is impaired. There principally are three types of cartilage in a body, including articular cartilage.

The phrase “inhibiting cartilage damage” means the therapeutic effect of a compound, or a combination as described above, that eliminates, alleviates, inhibits or prevents the onset of, inhibits the progress of, prevents further progress of, or reverses progression of, in part or in whole, any one or more pathological hallmarks or symptoms of cartilage damage observed for any of the diseases and disorders which have cartilage damage as a component of the disease or disorder pathology. A patient at risk for developing cartilage damage may be prophylactically treated just as a patient having cartilage damage may be medically treated.

It should be appreciated that a pathological hallmark of a disease or disorder relates to a structural change in a body that is a direct or indirect result of the body being afflicted with the disease or disorder. Such structural changes may be identified by clinical observation, examination of biopsied tissue, pathological examination or by imaging techniques such as X-ray or magnetic resonance imaging, of the affected structure. Illustrative examples of a pathological hallmark include histopathological damage to cartilage, thickening or thinning of bone, hypertrophy of muscle, fibrosis, a tear in a ligament or tendon, and the like.

The term “osteoarthritis” includes diseases of the joint principally characterized by the pathological hallmark of joint cartilage damage, and optionally the symptom of joint pain. Osteoarthritis patients typically do not suffer from inflammation of the joint, although they may experience transient inflammatory flares from time to time.

The term “rheumatoid arthritis” includes rheumatic diseases of the joint principally characterized by the symptom of joint inflammation, and optionally joint pain. Rheumatoid arthritis patients may eventually also experience damage to joint cartilage.

The term “treating” means administration of one or more of the compounds or combinations according to the invention method as defined above that eliminates, alleviates, inhibits or prevents the onset of, inhibits the progress of, prevents further progress of, or reverses progression of, in part or in whole, any one or more of the pathological hallmarks or symptoms of any one of the diseases and disorders being treated, including, but not limited to, the pathological hallmark of cartilage damage and the symptoms of pain and inflammation. A patient at risk for developing a disease or disorder may be prophylactically treated just as a patient having the disease or disorder may be medically treated.

The term “preventing” means prophylactic administration, according to the invention method as defined above, of one or more of the active compounds or combinations of the invention, to an asymptomatic patient at risk for the disease or disorder being prevented to inhibit the onset of an associated pathological hallmark or symptom, including, but not limited to, the pathological hallmark of cartilage damage and the symptoms of pain and inflammation. Further, once onset of a pathological hallmark or symptom has begun, preventing means to prevent further progression or reverse progression, in part or in whole, of the pathological hallmark or symptom.

As mentioned above, an active compound may be administered prophylactically to prevent or inhibit the onset of osteoarthritis, rheumatoid arthritis, loss of joint function, cartilage damage, or any pain in an asymptomatic patient (mammal). It should be appreciated that an asymptomatic patient at risk for the disease or disorder being prevented may be identified by analysis of genetic risk factors (inherited or spontaneous mutation diseases and disorders), family medical history, occupation, participation in athletic activities, general medical screening, and the like.

The term “improving” means administration of one or more of the compounds or combinations according to the invention method as defined above that eliminates or prevents the loss, inhibits further loss, or improves, in part or in whole, of any one or more of the clinical measures of a function in a patient suffering from any one of the diseases and disorders being improved, including, but not limited rheumatoid arthritis and osteoarthritis.

The phrase “joint function” relates to any one or more of the clinical assessments of joint function, including stiffness, range of movement, flexibility, and movement-related symptoms (e.g., altered gait, pain, warmth, or inflammation), in a patient suffering from any one of the diseases and disorders being improved, including, but not limited the diseases of rheumatoid arthritis and osteoarthritis. The Western Ontario and McMaster Universities Osteoarthritis Index (“WOMAC”) may be used by a clinician to assess joint function.

The phrase “pain alleviating” means the effect of one or more of the compounds or combinations according to the invention method as defined above that eliminates, or inhibits or prevents onset of, suppresses, reduces, prevents, or otherwise inhibits, pain in a patient, including, but not limited to, the suppression, reduction, prevention, inhibition or elimination of pain symptoms due to cartilage damage, acute pain, chronic pain, mechanical pain, static allodynia, dynamic allodynia, bone cancer pain, headache, osteoarthritic pain, inflammatory pain, and pain associated with autoimmune disorders or fibromyalgia.

The phrase “joint pain” means any pain in a joint.

The phrase “osteoarthritic pain” means joint pain in an osteoarthritic joint.

The phrase “rheumatoid arthritic pain” means joint pain in a rheumatoid arthritic joint.

The phrase “inflammatory pain” means pain due to edema or swelling of any inflamed tissue, including inflammatory joint pain. Inflammatory joint pain includes rheumatoid arthritic pain.

The phrase “mechanical pain” means a pain related to an injury or damage to a structure of a body, including osteoarthritic pain, surgical pain, burn pain, bone cancer pain, and the like.

The phrase “acute pain” means any pain, including, but not limited to, joint pain, osteoarthritic pain, rheumatoid arthritic pain, inflammatory pain, pain from a burn, pain from a cut, surgical pain, pain from fibromyalgia, bone cancer pain, menstrual pain, back pain, headache, static allodynia, and dynamic allodynia, that lasts from 1 minute to 91 days, 1 minute to 31 days, 1 minute to 7 days, 1 minute to 5 days, 1 minute to 3 days, 1 minute to 2 days, 1 hour to 91 days, 1 hour to 31 days, 1 hour to 7 days, 1 hour to 5 days, 1 hour to 3 days, 1 hour to 2 days, 1 hour to 24 hours, 1 hour to 12 hours, or 1 hour to 6 hours, per occurrence if left untreated. Acute pain includes, but is not limited to, joint pain, osteoarthritic pain, rheumatoid arthritic pain, inflammatory pain, pain from a burn, pain from a cut, surgical pain, pain from fibromyalgia, bone cancer pain, menstrual pain, back pain, headache, static allodynia, dynamic allodynia, acute joint pain, acute osteoarthritic pain, acute rheumatoid arthritic pain, acute inflammatory pain, acute headache, acute menstrual pain, acute back pain, and acute pain from fibromyalgia. Acute pain may be selected from acute joint pain, acute osteoarthritic pain, acute rheumatoid arthritic pain, acute inflammatory pain, acute headache, acute menstrual pain, and acute back pain. Acute pain may be selected from acute joint pain, acute osteoarthritic pain, acute rheumatoid arthritic pain, and acute inflammatory pain. Acute pain may be selected from acute joint pain, acute osteoarthritic pain, and acute rheumatoid arthritic pain. Acute pain may be selected from acute joint pain and acute osteoarthritic pain.

It should be appreciated that alleviating acute pain means having an appreciable pain alleviating effect within 91, 31, 7, 5, 3, or 2 days, or 24, 12, 6, 3, 2, 1, 0.5, 0.25, 0.20. 0.17, or 0.10 hours after administering the first dose of an active compound or combination comprising the substituted dialkyl ether, substituted aryl-alkyl ether, substituted dialkyl thioether, substituted dialkyl ketone, or substituted-alkyl compound and another active ingredient.

The phrase “chronic pain” means any pain, including, but not limited to, joint pain, osteoarthritic pain, rheumatoid arthritic pain, inflammatory pain, pain from a burn, pain from a cut, surgical pain, pain from fibromyalgia, bone cancer pain, menstrual pain, back pain, headache, static allodynia, dynamic allodynia, chronic joint pain, chronic osteoarthritic pain, chronic rheumatoid arthritic pain, chronic inflammatory pain, chronic headache, chronic back pain, and chronic pain from fibromyalgia that lasts longer than 91 days, 6 months, 1 year, 5 years, or 10 years per occurrence if left untreated. Chronic pain may be selected from chronic joint pain, chronic osteoarthritic pain, chronic rheumatoid arthritic pain, chronic inflammatory pain, chronic headache, chronic back pain, and chronic pain from fibromyalgia. Chronic pain may be selected from chronic joint pain, chronic osteoarthritic pain, chronic rheumatoid arthritic pain, chronic inflammatory pain, chronic headache, and chronic back pain. Chronic pain may be selected from chronic joint pain, chronic osteoarthritic pain, chronic rheumatoid arthritic pain, and chronic inflammatory pain. Chronic pain may be selected from chronic joint pain, chronic osteoarthritic pain, and chronic rheumatoid arthritic pain. Chronic pain may be selected from chronic joint pain and chronic osteoarthritic pain.

It should be appreciated that alleviating chronic pain means having an appreciable pain alleviating effect within 91, 60, 31, 28, 21, 14, 7, 3, or 2 days or 24, 12, 6, 3, 2, 1, 0.5, 0.25, 0.20. 0.17, or 0.10 hours after administering the first dose of an active compound or other active ingredient.

The phrase “wherein the pain is mediated by IL-6, IL-6 sR, or IL-6 receptor” refers to pain in a patient that is alleviated by administering to the patient an inhibitor of the cytokine interleukin-6 (“IL-6”), its cell-bound receptor interleukin-6 receptor (“IL-6 receptor”), or interleukin-6 soluble receptor (“IL-6sR”), which is an unbound cleavage fragment of IL-6 receptor that can bind to IL-6. An inhibitor of pain mediated by IL-6, IL-6sR, or IL-6 receptor may also be identified by practicing the method of Biological Method 5 below. An IL-6, IL-6sR, or IL-6 receptor inhibitor includes inhibitors of IL-6, IL-6sR, or IL-6 expression or biological activity and promoters of IL-6, IL-6sR, or IL-6 clearance, respectively.

It should be appreciated that pain substantially (>10%) resulting from a deficit of oxygen in an organ (e.g., brain, heart, or liver) is not embraced by any pain disclosed herein.

The phrases “therapeutically effective amount” and “effective amount” are synonymous and mean an amount of a compound or a combination as described above sufficient to alleviate, eliminate, inhibit or prevent the onset, or inhibit the progress, prevent further progress, or reverse progression, in part or in whole, of any one or more pathological hallmarks or symptoms of the disease or disorder that is appreciated or suspected or expected in the particular patient being treated.

It should be appreciated that a therapeutically effective or effective amount means an amount sufficient to have a desired effect in a patient to whom that amount has been administered. For illustrative example, where cartilage damage is being inhibited, a therapeutically effective amount includes a cartilage damage inhibiting effective amount. Where osteoarthritis is being treated, a therapeutically effective amount includes an osteoarthritis treating effective amount. Where pain is being alleviated, a therapeutically effective amount includes a pain alleviating effective amount. Where osteoarthritic or rheumatoid arthritic pain is being alleviated, a therapeutically effective amount includes an osteoarthritic or rheumatoid arthritic pain alleviating effective amount, respectively.

It should be appreciated that endothelin-1 is a member of a family of 21-residue peptides. The endothelin peptides are produced by a vast array of cell types in response to diverse stimuli, and are mediated through specific ET_{A} and ET_{B} receptors. Receptors of the ET_{A} type exhibit higher affinity for endothelin-1 than endothelin-3, and can be selectively blocked by antagonists known in the art.

An active compound having an anti-inflammatory, an analgesic, anti-arthritic, or a cartilage damage inhibiting effect, or any combination of these effects, may be readily identified by one of ordinary skill in the pharmaceutical or medical arts by assaying the substituted dialkyl ether, substituted aryl-alkyl ether, substituted dialkyl thioether, substituted dialkyl ketone, or substituted-alkyl compound in any number of well known assays for measuring determining the substituted dialkyl ether, substituted aryl-alkyl ether, substituted dialkyl thioether, substituted dialkyl ketone, or substituted-alkyl compound's effects on cartilage damage, arthritis, inflammation, or pain. These assays include in vitro assays that utilize cartilage samples and in vivo assays in whole animals that measure cartilage degradation, inhibition of inflammation, or pain alleviation.

For example with regard to assaying cartilage damage in vitro, an amount of an active compound or control vehicle may be administered with a cartilage damaging agent to cartilage, and the cartilage damage inhibiting effects in both tests studied by gross examination or histopathologic examination of the cartilage, or by measurement of biological markers of cartilage damage such as, for example, proteoglycan content or hydroxyproline content. Further, in vivo assays to assay cartilage damage may be performed as follows: an amount of an active compound or control vehicle may be administered with a cartilage damaging agent to an animal, and the effects of the substituted dialkyl ether, substituted aryl-alkyl ether, substituted dialkyl thioether, substituted dialkyl ketone, or substituted-alkyl compound being assayed on cartilage in the animal may be evaluated by gross examination or histopathologic examination of the cartilage, by observation of the effects in an acute model on functional limitations of the affected joint that result from cartilage damage, or by measurement of biological markers of cartilage damage such as, for example, proteoglycan content or hydroxyproline content.

Several methods of identifying an active compound with cartilage damage inhibiting properties are described below. The amount to be administered in an assay is dependent upon the particular assay employed, but in any event is not higher than the well known maximum amount of a compound that the particular assay can effectively accommodate.

Similarly, substituted dialkyl ether, substituted aryl-alkyl ether, substituted dialkyl thioether, substituted dialkyl ketone, or substituted-alkyl compounds having pain-alleviating properties may be identified using any one of a number of in vivo animal models of pain. A number of in vivo animal models of joint pain are known in the art, and a model of endothelin-1 mediated pain is described by Piovezan, Anna P., et al., British Journal of Pharmacology, 2000;129:961-968, which is incorporated herein by reference.

Still similarly, substituted dialkyl ether, substituted aryl-alkyl ether, substituted dialkyl thioether, substituted dialkyl ketone, or substituted-alkyl compounds having anti-inflammatory properties may be identified using any one of a number of in vivo animal models of inflammation. For example, for an example of inflammation models, see U.S. Pat. No. 6,329,429, which is incorporated herein by reference.

Still similarly, substituted dialkyl ether, substituted aryl-alkyl ether, substituted dialkyl thioether, substituted dialkyl ketone, or substituted-alkyl compounds having anti-arthritic properties may be identified using any one of a number of in vivo animal models of arthritis. For example, for an example of arthritis models, see also U.S. Pat. No. 6,329,429.

In a clinical setting, a physician may assess a patients need for, or response to, treatment of osteoarthritis, rheumatoid arthritis, impaired joint function, pain, including osteoarthritic pain, rheumatoid arthritic pain, acute pain, joint pain, chronic pain, inflammatory pain, pain mediated by IL-6, IL-6sR, or IL-6 receptor, or mechanical pain by administering a standard assessment questionnaire such as WOMAC or the Patient Global Impression of Change (“PGIC”).

Further with respect to the assessment of a patients need for, or response to, treatment of the aforementioned pain states and bone cancer pain, pain mediated by endothelin-1, static allodynia, and dynamic allodynia, the physician may apply a pain assessment scale such as the Visual Analog Scale (“VAS”), wherein a patient is asked to indicate a point on a 100 millimeter line, having a left anchor of no pain and a right anchor of worst possible pain, corresponding to their degree of pain or the Likert score, wherein a patient is asked to categorize their pain on a numerical scale of from 0 (no pain) to 10 (worst possible pain).

Compounds useful in an invention method, composition, or combination may be formulated alone or with one or more other therapeutic agents which are to form the intended combination, including wherein said different drugs have varying half-lives, by creating controlled-release forms of said drugs with different release times which achieves relatively uniform dosing; or, in the case of non-human patients, a medicated feed dosage form in which said drugs used in the combination are present together in admixture in the feed composition. There is further provided herein a method of co-administration in which the combination of drugs is achieved by the simultaneous, or non-simultaneous, sequential or concurrent, administration of said drugs to be given in combination; including co-administration by means of different dosage forms and routes of administration; or the use of combinations in accordance with different but regular and continuous dosing schedules whereby desired plasma levels of said drugs involved are maintained in the patient being treated, which may be achieved even though the individual drugs making up said combination are not being administered to said patient simultaneously.

In determining what constitutes a therapeutically effective amount of an active compound, or a pharmaceutically acceptable salt thereof, or a combination of the same with a selective COX-2 inhibitor, for alleviating pain, preventing or treating osteoarthritis, preventing or treating rheumatoid arthritis, improving joint function, or preventing or inhibiting cartilage damage according to the invention method, a number of factors will generally be considered by the medical practitioner or veterinarian in view of the experience of the medical practitioner or veterinarian, published clinical studies, the subject's (ie, mammal's) age, sex, weight and general condition, as well as the type and extent of the disease, disorder or condition being treated, and the use of other medications, if any, by the subject. Such amounts will generally be from about 0.1 mg/kg to about 300 mg/kg of subject body weight. Typical doses will be from about 10 to about 5000 mg/day for an adult subject of normal weight. In a clinical setting, regulatory agencies such as, for example, the FDA in the United States may require a particular therapeutically effective amount.

As such, the administered dose may fall within the ranges or amounts recited above, or may vary outside, ie, either below or above, those ranges depending upon the requirements of the individual subject, the severity of the condition being treated, and the particular therapeutic formulation being employed. Determination of a proper dose for a particular situation is within the skill of the medical or veterinary arts. Generally, treatment may be initiated using smaller dosages of an active compound useful in the invention method, or a pharmaceutically acceptable salt thereof, or a combination of the same with another therapeutic agent, that are less than optimum for a particular subject. Thereafter, the dosage can be increased by small increments until the optimum effect under the circumstance is reached. For convenience, the total daily dosage may be divided and administered in portions during the day, if desired.

The invention method may be conducted by administering an active compound useful in the invention method, or a pharmaceutically acceptable salt thereof, or a combination of the same with another therapeutic agent, either alone or formulated in a composition suitable for pharmaceutical administration. Pharmaceutical compositions, described here briefly and more fully below, of an active compound useful in the invention method, or a pharmaceutically acceptable salt thereof, are produced by formulating the active compound in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses.

Some examples of suitable pharmaceutical carriers, including pharmaceutical diluents, are gelatin capsules; sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The compositions to be employed in the invention can also contain other components such as coloring agents, flavoring agents, and/or preservatives. These materials, if present, are usually used in relatively small amounts. The compositions can, if desired, also contain other therapeutic agents commonly employed to treat osteoarthritis. Further, the compositions can, if desired, also contain other therapeutic agents commonly employed to treat secondary symptoms such as, for example, inflammation or pain that may or may not accompany cartilage damage. For example, the compositions may contain aspirin, naproxen, or similar anti-inflammatory analgesic agents.

The percentage of the active ingredients in the foregoing compositions can be varied within wide limits, but for practical purposes may be present in a concentration of at least 10% in a solid composition and at least 2% in a primary liquid composition, both up to about 95%.

Typical routes of administration of an active compound useful in the invention method, or a pharmaceutically acceptable salt thereof, or a combination of the same with another therapeutic agent, are oral or parenteral. For example, a useful intravenous dose is between 5 and 50 mg, and a useful oral dosage is between 20 and 800 mg. The dosage is within the dosing range used in treatment of diseases resulting in cartilage damage, loss of joint function, or pain such as rheumatoid arthritis and osteoarthritis, or as would be determined by the physician according to the needs of the patient as described above.

The substituted dialkyl ether, substituted aryl-alkyl ether, substituted dialkyl thioether, substituted dialkyl ketone, or substituted-alkyl compound useful in the invention method, or a pharmaceutically acceptable salt thereof, or a combination of the same with another therapeutic agent as described above, may be administered in any form, including unit dosage form. A unit dosage form of the an active compound useful in the invention method, or a pharmaceutically acceptable salt thereof, to be used in this invention may also comprise other compounds useful in the therapy of diseases resulting in cartilage damage or loss of joint function.

The advantages of the instant invention include the relatively nontoxic nature of the substituted dialkyl ether, substituted aryl-alkyl ether, substituted dialkyl thioether, substituted dialkyl ketone, or substituted-alkyl compounds useful in the invention method, their ease of preparation, the fact that the substituted dialkyl ether, substituted aryl-alkyl ether, substituted dialkyl thioether, substituted dialkyl ketone, or substituted-alkyl compounds are well-tolerated, and the ease of IV and oral administration of the drugs.

Another important advantage is that the substituted dialkyl ether, substituted aryl-alkyl ether, substituted dialkyl thioether, substituted dialkyl ketone, or substituted-alkyl compounds useful in the invention method represent a new mechanistic treatment of arthritic pain and cartilage damage. This will be important to patients given the efficacy and/or safety limitations of current pharmacological treatments for OA, OA pain, RA pain, cartilage damage, and the like as described above. For example, it was found that 6-(5-carboxy-5-methyl-hexyloxy)-2,2-dimethyl-hexanoic acid, calcium salt does not inhibit prostaglandin-F2a (“PGF2a”) expression from calcium ionophore-stimulated rat basophilic leukemia cells or leukotriene B4 (“LTB4”) expression from calcium ionophore-stimulated rat basophilic leukemia cells, and thus will not exhibit the gastrointestinal liabilities (e.g., gastric ulceration and bleeding, dyspepsia, etc.) of cyclooxygenase inhibitors such as naproxen or indomethacin or 5-lipoxygenase inhibitors.

Another important advantage is that the substituted dialkyl ether, substituted aryl-alkyl ether, substituted dialkyl thioether, substituted dialkyl ketone, or substituted-alkyl compounds useful in the invention method do not elevate blood pressure, which has been observed in some patients for the arthritic pain alleviating agent VIOXX®. For example, it was found that 6-(5-carboxy-5-methyl-hexyloxy)-2,2-dimethyl-hexanoic acid, calcium salt has no effect or perhaps even slightly (1-5 mmHg) decreases blood pressure in normal healthy humans.

Another important advantage is that the substituted dialkyl ether, substituted aryl-alkyl ether, substituted dialkyl thioether, substituted dialkyl ketone, or substituted-alkyl compounds useful in the invention method provide disease modifying activity for osteoarthritis and other diseases and disorders having cartilage damage as a component of their pathology. There is currently no recognized drug on the market that is approved for this treatment effect. Even further, this cartilage damage inhibiting effect and the OA and RA symptom (pain) treating effect are now found in one pharmacological agent, and the instant substituted dialkyl ether, substituted aryl-alkyl ether, substituted dialkyl thioether, substituted dialkyl ketone, or substituted-alkyl compound may be administered once per day. If patients can take one drug instead of two and only one pill per day instead of two or more, patient compliance, and thus patient benefit, goes up.

Further, the instant invention may, if desired, allow the amount of an anti-inflammatory agent and/or pain relieving agent currently used in the treatment of patients suffering from cartilage damage and inflammation and/or pain to be reduced or even eliminated. It is known that anti-inflammatory and analgesic agents may produce undesirable side effects such as gastro-intestinal bleeding and ulceration. These side effects may be avoided, reduced or eliminated by using the instant invention to inhibit cartilage damage.

Intermediates for the synthesis of an active compound useful in the invention method, or a pharmaceutically acceptable salt thereof, useful in the invention method, and pharmaceutically acceptable salts thereof, may be prepared by one of ordinary skill in the art of organic chemistry by adapting various synthetic procedures that are well-known in the art of organic chemistry. These synthetic procedures may be found in the literature in, for example, Reagents for Organic Synthesis, by Fieser and Fieser, John Wiley & Sons, Inc, New York, 2000; Comprehensive Organic Transformations, by Richard C. Larock, VCH Publishers, Inc, New York, 1989; the series Compendium of Organic Synthetic Methods,1989, by Wiley-Interscience; the text Advanced Organic Chemistry, 4^{th} edition, by Jerry March, Wiley-Interscience, New York, 1992; or the Handbook of Heterocyclic Chemistry by Alan R. Katritzky, Pergamon Press Ltd, London, 1985, to name a few. Alternatively, a skilled artisan may find methods useful for preparing the intermediates in the chemical literature by searching widely available databases such as, for example, those available from the Chemical Abstracts Service, Columbus, Ohio, or MDL Information Systems GmbH (formerly Beilstein Information Systems GmbH), Frankfurt, Germany.

Preparations of the compounds useful in an invention method, composition, or combination may use starting materials, reagents, solvents, and catalysts that may be purchased from commercial sources or they may be readily prepared by adapting procedures in the references or resources cited above. Commercial sources of starting materials, reagents, solvents, and catalysts useful in preparing substituted dialkyl ether, substituted aryl-alkyl ether, substituted dialkyl thioether, substituted dialkyl ketone, or substituted-alkyl compounds include, for example, The Aldrich Chemical Company, and other subsidiaries of Sigma-Aldrich Corporation, St. Louis, Mo., BACHEM, BACHEM A.G., Switzerland, or Lancaster Synthesis Ltd, United Kingdom.

Syntheses of some compounds useful in an invention method, composition, or combination may utilize starting materials, intermediates, or reaction products that contain a reactive functional group. During chemical reactions, a reactive functional group may be protected using protecting groups that render the reactive group substantially inert to the reaction conditions employed. A protecting group is introduced onto a starting material prior to carrying out the reaction step for which a protecting group is needed. Once the protecting group is no longer needed, the protecting group can be removed. It is well within the ordinary skill in the art to introduce protecting groups during a synthesis of an active compound, or a pharmaceutically acceptable salt thereof, and then later remove them. Procedures for introducing and removing protecting groups are known and referenced such as, for example, in Protective Groups in Organic Synthesis, 2^{nd} ed., Greene T. W. and Wuts P. G., John Wiley & Sons, New York: N.Y., 1991, which is hereby incorporated by reference. Thus, for example, protecting groups such as the following may be utilized to protect amino, hydroxyl, and other groups: carboxylic acyl groups such as, for example, formyl, acetyl, and trifluoroacetyl; alkoxycarbonyl groups such as, for example, ethoxycarbonyl, tert-butoxycarbonyl (BOC), β,β,β-trichloroethoxycarbonyl (TCEC), and β-iodoethoxycarbonyl; aralkyloxycarbonyl groups such as, for example, benzyloxycarbonyl (CBZ), para-methoxybenzyloxycarbonyl, and 9-fluorenylmethyloxycarbonyl (FMOC); trialkylsilyl groups such as, for example, trimethylsilyl (TMS) and tert-butyldimethylsilyl (TBDMS); and other groups such as, for example, triphenylmethyl (trityl), tetrahydropyranyl, vinyloxycarbonyl, ortho-nitrophenylsulfenyl, diphenylphosphinyl, para-toluenesulfonyl (Ts), mesyl, trifluoromethanesulfonyl, and benzyl. Examples of procedures for removal of protecting groups include hydrogenolysis of CBZ groups using, for example, hydrogen gas at 50 psi in the presence of a hydrogenation catalyst such as 10% palladium on carbon, acidolysis of BOC groups using, for example, hydrogen chloride in dichloromethane, trifluoroacetic acid (TFA) in dichloromethane, and the like, reaction of silyl groups with fluoride ions, and reductive cleavage of TCEC groups with zinc metal.

Preparations of an active compound useful in the invention method, or a pharmaceutically acceptable salt thereof, are incorporated by reference to the patents and patent application publications incorporated by reference above.

It should be appreciated that in the below studies, a dose in mg/kg means the weight in milligrams of test compound per weight of test animal body weight in kilograms.

It should be appreciated that in Biological Methods 1, 2, 4, and 5 below, hind paw weight bearing differentials between the paw of the limb having the control joint (in animals administered test compound vehicle only) and the paw of the limb having the contralateral test joint (in control or inducement animals administered test compound vehicle only and in treatment animals administered test compound dissolved in test compound vehicle) were determined using an incapacitance tester, model 2KG (Linton Instrumentation, Norfolk, United Kingdom). The incapacitance tester has a chamber on top with an outwardly sloping front wall that supports a rat's front limbs, and two weight sensing pads, one for each hind paw, that facilitates this determination. The hind paw weight bearing differentials were expressed in grams and calculated as follows: (the average weight in grams placed on the control limb minus the average weight in grams placed on the contralateral test limb for each of the inducement or treatment animals) minus (the average weight in grams placed on the control limb minus the average weight in grams placed on the contralateral test limb for each of the control animals). All results were statistically analyzed by comparing the average results for an induction or treatment group with the average result for its control group at the same time point using analysis of covariance (“ANCOVA”) followed by the Hoehberg's procedure and those with statistical significance had ρ<0.05 unless otherwise noted.

It should be appreciated that it is not necessary to use ANCOVA followed by Hochberg's procedure to statistically analyze data in a method of this invention. Alternative statistical analyses are known that may be used such as ANCOVA without Hochberg's procedure, analysis of variance (“ANOVA”) with Hochberg's procedure, ANOVA without Hochberg's procedure, t-test with Hochberg's procedure, and t-test without Hochberg's procedure.

It should also be appreciated that the 1L-6 and IL-6sR used as described in all of the below examples was the commercially available recombinant human IL-6 and human IL-6sR purchased from R&D Systems, Minneapolis, Minn.

It should also be appreciated that prior to injection of MIA in Biological Methods 1, 2, and 4 or IL-6 plus IL-6sR in Biological Method 5, rats were anesthetized with 5% volume/volume (“v/v”) isoflurane gas until knocked down and maintained with 2% v/v isoflurane. Rats became fully conscious about 5 minutes after isoflurane administration was discontinued.

It should also be appreciated that in Biological Method 5, the cytokine vehicle comprised 0.5% HPMC plus 0.2% Tween 80 in water (“HPMC/Tween 80”).

It will be shown below in Biological Method 5 that administration of 6-(5-carboxy-5-methyl-hexyloxy)-2,2-dimethyl-hexanoic acid, calcium salt will inhibit (IL-6 plus IL-6sR)-induced pain.

The newly discovered ability of a substituted dialkyl ether, substituted aryl-alkyl ether, substituted dialkyl thioether, substituted dialkyl ketone, or substituted-alkyl compound, or a pharmaceutically acceptable salt thereof, to inhibit cartilage damage, alleviate pain, and treat osteoarthritis has been established in animal models as described below.

### Biological Method 1

Monosodium Iodoacetate-Induced Osteoarthritis in Rat Model of Cartilage Damage (“MIA Rat”):

One end result of the induction of osteoarthritis in this model, as determined by histologic analysis, is the development of an osteoarthritic condition within the affected joint, as characterized by the loss of Toluidine blue staining and formation of osteophytes. Associated with the histologic changes is a concentration-dependent degradation of joint cartilage, as evidenced by affects on hind-paw weight distribution of the limb having the affected joint, the presence of increased amounts of proteoglycan or hydroxyproline in the joint upon biochemical analysis, or histopathological analysis of the osteoarthritic lesions. The hind-paw weight distribution effects reported below, or the effects that would be expected to be observed, for the substituted dialkyl ether, substituted aryl-alkyl ether, substituted dialkyl thioether, substituted dialkyl ketone, or substituted-alkyl compounds useful in the invention method, or a pharmaceutically acceptable salt thereof, result from, or would result from, the ability of substituted dialkyl ether, substituted aryl-alkyl ether, substituted dialkyl thioether, substituted dialkyl ketone, or substituted-alkyl compounds useful in the invention method, or a pharmaceutically acceptable salt thereof, to directly inhibit damage to articular cartilage.

Generally, In the MIA Rat model on Day 0, the hind-paw weight differential between the right arthritic joint and the left healthy joint of male Wistar rats (150 g) are determined with an incapacitance tester, model 2KG (Linton Instrumentation, Norfolk, United Kingdom). The incapacitance tester has a chamber on top with an outwardly sloping front wall that supports a rat's front limbs, and two weight sensing pads, one for each hind paw, that facilitates this determination. Then the rats are anesthetized with isofluorine, and the right, hind leg knee joint is injected with 1.0 mg of mono-iodoacetate (“MIA”) through the infrapatellar ligament. Injection of MIA into the joint results in the inhibition of glycolysis and eventual death of surrounding chondrocytes. The rats are further administered either an active compound useful in the invention method, or a pharmaceutically acceptable salt thereof, or vehicle (in the instant case, water) daily for 14 days or 28 days. The substituted dialkyl ether, substituted aryl-alkyl ether, substituted dialkyl thioether, substituted dialkyl ketone, or substituted-alkyl compound useful in the invention method, or a pharmaceutically acceptable salt thereof, is typically administered at a dose of 30 mg of substituted dialkyl ether, substituted aryl-alkyl ether, substituted dialkyl thioether, substituted dialkyl ketone, or substituted-alkyl compound useful in the invention method, or a pharmaceutically acceptable salt thereof, per kilogram of rat per day (30 mg/kg/day), but may be administered at other doses such as, for example, 10 mg/kg/day, 60 mg/kg/day, 90-mg/kg/day, or 100 mg/kg/day according to the requirements of the compound being studied. It is well within the level of ordinary skill in the pharmaceutical arts to determine a proper dosage of an active compound useful in the invention method, or a pharmaceutically acceptable salt thereof, in this model. Administration of the substituted dialkyl ether, substituted aryl-alkyl ether, substituted dialkyl thioether, substituted dialkyl ketone, or substituted-alkyl compound useful in the invention method, or a pharmaceutically acceptable salt thereof, in this model is optionally by oral administration or intravenous administration via an osmotic pump. After 7 and 14 days for a two week study, or 7, 14, and 28 days for a four week study, the hind-paw weight distribution is again determined. Typically, the animals administered vehicle alone place greater weight on their unaffected left hind paw than on their right hind paw, while animals administered an active compound useful in the invention method, or a pharmaceutically acceptable salt thereof, show a more normal (i.e., more like a healthy animal) weight distribution between their hind paws. This change in weight distribution is proportional to the degree of joint cartilage damage. Percent inhibition of a change in hind paw joint function is calculated as the percent change in hind-paw weight distribution for treated animals versus control animals. For example, for a two week study,

Percent inhibition of a change in hind paw weight distribution

$$\begin{matrix}Percent inhibition of a change in \\ hind paw weight distribution\end{matrix} = \{ 1 - [ \frac{( Δ {W}_{G} )}{( Δ {W}_{C} )} ] \} \times 100$$

wherein:

ΔW_{C} is the hind-paw weight differential between the healthy left limb and the arthritic limb of the control animal administered vehicle alone, as measured on Day 14; and

ΔW_{G} is the hind-paw weight differential between the healthy left limb and the arthritic limb of the animal administered an active compound useful in the invention method, or a pharmaceutically acceptable salt thereof, as measured on Day 14.

In order to measure biochemical or histopathological end points in the MIA Rat model, some of the animals in the above study may be sacrificed, and the amounts of free proteoglycan in both the osteoarthritic right knee joint and the contralateral left knee joint may be determined by biochemical analysis. The amount of free proteoglycan in the contralateral left knee joint provides a baseline value for the amount of free proteoglycan in a healthy joint. The amount of proteoglycan in the osteoarthritic right knee joint in animals administered an active compound useful in the invention method, and the amount of proteoglycan in the osteoarthritic right knee joint in animals administered vehicle alone, are independently compared to the amount of proteoglycan in the contralateral left knee joint. The amounts of proteoglycan lost in the osteoarthritic right knee joints are expressed as percent loss of proteoglycan compared to the contralateral left knee joint control. The percent inhibition of proteoglycan loss, may be calculated as {1−[(proteoglycan loss from joint (%) with vehicle)−(proteoglycan loss from joint with substituted dialkyl ether, substituted aryl-alkyl ether, substituted dialkyl thioether, substituted dialkyl ketone, or substituted-alkyl compound useful in the invention method)]÷(proteoglycan loss from joint (%) with vehicle)}×100.

The MIA Rat data that are expected from the analysis of proteoglycan loss would establish that substituted dialkyl ether, substituted aryl-alkyl ether, substituted dialkyl thioether, substituted dialkyl ketone, or substituted-alkyl compounds useful in the invention method, including a pharmaceutically acceptable salt thereof, including a compound named 6-(5-carboxy-5-methyl-hexyloxy)-2,2-dimethyl-hexanoic acid, calcium salt, are effective for inhibiting cartilage damage, improving joint function, and treating osteoarthritis in mammalian patients, including human. The compound named 6-(5-carboxy-5-methyl-hexyloxy)-2,2-dimethyl-hexanoic acid, calcium salt was tested in MIA, and the results are described below in Biological Method 2.

### Biological Method 2

6-(5-Carboxy-5-methyl-hexyloxy)-2,2-dimethyl-hexanoic Acid, Calcium Salt in MIA:

In a particular experiment, monosodium iodoacetate (“MIA”) (1 mg/joint) was injected through the infrapatellar ligament of the right knee of anesthetized male, Wistar rats. The contralateral control knee was injected with 50 μL of physiologic saline. The change in hind paw weight distribution, as determined by use of an incapacitance tester, between the right (arthritic) and left (contralateral control) knees was utilized as an index of functional limitation in the arthritic knee. Limitations in joint function were determined on Days 7, 14, and 28 following induction of arthritis. Following sacrifice, erosion severity was determined on the tibial plateaus from the arthritic joint. Histological analysis was also conducted on these samples. The basis of the invention is derived from the ability of 6-(5-carboxy-5-methyl-hexyloxy)-2,2-dimethyl-hexanoic acid, calcium salt, dosed orally two times per day (i.e., PO; BID), to significantly decrease cartilage erosion severity at 30-mg/kg and 10-mg/kg doses and by its ability to decrease joint function limitations as defined by a reduction in differential hind-limb weight bearing.

For oral administration, 6-(5-carboxy-5-methyl-hexyloxy)-2,2-dimethyl-hexanoic acid, calcium salt, was dissolved in double distilled water (all calculations are based on the percent parent of the drug). Dose-response studies at doses of 3-, 10-, and 30-mg/kg [peroral (“PO”); twice a day (“BID”)] demonstrated that 6-(5-carboxy-5-methyl-hexyloxy)-2,2-dimethyl-hexanoic acid, calcium salt, at 4 weeks post-MIA, significantly decreased the degree of structural damage to the cartilage at the 30 mg/kg dose and significantly decreased joint pain.

The results of these studies with oral dosing are shown below in Table 1 in the columns labelled “IJFL (%+/−SEM)”, wherein LTFL means Inhibition of Joint Function Limitation and SEM means standard error of the mean; “SDCES”, wherein SDCES means Significant Decrease In Cartilage Erosion Severity, and “Decreased Erosion Size”, wherein Decreased Erosion Size means whether or not there was a statistically significant decrease in the size of the area of joint erosion.

TABLE 1
Four week study with oral administration of CI-1027,
two times per day per dose:

| Dose | IJFL | | Decreased |
|---|---|---|---|
| (mg/kg) | (% +/− SEM) | SDCES^{b} | Erosion Size |
| 30 | 63 +/− 8^{a} | Yes^{d} | No |
| 10 | 36 +/− 9 | Yes^{e} | Yes^{c} |
| 3 | 3 +/− 156 | Yes^{f} | No |
| 30 | 60 +/− 8^{a} | No^{g} | No |

^{a}p < 0.05 versus vehicle (One-way Analysis of Variance (“One Way ANOVA”; Dunnett's Multiple Comparison Procedure);
^{b}p < 0.05 versus vehicle (Ridit Analysis);
^{c}p < 0.05 versus vehicle (One Way ANOVA);
^{d}actual p = 0.027;
^{e}actual p = 0.001;
^{f}actual p = 0.043.
^{g}actual p = 0.100

The ridit analysis was used to determine differences in overall erosion severity. This parameter takes into account both the erosion grade (0=no erosion, I=erosion extending into the superficial or middle layers, or II=deep layer erosion), and area (small, medium and large, quantified by dividing the area of the largest erosion in each score into thirds) simultaneously. The analysis recognizes that each unit of severity is different, but does not assume a mathematical relationship between units.

The MIA Rat data reported above in Table 1 establish that substituted dialkyl ether, substituted aryl-alkyl ether, substituted dialkyl thioether, substituted dialkyl ketone, or substituted-alkyl compounds useful in the invention method, or a pharmaceutically acceptable salt thereof, including a compound named 6-(5-carboxy-5-methyl-hexyloxy)-2,2-dimethyl-hexanoic acid, calcium salt, are effective at preventing or inhibiting cartilage damage, improving joint function, alleviating joint pain, and preventing or treating osteoarthritis.

The compound 6-(5-carboxy-5-methyl-hexyloxy)-2,2-dimethyl-hexanoic acid, calcium salt may also be administered subcutaneously via osmotic pumps. Dosing may be carried out at, for example, 100-mg/kg/day, 90-mg/kg/day, 30-mg/kg/day, and 10-mg/kg/day dosing.

### Biological Method 3

Induction of Experimental Osteoarthritis in Rabbit (“EOA in Rabbit”):

Normal rabbits are anaesthetized and anteromedial incisions of the right knees performed. The anterior cruciate ligaments are visualized and sectioned. The wounds are closed and the animals are housed in individual cages, exercised, and fed ad libitum. Rabbits are given either vehicle (water) or 6-(5-carboxy-5-methyl-hexyloxy)-2,2-dimethyl-hexanoic acid, calcium salt, or a pharmaceutically acceptable salt thereof, (10 rabbits per group). Each group is dosed three times per day with the 6-(5-carboxy-5-methyl-hexyloxy)-2,2-dimethyl-hexanoic acid, calcium salt, or a pharmaceutically acceptable salt thereof, group receiving 30-mg/kg/dose or 10-mg/kg/dose. The rabbits are euthanized 8 weeks after surgery and the proximal end of the tibia and the distal end of the femur are removed from each animal.

Macroscopic Grading

The cartilage changes on the femoral condyles and tibial plateaus are graded separately under a dissecting microscope (Stereozoom, Bausch & Lomb, Rochester, N.Y.). The depth of erosion is graded on a scale of 0 to 4 as follows: grade 0=normal surface; Grade 1=minimal fibrillation or a slight yellowish discoloration of the surface; Grade 2=erosion extending into superficial or middle layers only; Grade 3=erosion extending into deep layers; Grade 4=erosion extending to subchondral bone. The surface area changes are measured and expressed in mm². Representative specimens may also be used for histologic grading (see below).

Histologic Grading

Histologic evaluation is performed on sagittal sections of cartilage from the lesional areas of the femoral condyle and tibial plateau. Serial sections (5 um) are prepared and stained with safranin-O. The severity of OA lesions is graded on a scale of 0-14 by two independent observers using the histologic-histochemical scale of Mankin et al. This scale evaluates the severity of OA lesions based on the loss of safranin-O staining (scale 0-4), cellular changes (scale 0-3), invasion of tidemark by blood vessels (scale 0-1) and structural changes (scale 0-6). On this latter scale, 0 indicates normal cartilage structure and 6 indicates erosion of the cartilage down to the subchondral bone. The scoring system is based on the most severe histologic changes in the multiple sections.

Representative specimens of synovial membrane from the medial and lateral knee compartments are dissected from underlying tissues. The specimens are fixed, embedded, and sectioned (5 um) as above, and stained with hematoxylin-eosin. For each compartment, two synovial membrane specimens are examined for scoring purposes and the highest score from each compartment is retained. The average score is calculated and considered as a unit for the whole knee. The severity of synovitis is graded on a scale of 0 to 10 by two independent observers, adding the scores of 3 histologic criteria: synovial lining cell hyperplasia (scale 0-2); villous hyperplasia (scale 0-3); and degree of cellular infiltration by mononuclear and polymorphonuclear cells (scale 0-5): 0 indicates normal structure.

Statistical Analysis

Mean values and SEM are calculated and statistical analysis is done using the Mann-Whitney U-test.

If these EOA studies were performed with a substituted dialkyl ether, substituted aryl-alkyl ether, substituted dialkyl thioether, substituted dialkyl ketone, or substituted-alkyl compound, the results would show that the active compounds, or a pharmaceutically acceptable salt thereof, including a compound named 6-(5-carboxy-5-methyl-hexyloxy)-2,2-dimethyl-hexanoic acid, calcium salt, reduce the size of the lesion on the tibial plateaus, and perhaps the damage in the tibia or on the femoral condyles. In conclusion, the EOA results would show that substituted dialkyl ether, substituted aryl-alkyl ether, substituted dialkyl thioether, substituted dialkyl ketone, or substituted-alkyl compounds useful in the invention method, or a pharmaceutically acceptable salt thereof, including a compound named 6-(5-carboxy-5-methyl-hexyloxy)-2,2-dimethyl-hexanoic acid, calcium salt, have significant inhibition effects on the damage to cartilage.

### Biological Method 4

Monosodium Iodoacetate (“MIA”)-Induced Osteoarthritis:

Male Wistar rats (175-200 g) were housed in solid bottom isolator cages, 2-4 rats per cage, with corncob bedding on a 12 hour:12 hour light:dark cycle. Animals were fed standard rat chow with water available ad libitum.

The rats were anesthetized with 5% volume/volume (“v/v”) isoflurane gas and maintained with 2% v/v isoflurane gas. The anesthetized rats were given a single intra-articular injection of 1 mg of MIA through the infrapatellar ligament of the right knee. MIA was dissolved in physiologic saline and administered in a volume of 50 μL. The contralateral control knee was injected with 50 μL of physiologic saline. Administration of isoflurane gas was discontinued, and the rats became fully conscious about 5 minutes later.

Shifts in hind paw weight distribution from the right to the left hind paws supporting the right (arthritic) and the left (contralateral control) hind leg knee joints were utilized as an index of joint pain and as a measure of compound efficacy. An incapacitance tester (Model 2KG, Linton Instrumentation, UK) was employed for determination of hind paw weight distribution. Each data point is the mean of three readings of 5 seconds duration.

CI-1027 (i.e., 6-(5-carboxy-5-methyl-hexyloxy)-2,2-dimethyl-hexanoic acid, calcium salt) was dissolved in hydroxypropylmethylcellulose (“HPMC”) vehicle (0.05% HPMC+0.2% Tween 80; compound amount was adjusted based on the percent of free acid, i.e., 6-(5-carboxy-5-methyl-hexyloxy)-2,2-dimethyl-hexanoic acid).

Two dosing paradigms were used to test the effect of CI-1027 in this model: (1) A single (acute) dose was utilized to determine the pain alleviating effects of CI-1027 on joint pain following acute, single dose administration; and (2) Chronic (multiple day; b.i.d.) dosing was utilized to determine the inhibitory effect of CI-1027 on joint pain and/or cartilage damage. The acute dosing paradigm relates to osteoarthritis signs such as mobility and joint function and osteoarthritis symptoms such as joint pain. The chronic dosing paradigm identifies disease modifying osteoarthritis drugs (“DMOADs”).

For the acute (single) dose paradigm, changes in hind paw weight distribution were determined late on Day 13 or early on Day 14 post MIA injection, as described previously, to establish a baseline pain reading. Rats were then given a single, 10, 30, or 100 mg/kg, dose of CI-1027 via oral gavage (PO). Changes in hind paw weight distribution were determined 2, 4 and 6 hours post-compound administration.

For the chronic (multiple) dosing paradigm, MIA and saline were injected on Day 0. CI-1027 was given PO 0.5 hour before MIA injection. CI-1027 (3, 10, or 30 mg/kg) was then given approximately every 12 hours for 28 days. Changes in hind paw weight distribution were determined on Days 7, 14, and 28.

After administering CI-1027 b.i.d. for 4 weeks, the rats were euthanized by CO₂ administration. The soft tissue was removed from the right (arthritic) leg, the joint was disarticulated, and the meniscus was carefully removed to expose the surface of the tibial plateau. The tibia was removed and placed in physiologic saline until erosion analysis was performed. Tibial plateau erosions were analyzed the same day as the sacrifice as follows. Each plateau was dipped in India ink for approximately 30 seconds to help define the erosions, rinsed in saline, and blotted on a paper towel. A stereomicroscope equipped with a digital camera was used to photograph each plateau. The tibial plateaus were photographed and graded separately by two ordinary skilled persons using the following system:

Grade 0=no erosion;

Grade I=erosion extending into the superficial or middle layers;

Grade II=deep layer erosion; no cartilage, sub-chondral bone exposed.

The photographs were transferred to a computer for image analysis using a Zeiss KS 300 Image Analysis System to determine the total erosion area for each grade, expressed in square millimeters (“mm²”). A RIDIT analysis (see above) was used to determine differences in overall erosion severity. This analysis takes into account both the erosion grade and area (small, medium and large, quantified by dividing the area of the largest erosion in each score into thirds) simultaneously. The analysis recognizes that each unit of severity is different, but does not assume a mathematical relationship between units.

Results for joint pain alleviation following acute administration:

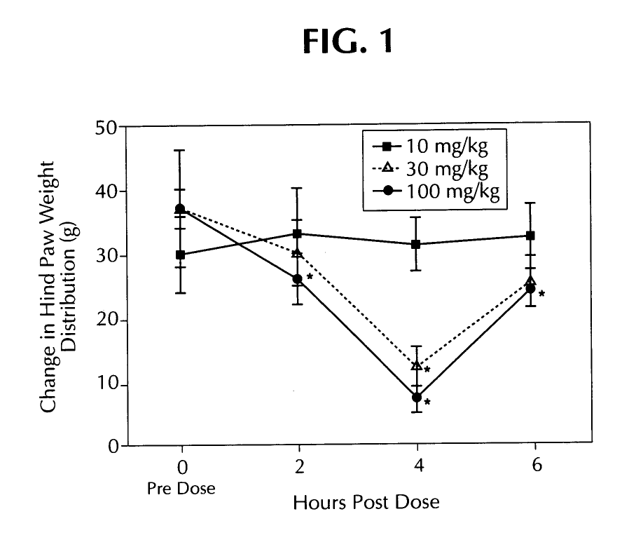

CI-1027 was tested in the rat MIA model in an acute dosing paradigm as described previously. MIA was injected into the right knee and saline into the left knee of all rats on Day 0. On Day 14 the rats were assessed on an incapacitance tester and then given CI-1027 (10, 30, or 100 mg/kg, PO). Two, four and six hours later, the rats were re-assessed. The results are graphically displayed in FIG. 1, which is a dose response line graph that shows the change in rat hind paw weight distribution, expressed in grams. In FIG. 1, administration of CI-1027 altered the shift in weight bearing potential (joint pain) in arthritic rats dosed with 100 mg/kg at 2, 4, and 6 hours post administration of CI-1027 in a statistically significant manner compared to pre-dose measurements. At the 30 mg/kg dose, only the change at the 4-hour post dose time point was statistically significant. Rats dosed with 10 mg/kg did not significantly alter their shift in weight bearing potential.

Statistically significant differences were determined by one-way ANOVA followed by Dunnett's multiple comparisons procedure. Data are expressed as mean±SEM. N=8 rats per group.

Results for joint pain alleviation following chronic administration of a single dose:

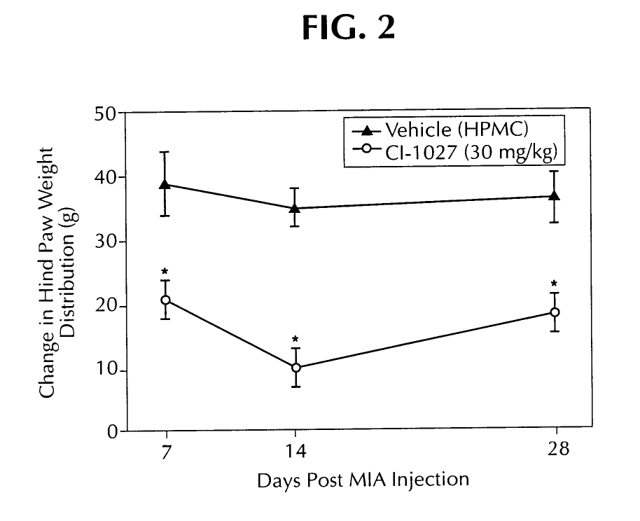

MIA and saline were injected on Day 0. CI-1027 (30 mg/kg) was given PO 0.5 hour before MIA injection. CI-1027 was then given b.i.d. for 28 days. Changes in hind paw weight distribution were determined on Days 7,14, and 28. The results are shown in FIG. 2, which is a time course line graph that shows the change in rat hind paw weight distribution, expressed in grams. As shown in FIG. 2, CI-1027 decreased the change in hind paw weight distribution at all three time-points tested in a statistically significant manner (47+8%, 71+9%, and 60±8% inhibition at Days 7, 14 and 28, respectively; p<0.05). Statistically significant differences were determined by One-Way ANOVA with the Dunnett's multiple comparisons procedure (*p<0.05). Data are expressed as mean±SEM. N=12 rats per group.

Results for joint pain alleviation following chronic administration for dose response:

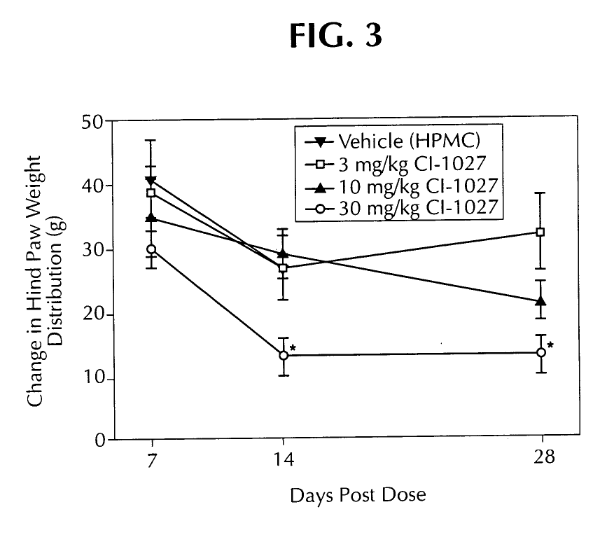

A dose response for C₁-11027 was also performed to look at joint pain as measured by changes in hind paw weight distribution after multiple doses. MIA and saline were injected on Day 0. CI-1027 (3, 10, or 30 mg/kg) was given PO 0.5 hour before MIA injection. CI-1027 was then given b.i.d. for 28 days. Changes in hind paw weight distribution were determined on Days 7, 14, and 28. The results are shown in FIG. 3, which is a time course, dose response line graph that shows the change in rat hind paw weight distribution, expressed in grams. As shown in FIG. 3, consistent with the previous experiment, CI-1027 at 30 mg/kg decreased the change in hind paw weight distribution at Days 14 and 28 in a statistically significant manner (50+10% and 62+8% inhibition, respectively; p<0.05). At Day 7, however, the significant effect observed in the first experiment was not repeated (38±14% inhibition). There were no statistically significant changes in hind paw weight distribution at the 10 and 3 mg/kg doses at all three time-points tested. Statistically significant differences were determined by One-Way ANOVA with the Dunnett's multiple comparisons procedure (*p<0.05). Data are expressed as mean±SEM. N=12 rats per group.

Results for inhibition of joint cartilage degradation following chronic administration:

CI-1027 was tested for the ability to preserve cartilage structure in the rat MIA model. Rats were dosed at 30 mg/kg, PO, b.i.d. for 28 days and sacrificed on Day 28. The tibial plateaus were removed and analyzed as previously described in the methods. MIA and saline were injected on Day 0. CI-1027 (30 mg/kg) was given PO 0.5 hour before MIA injection and was then given b.i.d. for 28 days. Erosion analysis was performed on Day 28. CI-1027 had no statistically significant effect on tibial plateau erosion severity when analyzed using the RIDIT test (p=0.10). N=12 rats per group. It should be appreciated that 2 of the 12 vehicle-treated rats had no erosions, which could partially account for the lack of statistical significance. Additionally, it should also be appreciated that 5 of the 12 rats treated with CI-1027 had no erosions.

The effect of CI-1027 on total erosion area (regardless of grade) was also determined. MIA and saline were injected on Day 0. CI-1027 (30 mg/kg) was given PO 0.5 hour before MIA injection and was then given b.i.d. for 28 days. Erosion analysis was performed on Day 28. There were no statistically significant differences CI-1027 decreased the total erosion area by 43%. However, this reduction was not statistically significant (as determined by t-test; p=0.179; N=12 rats per group), which again may be partially explained by the two vehicle rats with no erosions referenced above.

Results for inhibition of joint cartilage degradation following chronic administration for dose response:

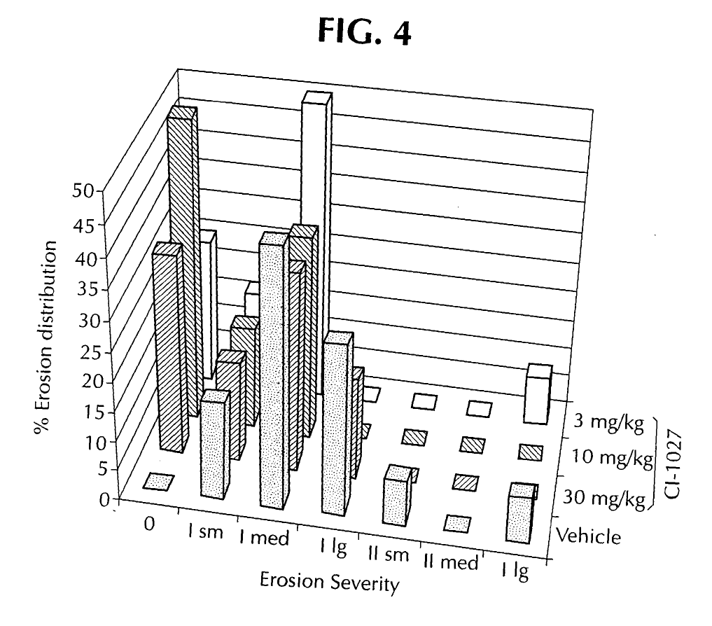

Rats were dosed with 3, 10 or 30 mg/kg, PO, b.i.d. for 28 days, tibial plateaus were removed and analyzed using the RIDIT test as described above. MIA and saline were injected on Day 0. CI-1027 (3, 10, or 30 mg/kg) was given PO 0.5 hour before MIA injection and was then given b.i.d. for 28 days. Erosion analysis was performed on Day 28. All three doses were statistically significant as determined by RIDIT analysis (p<0.05); N=12 rats per group. The results are shown in FIG. 4, which is a dose response bar graph that shows effects on (i) cartilage erosion severity, expressed as Grade 0 (no erosion), Grade I (erosion extending into the superficial or middle cartilage layers and further characterized by size small, medium, and large), or Grade II (deep layer erosion; no cartilage remaining in spots, subchondral bone exposed in spots, and further characterized by size small, medium, and large) and (ii) cartilage size, expressed as percent erosion distribution, relative to vehicle-injected control animals. As shown in FIG. 4, all three doses of CI-1027 had a statistically significant inhibitory effect on erosion severity. It should be appreciated that all vehicle-treated rats had erosions, as opposed to the previously described experiment.

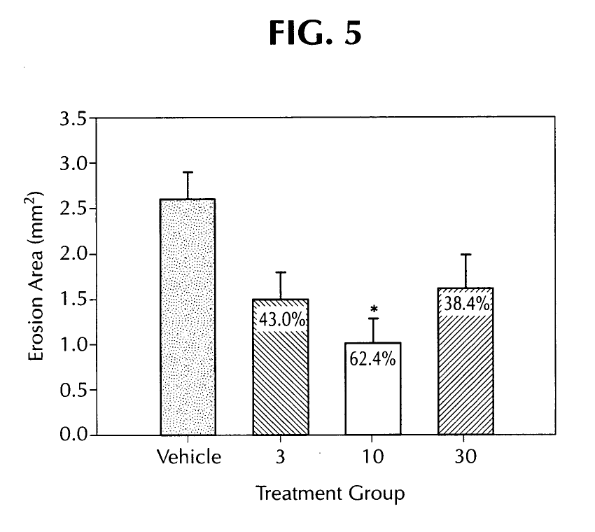

The effect of CI-1027 (3, 10, and 30 mg/kg) on total erosion area (regardless of grade) was also determined. MIA and saline were injected on Day 0. CI-1027 (3, 10, or 30 mg/kg) was given PO 0.5 hour before MIA injection and was then given b.i.d. for 28 days. Erosion analysis was performed on Day 28. Statistical significance was determined by One-Way ANOVA, Dunnett's multiple comparisons procedure (p<0.05; N=12 rats per group). The results are shown in FIG. 5, which is a dose response bar graph that shows effects on total cartilage erosion area, expressed in square millimeters, regardless of cartilage erosion severity grade, relative to vehicle-injected control animals. As shown in FIG. 5, a statistically significant decrease in total erosion area was noted only at the 10 mg/kg dose of CI-1027 (62.4%).

A single dose of CI-1027 at 30 or 100 mg/kg effectively alleviated joint pain in MIA-induced arthritic rats, demonstrating that CI-1027 and the other active compounds of this invention method have a direct antihyperalgesic effect on joint pain. CI-1027 also improved joint pain in the rat MIA model when administered chronically. CI-1027 administered at 30 mg/kg, PO, b.i.d. improved joint pain after 1-2 weeks of dosing with sustained effects out to 4 weeks. In addition, CI-1027 effectively decreased medial tibial plateau erosion size when administered at 3, 10 or 30 mg/kg, PO, b.i.d. for 28 days, demonstrating that CI-1027 and the other active compounds of this invention method have a direct cartilage damage inhibiting effect in a joint undergoing cartilage damage.

### Biological Method 5

Interleukin-6 (“IL-6”) with Interleukin-6 Soluble Receptor (“IL-6sR”) Induced Joint Pain:

The IL-6/IL-6sR model relates to osteoarthritis pain.

Male Wistar rats (175-200 g) were housed in solid bottom isolator cages, 2-4 rats per cage, with corncob bedding on a 12 hour: 12 hour light:dark cycle. Animals were fed standard rat chow with water available ad libitum.

Human recombinant IL-6 (100 ng per rat; R&D Systems, Minneapolis, Minn.) was incubated at room temperature with human recombinant IL-6soluble receptor (300 ng per rat; R&D Systems, Minneapolis, Minn.) in phosphate buffered saline (“PBS”) for 15 minutes prior to injection. Rats were anesthetized with 5% volume/volume (“v/v”) isoflurane gas and maintained with 2% v/v isoflurane gas. The anesthetized rats were given a single intra-articular injection of IL-6 μL-6sR through the infrapatellar ligament of the right knee in a volume of 50 μL. The contralateral control knee was injected with 50 μL of PBS. Administration of isoflurane gas was discontinued, and the rats became fully conscious about 5 minutes later.

Shifts in hind paw weight distribution from the right (arthritic) to the left (contralateral control) paws were utilized as an index of joint pain and as a measure of compound efficacy. An incapacitance tester (Model 2KG, Linton Instrumentation, UK) was employed for determination of hind paw weight distribution. Each data point is the mean of three, 5-second readings.

CI-1027 was dissolved in HPMC vehicle as described above. Baseline changes in hind paw weight distribution readings were determined one day prior to injection of IL-6/IL-6sR. On the day of the experiment (Day 0), a single dose of CI-1027 (10, 30 or 100 mg/kg) was given via oral gavage 3 hours prior to injection of IL-6/IL-6sR (n=10 rats per group). Changes in hind paw weight distribution were determined one, three, and six hours after injection of IL-6/IL-6sR.

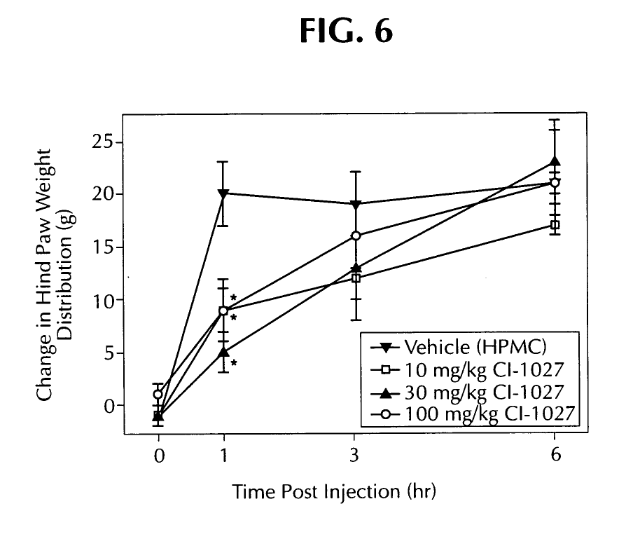

The effect of CI-1027 (10, 30, and 100 mg/kg) on joint pain was tested in the rat IL-6/IL-6sR model. Baseline incapacitance readings were taken on Day-1. IL-6 μL-6sR and PBS were injected on Day 0. CI-1027 (10, 30, or 100 mg/kg) was given PO 3 hours before IL-6/IL-6sR injection. Changes in hind paw weight distribution were determined 1, 3, and 6 hours post injection (4, 6, and 9 hours post CI-1027 dose). The results are shown in FIG. 6, which is a time course, dose response line graph that shows the change in rat hind paw weight distribution, expressed in grams. As shown in FIG. 6, all three doses of CI-1027 decreased the change in hind paw weight distribution at 1 hour post injection, 4hours post CI-1027 administration in a statistically significant manner (53±9%, 74±8%, and 57±15% inhibition at 10, 30 and 100 mg/kg, respectively. Statistically significant differences were determined by One-Way ANCOVA followed by the Hochberg's procedure (*p<0.001). Data are expressed as mean±SEM. N=10 rats per group. No significant effect was noted at 3 and 6 hours post injection (6, and 9 hours post CI-1027 administration).

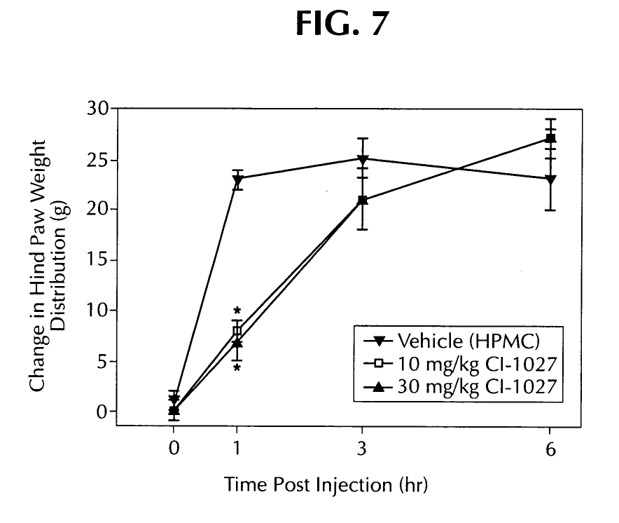

The experiment described above was repeated with the following changes: CI-1027 was tested only at 10 and 30 mg/kg and all incapacitance test readings were blinded. Baseline incapacitance readings were taken on Day-1. IL-6/IL-6sR and PBS were injected on Day 0. CI-1027 (10 or 30 mg/kg) was given PO 3 hours before IL-6/IL-6sR injection. Changes in hind paw weight distribution were determined 1, 3, and 6 hours post injection (4, 6, and 9 hours post CI-1027 dose). Statistically significant differences were determined by One-Way ANCOVA followed by the Hochberg's procedure (*p<0.001). Data are expressed as mean 1SEM. N=10 rats per group. The results are shown in FIG. 7, which is a time course, dose response line graph that shows the change in rat hind paw weight distribution, expressed in grams. As shown in FIG. 7 and as previously seen in the first experiment, both the 10 and the 30 mg/kg doses of CI-1027 decreased change in hind paw weight distribution as compared to the vehicle group in a statistically significant manner (63±6% and 71±10% inhibition at 10 and 30 mg/kg, respectively).

In the rat IL-6/IL-6sR knee injection model, CI-1027 significantly alleviated joint pain 1-hour post injection of the IL-6/IL-6sR, demonstrating that CI-1027 and the other active compounds of this invention method have a direct antihyperalgesic effect on joint pain.

### Biological Method 6

Carrageenan-Induced Thermal Hyperalgesia:

Purpose: to screen compounds for potential hyperalgesia-based analgesic efficacy (i.e., alleviation of inflammatory pain such as rheumatoid arthritic pain).

Male Sprague Dawley rats (200-300 g), obtained from Charles River, were housed in groups of 2 under a 12 hour light/dark cycle with food and water ad libitum. Animals were allowed to acclimate to the test room one hour prior to testing.

Thermal hyperalgesia was assessed using the rat plantar test (apparatus from Univ. California, San Diego) following a modified method of Hargreaves et al., 1988. Rats were habituated to the apparatus, which consisted of three perspex boxes that individually housed 2 rats per box on an elevated glass table. A mobile radiant heat source (halogen lamp) was located under the glass table and focused onto the hind paw. Paw withdrawal latencies (PWL) were recorded in seconds (“s”). An automatic cut off point of 22.5 s was set to prevent tissue damage. The mean of 2-3 PWL was taken at each time point for both hind paws of each animal. The apparatus was calibrated to give a PWL of approximately 10 s before carrageenan administration.

After basal PWL were determined, animals receive an intraplantar 100 μL of 10 mg/mL) into the right hind paw. Lambda carrageenan (Sigma Chemical Co.) was dissolved in isotonic saline 5 minutes prior to injecting. PWL were reassessed following the same protocol 2 hours post carrageenan (this time point represented the start of peak hyperalgesia) to ascertain that hyperalgesia had developed. 6-(5-Carboxy-5-methyl-hexyloxy)-2,2-dimethyl-hexanoic acid, calcium salt was then administered orally via oral dosing needles 2.5 hours post carrageenan injection, and PWL were taken again at 30- or 60-minute intervals post drug administration for 6 hours.

Data consisted of the mean of 2-3 reads/paw for individual animals. Group data was the compiled individual means. Percent inhibition was calculated as follows for each time point: (mean drug treated−mean control)/(mean baseline−mean control)×100.

The inhibition results were then statistically analyzed.

Results for use of CI-1027 for alleviating joint pain expressed as percent inhibition of a change in hind paw weight distribution (MIA and IL-6) or paw withdrawal latency (CITH) are shown below in Table 2. Table 2. Alleviation of joint pain as measured by inhibition of a change in hind paw weight distribution (MIA and IL-6) or paw withdrawal latency (CITH) by one single oral dose of 30 mg/kg of 6-(5-carboxy-5-methyl-hexyloxy)-2,2-dimethyl-hexanoic acid, calcium salt on Day 7 (MIA) or Day 1 (1L-6 and CITH) measured at 4 hours post dose:

| | Inhibition of |
|---|---|
| | Pain (% versus |
| Model | control) |
| MIA (acute) | 67 ± 8%* |
| IL-6 | 71 ± 10%* |
| CITH | 65 ± 20%* |

*ρ < 0.05.

The following substituted dialkyl ether compounds (reference numbers) and substituted alkyl compound (reference number) have been assayed for pain alleviation effects in at least one of the above methods selected from MIA, IL-6/IL-6sR, and CITH:

7,7′-oxybis(2,2-dimethylheptanoic acid) (Al);

5,5′-oxybis(2,2-dimethylpentanoic acid) (A2);

4,4′-oxybis(2,2-dimethylbutanoic acid) (A3); and

2,2,12,12-tetramethyltridecanedioic acid (A4).

The data are shown below in Tables 3 to 5. In Table 3 below, data from the method of Biological Method 4 are expressed as a percent inhibition of a change in hind paw weight distribution±the standard error of the mean. The percents inhibition were calculated according to the formula recited above in Biological Method 1.

Table 3. Following the method of Biological Method 4, alleviation of joint pain as measured by inhibition of a MIA-induced change in hind paw weight distribution by one single oral dose of 30 mg/kg of compound reference numbers A1 to A4 on Day 7 or Day 14 measured at 2, 4, or 6 hours post dose of compound, expressed as a percentage inhibition±the standard error of the mean, as shown below in the columns headed “Cpd. Ref. No.,” “Day 7 2 hr. (%±SEM),”“Day 7 4 hr. (%±SEM),”“Day 7 6 hr. (%±SEM),”“Day 14 2 hr. (%±SEM),”“Day 14 4 hr. (%±SEM),” and “Day 14 6 hr. (%±SEM),”, respectively:

| Cpd. | Day 7 | Day 7 | Day 7 | Day 14 | Day 14 | Day 14 |
|---|---|---|---|---|---|---|
| Ref. | 2 hr. | 4 hr. | 6 hr. | 2 hr. | 4 hr. | 6 hr. |
| No. | (% ± SEM) | (% ± SEM) | (% ± SEM) | (% ± SEM) | (% ± SEM) | (% ± SEM) |
| A1 | 50 ± 11 | 15 ± 14 | N/d¹ | −3 ± 15 | −9 ± 17 | N/d |
| A2 | −11 ± 15 | 16 ± 15 | N/d | 9 ± 13 | 9 ± 20 | N/d |
| A3 | 20 ± 4 | 34 ± 5 | 81 ± 6 | 33 ± 7 | 36 ± 3 | 27 ± 9 |
| A4 | 25 ± 5 | 38 ± 7 | 43 ± 6 | 35 ± 6 | 29 ± 8 | 55 ± 7 |

N/d means no data available

In Table 4 below, data from the method of Biological Method 5 are expressed as a percent inhibition of a change in hind paw weight distribution±the standard error of the mean. The percents inhibition were calculated as follows. Percent inhibition of a change in hind paw weight distribution

$$\begin{matrix}Percent inhibition of a change in \\ hind paw weight distribution\end{matrix} = \{ 1 - [ \frac{( Δ {W}_{G} )}{( Δ {W}_{C} )} ] \} \times 100$$

wherein:

ΔW_{C} is the hind-paw weight differential between the healthy limb and the hrIL-6+hrIL-6sR injected limb of the control animal group administered vehicle alone, as measured at time 1, 3, or 6 hours post injection; and

ΔW_{G} is the hind-paw weight differential between the healthy limb and the hrIL-6+hrIL-6sR injected limb of a treatment animal group as measured at time 1, 3, or 6 hours post injection.

Table 4. Following the method of Biological Method 5, alleviation of joint pain as measured by inhibition of a human recombinant (IL-6IL-6sR)-induced change in hind paw weight distribution by one single oral dose of 30 mg/kg of compound reference number A4 on Day 1 measured at 1, 3, or 6 hours post injection of the IL-6/IL-6sR, expressed as a percentage inhibition±the standard error of the mean, as shown below in the columns headed “Cpd. Ref. No.,” “1 hr. (%±SEM),”“3 hr. (%±SEM),” and “6 hr. (%±SEM),”, respectively:

| Cpd. | | | |
|---|---|---|---|
| Ref. | 1 hr. | 3 hr. | 6 hr. |
| No. | (% ± SEM) | (% ± SEM) | (% ± SEM) |
| A4 | 70 ± 9 | 79 ± 5 | 26 ± 6 |

Table 5. Following the method of Biological Method 6, alleviation of pain as measured by paw withdrawal latency in CITH by one single oral dose of 30 mg/kg or 100 mg/kg of compound reference numbers A1 to A4 on Day 1, expressed as a percentage inhibition, as shown below in the columns headed “Cpd. Ref. No.,” “Dose (mg/kg),” and “CITH (%),”, respectively:

| Cpd. Ref. | Dose | CITH |
|---|---|---|
| No. | (mg/kg) | (%) |
| A1 | 30 | 22 |
| A2 | 30 | −3 |
| A3 | 30 | 20 |
| A4 | 30 | 118 |
| A4 | 100 | 115 |

### Biological Method 7

The method according to Biological Method 5, wherein the 1L-6, IL-6sR, or IL-6 and IL-6sR is replaced by a protein or protein and its receptor, respectively, selected from: oncostatin-M, oncostatin-M and oncostatin-M receptor, leukemia inhibitor factor (“LIF”), LIF and leukemia inhibitor factor receptor (“LIFR”), interleukin-11 (“IL-11”), and IL-11 and interleukin-11 receptor (“IL-11R”).

A method of assaying activity of a substituted dialkyl ether, substituted aryl-alkyl ether, substituted dialkyl thioether, substituted dialkyl ketone, or substituted-alkyl compound in rheumatoid arthritis is described below in

### Biological Method 8

Monoclonal Antibody-Induced Arthritis (“MAIA”) Mouse Model:

Rapid induction of arthritis in mice in this model is made possible through the utilization of a commercially available arthritis-inducing monoclonal antibody cocktail. This cocktail contains four different monoclonal antibodies (“mAbs”) that were generated in DBA/1 (H-2^{q} haplotype) mice with active collagen-induced arthritis (“CIA;” CIA may be induced in mice by intradermal immunization of susceptible animals with native bovine type II collagen in complete Freund's adjuvant, but this typically takes 6-8 weeks and the incidence and severity of disease can be quite variable, requiring relatively large numbers of mice to achieve statistical significance). Three of the mAbs recognize autoantigenic epitopes clustered within an 84 amino acid residue fragment, LyC2 (smallest arthrogenic fragment of type II collagen) of CB 11 and the fourth mAb reacts with LyC2 (Terato, K., Harper, D., Griffiths, M., Hasty, D., Ye, J., Cremer, M. and J, Seyer. Collagen-induced arthritis in mice: synergistic effect of E. coli lipopolysaccharide bypasses epitopespecificity in the induction of arthritis with monoclonal antibodies to type II collagen. Autoimmunity. 22(3):137-147). More importantly, all four mAbs recognize epitopes of type II collagen that are conserved among many animal species and thus crossreact with both homologous and heterologous type II collagen.

One of the main benefits of the monoclonal antibody-induced arthritis protocol over a traditional CIA model is its ability to induce a CIA-like arthritis in strains of mice other than DBA/1. As most knockout mice are made in 129/SvJ, BALB/c or C57BL/6 strains, and not DBA/1, it has been difficult to use knockouts to validate targets for rheumatoid arthritis. Researchers were forced to spend years backcrossing their knockout mice onto the DBA/1 background in order to test the effect of a genetic deficiency on the onset or severity of arthritis.

When the 129/SvJ, BALB/c, C3H/FeJ, and C57BL/6 strains were tested, DBA/1 was found to be the most responsive, but BALB/c, C57BL/6, and 129/SvJ strains were only slightly less responsive than the DBA/1. The C3H/HeJ mice were relatively unresponsive. The response of the 129/SvJ mice to the combination of mAb and LPS was also low, but still statistically significantly higher than in 129/SvJ mice injected with LPS only.

One of the hallmarks of a good preclinical disease model is that agents that work to alleviate the human disease also work in the animal model. Methotrexate, cyclosporin A, and anti-TNF-α antibodies have all been used in the successful treatment of rheumatoid arthritis in a human. When tested in BALB/c mice using the monoclonal antibody-induced arthritis model, methotrexate (4 mg/kg) and anti-TNF-α antibody (83.33 μg) were both found to significantly inhibit footpad and ankle swelling throughout the course of the disease. Anti-TNF-α antibody (83.33 μg/animal) was administered i.p. one and five days after injection of Arthrogen-CIA antibody. Cyclosporin A (25 mg/kg) demonstrated an early inhibition of the swelling response, but by Day 9 there was no difference from the vehicle control.

MAIA Protocol:

In a typical MAIA experiment, groups of four mice were injected intraperitoneally (“i.p.”) with 0.4 mL (BALB/c and DBA/1 strains) or 0.8 mL (C57BL/6, 129X1/SvJ, C3H/FeJ, and C3H/FeJ strains) with a 10 mg/mL solution of Arthrogen-CIA Monoclonal Antibody Blend (Chemicon International, Inc.). Two days later, the mice were injected i.p. with 0.2 mL of a 0.25 mg/mL solution of LPS (lipopolysaccharide form Escherichia coli (“E. coli ”) strain 0111B4) in PBS. Footpad and ankle size was assessed using a Dyer Digital Caliper (#655-030-4916) on the day of mAb injection, and every two days following LPS injection.

In experiments involving CI-1027, 30, 100, or 200 mg/kg of 6-(5-carboxy-5-methyl-hexyloxy)-2,2-dimethyl-hexanoic acid, calcium salt were dosed orally 1 day prior to injection of the Arthrogen-CIA antibody and then daily thereafter.

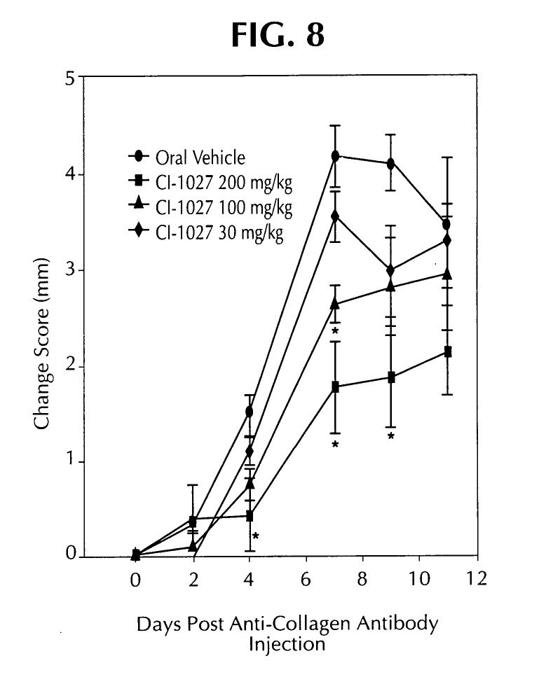

The results for oral administration of vehicle or 30, 100, or 200 mg/kg of 6-(5-carboxy-5-methyl-hexyloxy)-2,2-dimethyl-hexanoic acid, calcium salt, are shown in FIG. 8. In FIG. 8, a time course, dose response line graph shows that the compound 6-(5-carboxy-5-methyl-hexyloxy)-2,2-dimethyl-hexanoic acid, calcium salt when dosed at 200 mg/kg inhibits a change in mouse ankle joint and paw swelling, expressed in millimeters (“mm”), in a statistically significant manner (p<0.05) as measured on Days 4, 7, and 9 and at 100 mg/kg on Day 7.

### Biological Method 9

Pain-Alleviating Effect of a Combination of a Substituted Dialkyl Ether and a COX-2Inhibitor in MIA:

Results for Joint Pain Alleviation Following Acute Administration:

6-(5-carboxy-5-methyl-hexyloxy)-2,2-dimethyl-hexanoic acid, calcium salt (“CI-1027”) and rofecoxib was tested in the rat MIA model in an acute dosing paradigm as described previously. MIA was injected into the right knee and saline into the left knee of all rats on Day 0. On Day 14 the rats were assessed on an incapacitance tester and then given 6-(5-carboxy-5-methyl-hexyloxy)-2,2-dimethyl-hexanoic acid, calcium salt (10 mg/kg, PO) and rofecoxib (3 mg/kg, PO). Two hours later, the rats were reassessed. The results for inhibition of pain, expressed as a percentage±standard error of the mean, are shown below in Table 5. The percent inhibition of pain was determined as described above in Biological Method 1. In Table 5, administration of 6-(5-carboxy-5-methyl-hexyloxy)-2,2-dimethyl-hexanoic acid, calcium salt and rofecoxib altered the shift in weight bearing potential (joint pain) in arthritic rats at 2 hours post administration of 6-(5-carboxy-5-methyl-hexyloxy)-2,2-dimethyl-hexanoic acid, calcium salt and rofecoxib in a statistically significant manner compared to pre-dose measurements as well as compared to 6-(5-carboxy-5-methyl-hexyloxy)-2,2-dimethyl-hexanoic acid, calcium salt or rofecoxib alone. Statistically significant differences were determined by one-way ANOVA followed by Dunnett's multiple comparisons procedure and indicated in Table 5 with a “*.” Data are expressed as mean %±SEM. N=8 rats per group.

TABLE 5

| | | | CI-1027 (10 |
|---|---|---|---|
| | | | mg/kg) + |
| | CI-1027 | Rofecoxib | rofecoxib |
| | (10 mg/kg) | (3 mg/kg) | (3 mg/kg) |
| Mean pain | 20 ± 9 | 23 ± 7 | 36 ± 11* |
| alleviation at 2 hours | | | |
| post administration | | | |
| (% ± SEM) | | | |

*P < 0.05

The foregoing studies establish, or would establish, that substituted dialkyl ether, substituted aryl-alkyl ether, substituted dialkyl thioether, substituted dialkyl ketone, or substituted-alkyl compounds useful in the invention method, or a pharmaceutically acceptable salt thereof, including a compound named 6-(5-carboxy-5-methyl-hexyloxy)-2,2-dimethyl-hexanoic acid, calcium salt, or a combination comprising such a compound and a COX-2 inhibitor, are effective for the prevention and inhibition of cartilage damage, improvement of joint function, alleviation of pain, including IL-6 induced joint pain, mechanical pain, OA pain, inflammatory pain, RA pain, acute pain, chronic pain, alleviation of acute pain, alleviation of chronic pain, and the like, and prevention and treatment of osteoarthritis and rheumatoid arthritis in human, and other mammalian patients. Such a treatment offers a distinct advantage over existing treatments that only modify pain and other secondary symptoms. The effectiveness of a substituted dialkyl ether, substituted aryl-alkyl ether, substituted dialkyl thioether, substituted dialkyl ketone, or substituted-alkyl compound useful in the invention method, or a pharmaceutically acceptable salt thereof, including a compound named 6-(5-carboxy-5-methyl-hexyloxy)-2,2-dimethyl-hexanoic acid, calcium salt, in the MIA model indicate that substituted dialkyl ether, substituted aryl-alkyl ether, substituted dialkyl thioether, substituted dialkyl ketone, or substituted-alkyl compounds useful in the invention method, or a pharmaceutically acceptable salt thereof, including a compound named 6-(5-carboxy-5-methyl-hexyloxy)-2,2-dimethyl-hexanoic acid, calcium salt, will have clinically useful effects in preventing and/or treating cartilage damage, improving joint function, and alleviating pain.

Administration according to the invention method of an active compound useful in the invention method, or a pharmaceutically acceptable salt thereof, to a mammal to treat the diseases listed above may be, but is not necessarily, accomplished by administering the compound, or a salt thereof, in a pharmaceutical dosage form.

The substituted dialkyl ether, substituted aryl-alkyl ether, substituted dialkyl thioether, substituted dialkyl ketone, or substituted-alkyl compounds useful in the invention method, or a pharmaceutically acceptable salt thereof, can be prepared and administered according to the invention method in a wide variety of oral and parenteral pharmaceutical dosage forms. Thus, the substituted dialkyl ether, substituted aryl-alkyl ether, substituted dialkyl thioether, substituted dialkyl ketone, or substituted-alkyl compounds useful in the invention method, or a pharmaceutically acceptable salt thereof, can be administered by injection, that is, intravenously, intramuscularly, intracutaneously, subcutaneously, intraduodenally, or intraperitoneally. Also, the substituted dialkyl ether, substituted aryl-alkyl ether, substituted dialkyl thioether, substituted dialkyl ketone, or substituted-alkyl compounds useful in the invention method, or a pharmaceutically acceptable salt thereof, can be administered by inhalation, for example, intranasally. Additionally, the substituted dialkyl ether, substituted aryl-alkyl ether, substituted dialkyl thioether, substituted dialkyl ketone, or substituted-alkyl compounds useful in the invention method, or a pharmaceutically acceptable salt thereof, can be administered transdermally. It will be obvious to those skilled in the art that the following dosage forms may comprise as the active components either an active compound useful in the invention method, or a pharmaceutically acceptable salt thereof. The active compounds generally are present in a concentration of about 5% to about 95% by weight of the formulation.

For preparing pharmaceutical compositions from the substituted dialkyl ether, substituted aryl-alkyl ether, substituted dialkyl thioether, substituted dialkyl ketone, or substituted-alkyl compounds useful in the invention method, or a pharmaceutically acceptable salt thereof, (i.e., the active components) pharmaceutically acceptable carriers can be either solid or liquid. Solid form preparations include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or an encapsulating material.

In powders, the carrier is a finely divided solid that is in a mixture with the finely divided active component. Powders suitable for intravenous administration or administration by injection may be lyophilized.

In tablets, the active component is mixed with the carrier having the necessary binding properties in suitable proportions and compacted in the shape and size desired.

The powders and tablets typically contain from about 5% to about 70%, total, of the active component. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active component with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation may optionally be in unit dosage form. In such form, the preparation is subdivided into unit doses having an appropriate quantity of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.01 to 1000 mg, including from 1 to 500 mg or from 10 to 250 mg, according to the particular application and the potency of the active components. The composition can, if desired, also contain other compatible therapeutic agents.

In therapeutic use as agents to treat the above-listed diseases, the substituted dialkyl ether, substituted aryl-alkyl ether, substituted dialkyl thioether, substituted dialkyl ketone, or substituted-alkyl compounds useful in the invention method, or a pharmaceutically acceptable salt thereof, or a combination of the same with another therapeutic agent, are administered at a dose that is effective for improving at least one clinical measure, pathological hallmark, or symptom of the disease or disorder being treated. The initial dosage of about 1 mg/kg to about 100 mg/kg daily of the active component will be effective. A daily dose range of about 25 mg/kg to about 75 mg/kg of the active component is typical. The dosages, however, may be varied depending upon the requirements of the patient, the severity of the condition being treated, and the particular substituted dialkyl ether, substituted aryl-alkyl ether, substituted dialkyl thioether, substituted dialkyl ketone, or substituted-alkyl compound useful in the invention method, or a pharmaceutically acceptable salt thereof, or combination being employed. Determination of the proper dosage for a particular situation is within the skill of the art as described above. Typical dosages will be from about 0.1 mg/kg to about 500 mg/kg, and ideally about 25 mg/kg to about 250 mg/kg, such that it will be an amount that is effective to treat the particular disease or disorder being treated.

A typical composition for dogs comprises an ingestible liquid peroral dosage form selected from the group consisting of a solution, suspension, emulsion, inverse emulsion, elixir, extract, tincture and concentrate, optionally to be added to the drinking water of the dog being treated. Any of these liquid dosage forms, when formulated in accordance with methods well known in the art, can either be administered directly to the dog being treated, or may be added to the drinking water of the dog being treated. The concentrate liquid form, on the other hand, is formulated to be added first to a given amount of water, from which an aliquot amount may be withdrawn for administration directly to the dog or addition to the drinking water of the dog.

A composition can provide delayed-, sustained- and/or controlled-release of an active compound useful in the invention method, or a pharmaceutically acceptable salt thereof. Such compositions include all such dosage forms which produce≧40% inhibition of cartilage degradation or pain, and result in a plasma concentration of the active component of, for example, at least 3 fold the active component's ED₄₀ for at least 2 hours; for at least 4 hours; for at least 8 hours; for at least 12 hours; for at least 16 hours; for at least 20 hours; or for at least 24 hours. For example, there is included within the above-described dosage forms those which produce≧40% inhibition of cartilage degradation or pain, and result in a plasma concentration of the active component of at least 5 fold the active component's ED₄₀ for at least 2 hours, for at least 2 hours, for at least 8 hours, for at least 12 hours, for at least 20 hours and for at least 24 hours. Further, there is included the above-described dosage forms which produce >50% inhibition of cartilage degradation or pain, and result in a plasma concentration of the active component of at least 5 fold the active component's ED₄₀ for at least 2 hours, for at least 4 hours, for at least 8 hours, for at least 12 hours, for at least 20 hours and for at least 24 hours.

The above formulation examples illustrate the invention pharmaceutical compositions having an effective amount of an active compound, an invention combination, or compositions having discrete formulations of the active components of the invention combination and a pharmaceutically acceptable carrier, diluent, or excipient and a pharmaceutically acceptable carrier, diluent, or excipient. The examples are representative only, and are not to be construed as limiting the invention in any respect.

While it may be desirable to formulate another therapeutic agent and 6-(5-carboxy-5-methyl-hexyloxy)-2,2-dimethyl-hexanoic acid, calcium salt, or a pharmaceutically acceptable salt thereof, together in one capsule, tablet, ampoule, solution, and the like, for simultaneous administration, it is not necessary for the purposes of practicing the invention methods. For example, valdecoxib and a 6-(5-carboxy-5-methyl-hexyloxy)-2,2-dimethyl-hexanoic acid, calcium salt, or a pharmaceutically acceptable salt thereof, of an invention combination alternatively can each be formulated independently in any form such as, those of any one Formulation Examples 1 to 16, and administered either simultaneously or at different times.

It should be appreciated that the invention methods comprising administering an invention combination to a mammal to treat diseases or disorders listed above may be used to treat different diseases simultaneously. For example, administration of valdecoxib in accordance with the invention combination may be carried out as described above to treat inflammation, arthritic pain, pain associated with menstrual cramping, and migraines, while an active compound useful in the invention method, or a pharmaceutically acceptable salt thereof, may be administered to treat osteoarthritis, rheumatoid arthritis, improve joint function, alleviate pain, or inhibit cartilage damage.

As shown above, the invention method offers a distinct advantage over existing treatments for diseases such as osteoarthritis that comprise cartilage damage, wherein the existing treatments modify pain or secondary symptoms, but do not show a disease modifying effect. Substituted dialkyl ether, substituted aryl-alkyl ether, substituted dialkyl thioether, substituted dialkyl ketone, or substituted-alkyl compounds useful in the invention method, or a pharmaceutically acceptable salt thereof, including a compound named 6-(5-carboxy-5-methyl-hexyloxy)-2,2-dimethyl-hexanoic acid, calcium salt, have been shown to be useful for inhibiting cartilage damage, preventing or treating rheumatoid arthritis, improving joint function, treating osteoarthritis or alleviating pain.

While the invention has been described and illustrated above with reference to certain particular aspects and embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

All patents, patent applications, and publications, including patent application publications, cited herein are hereby incorporated by reference in their entirety for all purposes.

Having described invention methods, compositions, and combinations, various aspects and embodiments of the invention are hereupon initially claimed.

What is claimed is:

1. A method of treating a disease or disorder selected from arthritis, cartilage damage, joint pain, joint inflammation, systemic lupus erythematous, mixed connective tissue disease, and sepsis in a mammal, comprising administering to the mammal a therapeutically effective amount of a substituted dialkyl ether of Formula I 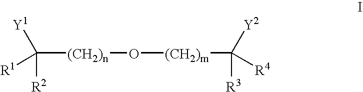
or a pharmaceutically acceptable salt thereof,
wherein:
n and m independently are integers of from 2 to 9;
R¹, R², R³, and R⁴ independently are C₁-C₆ alkyl, C₂-C₆ alkenyl, or C₂-C₆alkynyl; or
R¹ and R² together with the carbon atom to which they are attached, or R³ and R⁴ together with the carbon atom to which they are attached, or R¹ and R² together with the carbon atom to which they are attached and R³ and R⁴ together with the carbon atom to which they are attached, can complete a carbocyclic ring having from 3 to 6 carbons;
Y¹ and Y² independently are COOH, CHO, tetrazole, or COOR⁵, wherein R⁵ is C₁-C₆ alkyl, C₂-C₆ alkenyl, or C₂-C₆ alkynyl; and
wherein the alkyl, alkenyl, and alkynyl groups may be substituted with one or two groups selected from halo, hydroxy, C₁-C₆ alkoxy, and phenyl.

2. The method according to claim 1, wherein the arthritis is osteoarthritis.

3. The method according to claim 1, wherein the arthritis is rheumatoid arthritis.

4. The method according to claim 1, wherein the arthritis is selected from gouty arthritis, juvenile arthritis, psoriatic arthritis, and ankylosing spondylitis.

5. The method according to claim 1, wherein the joint pain is osteoarthritic joint pain.

6. The method according to claim 1, wherein the joint pain is rheumatoid arthritic joint pain.

7. The method according to claim 1, wherein the joint pain is inflammatory joint pain.

8. The method according to claim 1, wherein the joint pain is acute joint pain.

9. The method according to claim 1, wherein the joint pain is chronic joint pain.

10. The method according to claim 1, wherein the joint inflammation is arthritic joint inflammation.

11. The method according to claim 1, wherein the joint inflammation is rheumatoid arthritic joint inflammation.

12. The method according to claim 1, wherein the cartilage damage is in an osteoarthritic joint.

13. The method according to claim 1, wherein the cartilage damage is in an rheumatoid arthritic joint.

14. The method according to any one of claims 1 to 13, wherein the substituted dialkyl ether is a compound named 6-(5-carboxy-5-methyl-hexyloxy)-2,2-dimethyl-hexanoic acid, or a pharmaceutically acceptable salt thereof.

15. The method according to claim 14, wherein the 6-(5-carboxy-5-methyl-hexyloxy)-2,2-dimethyl-hexanoic acid, or a pharmaceutically acceptable salt thereof, is selected from:
6-(5-carboxy-5-methyl-hexyloxy)-2,2-dimethyl-hexanoic acid, calcium salt hydrate;
Crystal Form 1 of 6-(5-carboxy-5-methyl-hexyloxy)-2,2-dimethyl-hexanoic acid, calcium salt; and
Crystal Form 2 of 6-(5-carboxy-5-methyl-hexyloxy)-2,2-dimethyl-hexanoic acid, calcium salt.

16. The method according to claim 14, wherein the 6-(5-carboxy-5-methyl-hexyloxy)-2,2-dimethyl-hexanoic acid, or a pharmaceutically acceptable salt thereof, is a compound named 6-(5-carboxy-5-methyl-hexyloxy)-2,2-dimethyl-hexanoic acid, calcium salt.

17. A method of treating a disease or disorder selected from arthritis, cartilage damage, joint pain, joint inflammation, systemic lupus erythematous, mixed connective tissue disease, and sepsis in a mammal, comprising administering to the mammal a therapeutically effective amount of a substituted-alkyl compound of Formula II 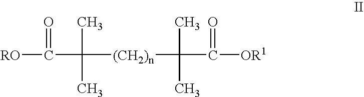
or a pharmaceutically acceptable salt thereof,
wherein n is 6, 7, 8, 9, or 10; and
R and R¹ are selected from the group consisting of hydrogen and C₁-C₈alkyl.

18. The method according to claim 17, wherein the substituted-alkyl compound is selected from:
2,2,9,9-tetramethyldecanedioic acid; and
2,2,12,12-tetramethyltridecanedioic acid;
or a pharmaceutically acceptable salt thereof.

19. A method of treating a disease or disorder selected from arthritis, cartilage damage, joint pain, joint inflammation, systemic lupus erythematous, mixed connective tissue disease, and sepsis in a mammal, comprising administering to the mammal a therapeutically effective amount of a compound selected from:
1,13-Dihydroxy-2,2,12,12-tetramethyl-tridecan-7-one;
2,2,12,12-Tetramethyl-7-oxo-tridecanedioic acid diethyl ester;
1,11-Dihydroxy-2,2,10,10-tetramethyl-undecan-6-one;
2,12-Dimethyl-7-oxo-2,12-di-p-tolyl-tridecanedioic acid;
1,13-Dihydroxy-2,12-dimethyl-2,12-di-p-tolyl-tridecan-7-one;
2,12-Bis-(4-isobutyl-phenyl)-2,12-dimethyl-7-oxo-tridecanedioic acid;
1,13-Dihydroxy-2,12-bis-(4-isobutyl-phenyl)-2,12-dimethyl-tridecan-7-one;
2,10-Dimethyl-6-oxo-2,10-diphenyl-undecanedioic acid;
1,11-Dihydroxy-2,10-dimethyl-2,10-diphenyl-undecan-6-one;
9-Hydroxy-3-(6-hydroxy-5,5-dimethyl-hexyl)-8,8-dimethylnonan-2-one;
Bis[3-(3-hydroxy-2,2-dimethylpropyl)phenyl]methanone;
3-{3-[3-(2-Carboxy-2-methyl-propyl)-benzoyl]-phenyl}-2,2-dimethyl-propanoic acid;
2,2,12,12-Tetramethyl-7-oxo-tridecanedioic acid bis-methylamide;
2,2,12,12-Tetramethyl-7-oxo-tridecanedioic acid bis-phenylamide; and
7-Oxo-2,12-dimethyl-2,12-diphenyl-tridecanedioic acid; or
a pharmaceutically acceptable salt thereof.

20. A method of treating a disease or disorder selected from arthritis, cartilage damage, joint pain, joint inflammation, systemic lupus erythematous, mixed connective tissue disease, and sepsis in a mammal, comprising administering to the mammal a therapeutically effective amount of a compound selected from:
5-[2-(4-Carboxy-4-methyl-pentylsulfanyl)-ethylsulfanyl]-2,2-dimethyl-pentanoic acid;
Bis-(5,5-dimethyl-6-tetrahydropyranyloxy-hexyl)-sulfide;
6-(5,5-Dimethyl-6-hydroxy-hexyl-sulfanyl)-2,2-dimethyl-hexan-1-ol;
Ethyl 5-mercapto-2,2-dimethylpentanoate;
2,2,12,12-Tetramethyl-6,8-dithiatridecane-1,13-dioic acid;
6-(6-Hydroxy-5-methyl-5-phenylhexylsulfanyl)-2-methyl-2-phenylhexan-1-ol;
6-(5-Carboxy-5-phenylhexylsulfanyl)-2-methyl-2-phenyl-hexanoic acid;
Di-(6-hydroxy-5,5-dimethylpentyl)sulfide;
5-(5-Hydroxy-4-methyl-4-phenylpentylsulfanyl)-2-methyl-2-phenylpentan-1-ol; and
2,2,12,12-Tetramethyl-5,9-dithiatridecanedioic acids disodium salt; or
a pharmaceutically acceptable salt thereof.

21. A method of treating a disease or disorder selected from arthritis, cartilage damage, joint pain, joint inflammation, systemic lupus erythematous, mixed connective tissue disease, and sepsis in a mammal, comprising administering to the mammal a therapeutically effective amount of a compound selected from:
6-(5,5-Dimethyl-6-hydroxy-hexane-1-sulfinyl)-2,2-dimethyl-hexan-1-ol;
6-(6-Hydroxy-5-methyl-5-phenylhexylsulfinyl)-2-methyl-2-phenylhexan-1-ol;
5-(5-Hydroxy-4,4-dimethyl-pentyl-1-sulfinyl)-2,2-dimethyl-pentan-1-ol; and
5-(5-Hydroxy-4-methyl-4-phenylpentylsulfinyl)-2-methyl-2-phenylpentan-1-ol; or
a pharmaceutically acceptable salt thereof.

22. A combination, comprising a COX-2 inhibitor selected from celecoxib and valdecoxib, or a pharmaceutically acceptable salt thereof, and a substituted dialkyl ether compound selected from compound named 6-(5-carboxy-5-methyl-hexyloxy)-2,2-dimethyl-hexanoic acid, or a pharmaceutically acceptable salt thereof.

23. The combination according to claim 22, wherein the substituted dialkyl ether is selected from:
6-(5-carboxy-5-methyl-hexyloxy)-2,2-dimethyl-hexanoic acid calcium salt;
6-(5-carboxy-5-methyl-hexyloxy)-2,2-dimethyl-hexanoic acid, calcium salt hydrate;
Crystal Form 1 of 6-(5-carboxy-5-methyl-hexyloxy)-2,2-dimethyl-hexanoic acid, calcium salt; and
Crystal Form 2 of 6-(5-carboxy-5-methyl-hexyloxy)-2,2-dimethyl-hexanoic acid, calcium salt.

24. A pharmaceutical composition comprising
a.) a substituted dialkyl ether compound of Formula I 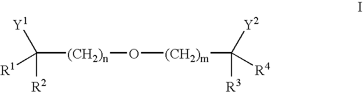
or a pharmaceutically acceptable salt thereof,
wherein:
n and m independently are integers of from 2 to 9;
R¹, R², R³, and R⁴ independently are C₁-C₆ alkyl, C₂-C₆ alkenyl, or C₂-C₆alkynyl; or
R¹ and R² together with the carbon atom to which they are attached, or R³ and R⁴ together with the carbon atom to which they are attached, or R¹ and R² together with the carbon atom to which they are attached or R³ and R⁴ together with the carbon atom to which they are attached, can complete a carbocyclic ring having from 3 to 6 carbons;
Y¹ and Y² independently are COOH, CHO, tetrazole, or COOR⁵, wherein R⁵ is C₁-C₆ alkyl, C₂-C₆ alkenyl, or C₂-C₆ alkynyl; and
wherein the alkyl, alkenyl, and alkynyl groups may be substituted with one or two groups selected from halo, hydroxy, C₁-C₆ alkoxy, and phenyl;
b.) a selective COX-2 inhibitor or a pharmaceutically acceptable salt thereof; and
c.) a pharmaceutically acceptable carrier or diluent.

25. The pharmaceutical composition of claim 24, wherein said dialkyl ether is a compound of Formula I 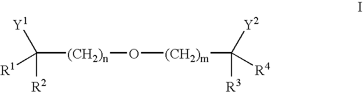
or a pharmaceutically acceptable salt thereof,
wherein:
n and m independently are integers of from 2 to 9;
R¹, R², R³, and R⁴ independently are C₁-C₆ alkyl; and
Y¹ and Y² independently are COOH or COOR⁵, wherein R⁵ is C₁-C₆ alkyl.

26. The pharmaceutical composition of claim 24, wherein said COX-2 inhibitor is LAS-34475;
UR-8880;
ABT-963;
valdecoxib;
BMS-347070;
celecoxib;
tilacoxib;
the compound of formula (B) 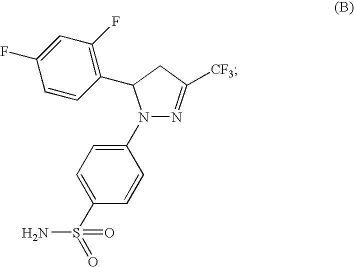
CS-502;
(6aR, 10aR)-3-(1,1-dimethylheptyl)-6a,7,10,10a-tetrahydro-1-hydroxy-6,6-dimethyl-6H-dibenzo[b,d]pyran-9-carboxylic acid;
CV-247;
2(5H)-Furanone, 5,5-dimethyl-3-(1-methylethoxy)-4-[4-(methylsulfonyl)phenyl]-(“DFP”);
carprofen;
deracoxib;
etoricoxib;
GW-406381;
tiracoxib;
meloxicam;
nimesulide;
2-(Acetyloxy)benzoic acid, 3-[(nitrooxy)methyl]phenyl ester;
lumiracoxib;
parecoxib;
P54;
rofecoxib;
revlMiD;
2,6-Bis(1,1-dimethylethyl)-4-[(E)-(2-ethyl-1,1-dioxo-5-isothiazolidinylidene)methyl]phenol;
5(R)-Thio-6-sulfonamide-3(2H)-benzofuranone;
N-[3-(Formylamino)-4-oxo-6-phenoxy-4H-1-benzopyran-7-yl]-methanesulfonamide; or
a pharmaceutically acceptable salt thereof.

27. The pharmaceutical composition of claim 24, wherein said COX-2 inhibitor is carprofen, deracoxib, etoricoxib, lumiracoxib, parecoxib, rofecoxib, valdecoxib, celecoxib, or a pharmaceutically acceptable salt thereof.

28. The pharmaceutical composition of claim 24, wherein said dialkyl ether is a compound of Formula I 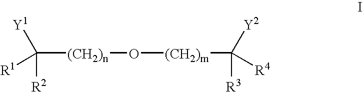
or a pharmaceutically acceptable salt thereof,
wherein:
n and m independently are integers of from 2 to 9;
R¹, R², R³, and R⁴ independently are C₁-C₆ alkyl; and
Y¹ and Y² independently are COOH or COOR⁵, wherein R⁵ is C₁-C₆ alkyl.

29. The pharmaceutical composition of claim 24, wherein said dialkyl ether is 6-(5-carboxy-5-methyl-hexyloxy)-2,2-dimethyl-hexanoic acid, or a pharmaceutically acceptable salt thereof.

30. The pharmaceutical composition of claim 24, wherein said dialkyl ether is 6-(5-carboxy-5-methyl-hexyloxy)-2,2-dimethyl-hexanoic acid, calcium salt.

31. The pharmaceutical composition of claim 24, wherein said dialkyl ether is
6-(5-carboxy-5-methyl-hexyloxy)-2,2-dimethyl-hexanoic acid, calcium salt hydrate;
Crystal Form 1 of 6-(5-carboxy-5-methyl-hexyloxy)-2,2-dimethyl-hexanoic acid, calcium salt; or
Crystal Form 2 of 6-(5-carboxy-5-methyl-hexyloxy)-2,2-dimethyl-hexanoic acid, calcium salt.

32. The pharmaceutical composition of claim 24, wherein said dialkyl ether is 6-(5-carboxy-5-methyl-hexyloxy)-2,2-dimethyl-hexanoic acid, calcium salt and said COX-2 inhibitor is parecoxib, rofecoxib, valdecoxib, or celecoxib.

33. A pharmaceutical composition comprising
a.) a substituted-alkyl compound of Formula II 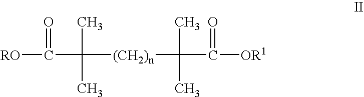
or a pharmaceutically acceptable salt thereof,
wherein n is 6, 7, 8, 9, or 10; and
R and R¹ are selected from the group consisting of hydrogen and C₁-C₈alkyl;
b.) a selective COX-2 inhibitor or a pharmaceutically acceptable salt thereof; and
c.) a pharmaceutically acceptable carrier or diluent.

34. The pharmaceutical composition of claim 33, wherein the substituted-alkyl compound is selected from:
2,2,9,9-tetramethyldecanedioic acid; and
2,2,12,12-tetramethyltridecanedioic acid;
or a pharmaceutically acceptable salt thereof.
